(19) United States
(12) Patent Application Publication
(10) Pub. No.: US 2009/0203725 A1
(43) Pub. Date: Aug. 13, 2009

(54) Androgen Receptor Modulator Compounds and Methods

(76) Inventors: Cornelis Arjan Van Oeveren, San Diego, CA (US); Yixing Shen, San Diego, CA (US); Shuo Zhao, San Diego, CA (US); Lin Zhi, San Diego, CA (US)

Correspondence Address: K&L Gates LLP, 3580 Carmel Mountain Road, Suite 200, San Diego, CA, 92130, US

(21) Appl. No.: 12/084,797

(22) PCT Filed: Dec. 21, 2006
(86) PCT No.: PCT/US2006/048784

§ 371 Date: Feb. 20, 2009

Related U.S. Application Data
(60) Provisional application No. 60/753,302, filed on Dec. 21, 2005.

(51) Int. Cl. A61K 31/4741; C12N 5/02; A61K 31/352; C07D 491/052; C07D 311/78

(52) U.S. Cl. 514/285; 435/375; 514/455; 546/62; 549/280

(57) ABSTRACT

Provided herein are compounds that bind to androgen receptors and/or modulate activity of androgen receptors and/or modulate the amount of androgen receptors; and to methods for making and using such compounds. Also provided are compositions containing such compounds and methods for making and using such compositions.

### RELATED APPLICATIONS

Benefit of priority to the following U.S. Provisional Patent Application is claimed herein: U.S. provisional application 60/753,302 to Van Oeveren et al., filed Dec. 21, 2005, entitled “ANDROGEN RECEPTOR MODULATOR COMPOUNDS AND METHODS.”

For U.S. national stage purposes and where appropriate, the above-noted provisional application is incorporated by reference herein in its entirety.

### FIELD

Provided herein are compounds that bind to androgen receptors and/or modulate activity of androgen receptors and/or modulate the amount of androgen receptors; and to methods for making and using such compounds. Also provided are compositions containing such compounds and methods for making and using such compositions.

### BACKGROUND

Certain intracellular receptors (IRs) have been shown to regulate transcription of certain genes. See e.g., R. M. Evans, Science, 240, 889 (1988). Certain of such IRs are steroid receptors, such as androgen receptors, estrogen receptors, mineralocorticoid receptors, and progesterone receptors. Gene regulation by such receptors typically involves binding of an IR by a ligand.

In certain instances, a ligand binds to an IR, forming a receptor/ligand complex. Such a receptor/ligand complex can then translocate to the nucleus of a cell, where it can bind to the DNA of one or more gene regulatory regions. Once bound to the DNA of a particular gene regulatory region, a receptor/ligand complex can modulate the production of the protein encoded by that particular gene. In certain instances, an androgen receptor/ligand complex regulates expression of certain proteins. In certain instances, an androgen receptor/ligand complex can interact directly with the DNA of a particular gene regulatory region. In certain instances, an androgen receptor/ligand complex can interact with other transcription factors, such as activator protein-1 (AP-1) or nuclear factor κB (NFκB). In certain instances, such interactions result in modulation of transcriptional activation.

### SUMMARY

Compounds, compositions and methods for modulating the activity of androgen receptor are provided. Among the compounds provided herein are compounds that are agonists of androgen receptors. Other compounds provided herein are antagonists of androgen receptors.

Compounds provided herein include (hose that have a structure of Formula I or Formula II and pharmaceutically acceptable sails, esters, acids and prodrugs thereof. Formulae I and II are as follows:

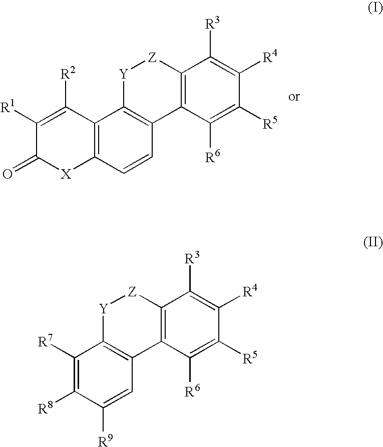

wherein:

R¹ is selected from among hydrogen, F, Cl, C₁-C₄ alkyl, and C₁-C₄ haloalkyl;

R² is selected from among hydrogen, halogen, C₁-C₆ alkyl, C₁-C₆ haloalkyl, C₁-C₆ heteroalkyl, and C₁-C₄ haloheteroalkyl;

R³ and R⁴ are each independently selected from among hydrogen, halogen, C₁-C₆ alkyl, C₁-C₆ haloalkyl, C₁-C₆ heteroalkyl, and C₁-C₆ haloheteroalkyl;

R⁵ and R⁶ are each independently selected from among hydrogen, halogen, OR^{A}, S(O)ₙR^{A}, NR^{A}R^{B}, NR^{A}S(O)ₙR^{B}, COR^{A}, CO₂R^{A}, OC(O)R^{A}, CH₂OR^{A}, CONR^{A}R^{B}, an optionally substituted C₁-C₆ alkyl, an optionally substituted C₁-C₆ haloalkyl, and an optionally substituted C₁-C₆ heteroalkyl; or R⁵ and R⁶ are linked to form a heterocyclic ring;

R⁷ and R⁹ are each independently selected from among hydrogen, halogen, OR^{A}, C₁-C₆ alkyl, C₁-C₆ haloalkyl, C₁-C₆ heteroalkyl, and C₁-C₆ haloheteroalkyl;

R⁸ is selected from among NO₂, CN, COR^{A}, CO₂R^{A}, CONR^{A}R^{B}, SOR^{A}, SO₂R^{A}, SO₂NR^{A}R^{B}, C₁-C₆ haloalkyl, and C₁-C₄ haloheteroalkyl;

R^{A} and R^{B} are each independently selected from among hydrogen, C₁-C₆ alkyl, C₁-C₆ haloalkyl, and C₁-C₆ heteroalkyl; or R^{A} and R^{B} are linked to form a ring;

X is selected from among O, S, and NR^{A};

Y and Z are each independently selected from among CR^{A}R^{B}, CR^{A}OR^{B}, CO, OCH₂, CH₂O, O, S(O)ₙ, and NR^{A}; and

n is selected from among 0, 1, and 2.

In certain embodiments, the compounds provided herein have a structure of Formula I or Formula II:

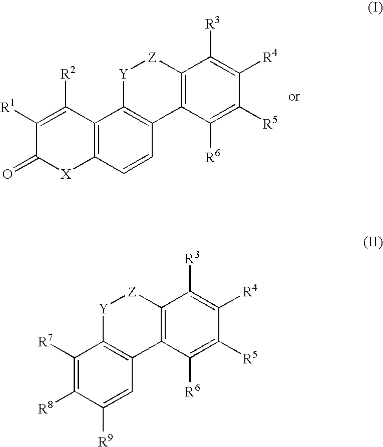

wherein:

R¹ is selected from among hydrogen, F, Cl, C₁-C₄ alkyl, and C₁-C₄ haloalkyl;

R² is selected from among hydrogen, halogen, C₁-C₆ alkyl, C₁-C₆ haloalkyl, C₁-C₆ heteroalkyl, and C₁-C₄ haloheteroalkyl;

R³ and R⁴ are each independently selected from among hydrogen, halogen, C₁-C₆ alkyl, C₁-C₆ haloalkyl, C₁-C₆ heteroalkyl, and C₁-C₆ haloheteroalkyl;

R⁵ and R⁶ are each independently selected from among hydrogen, halogen, OR^{A}, S(O)ₙR^{A}, NR^{A}R^{B}, NR^{A}S(O)ₙR^{B}, COR^{A}, CO₂R^{A}, OC(O)R^{A}, CH₂OR^{A}, CONR^{A}R^{B}, an optionally substituted C₁-C₆ alkyl, an optionally substituted C₁-C₆ haloalkyl, and an optionally substituted C₁-C₆ heteroalkyl; or R⁵ and R⁶ are linked to form a heterocyclic ring;

R⁷ and R⁹ are each independently selected from among hydrogen, halogen, OR^{A}, C₁-C₆ alkyl, C₁-C₆ haloalkyl, C₁-C₆ heteroalkyl, and C₁-C₆ haloheteroalkyl;

R⁸ is selected from among NO₂, CN, COR^{A}, CO₂R^{A}, CONR^{A}R^{B}, SOR^{A}, SO₂R^{A}, SO₂NR^{A}R^{B}, C₁-C₆ haloalkyl, and C₁-C₄ haloheteroalkyl;

R^{A} and R^{B} are each independently selected from among hydrogen, C₁-C₆ alkyl, C₁-C₆ haloalkyl, and C₁-C₆ heteroalkyl; or R^{A} and R^{B} are linked to form a ring;

X is selected from among O, S, and NR^{A};

Y and Z are each independently selected from among CR^{A}R^{B}, CR^{A}OR^{B}, CO, OCH₂, CH₂O, O, S(O)ₙ, and NR^{A};

n is selected from among 0, 1, and 2; and

provided that, if Y is CO, and Z is O, and R⁸ is NO₂, and R⁹ is hydrogen, then R⁷ is not hydrogen; and

provided that, if the compound has a structure of Formula II, then R⁵ and R⁶ are not hydrogen at the same time; and

pharmaceutically acceptable salts and prodrugs thereof.

In certain embodiments, if X is NH, and Y is CO, and Z is O, and each of R¹, R³, R⁴, and R⁵ is hydrogen, and R² is CH₃, then R⁶ is not OCH₃.

In certain embodiments, the compounds provided herein have a structure of Formula (I):

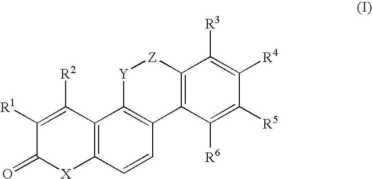

wherein:

R¹ is selected from among hydrogen, F, Cl, C₁-C₄ alkyl, and C₁-C₄ haloalkyl;

R² is selected from among hydrogen, halogen, C₁-C₆ alkyl, C₁-C₆ haloalkyl, C₁-C₆ heteroalkyl, and C₁-C₄ haloheteroalkyl;

R³ and R⁴ are each independently selected from among hydrogen, halogen, C₁-C₆ alkyl, C₁-C₆ haloalkyl, C₁-C₆ heteroalkyl, and C₁-C₆ haloheteroalkyl;

R⁵ and R⁶ are each independently selected from among hydrogen, halogen, OR^{A}, S(O)ₙR^{A}, NR^{A}R^{B}, NR^{A}S(O)ₙR^{B}, COR^{A}, CO₂R^{A}, OC(O)R^{A}, CH₂OR^{A}, CONR^{A}R^{B} an optionally substituted C₁-C₆ alkyl, an optionally substituted C₁-C₆ haloalkyl, and an optionally substituted C₁-C₆ heteroalkyl; or R⁵ and R⁶ are linked to form a heterocyclic ring;

R^{A} and R^{B} are each independently selected from among hydrogen, C₁-C₆ alkyl, C₁-C₆ haloalkyl, and C₁-C₆ heteroalkyl; or R^{A} and R^{B} are linked to form a ring;

X is selected from among O, S, and NR^{A};

Y and Z are each independently selected from among CR^{A}R^{B}, CR^{A}OR^{B}, CO, OCH₂, CH₂O, O, S(O)ₙ, and NR^{A};

n is selected from among 0, 1, and 2; and

pharmaceutically acceptable salts and prodrugs thereof.

In certain embodiments, if X is NH, and Y is CO, and Z is O, and each of R¹, R³, R⁴, and R⁵ is hydrogen, and R² is CH₃, then R⁶ is not OCH₃.

In certain embodiments, the compounds provided herein have a structure of Formula II;

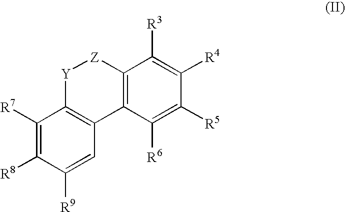

wherein:

R³ and R⁴ are each independently selected from among hydrogen, halogen, C₁-C₆ alkyl, C₁-C₆ haloalkyl, C₁-C₆ heteroalkyl, and C₁-C₆ haloheteroalkyl;

R⁵ and R⁶ are each independently selected from among hydrogen, halogen, OR^{A}, S(O)ₙR^{A}, NR^{A}R^{B}, NR^{A}S(O)ₙR^{B}, COR^{A}, CO₂R^{A}, OC(O)R^{A}, CH₂OR^{A}, CONR^{A}R^{B}, an optionally substituted C₁-C₆ alkyl, an optionally substituted C₁-C₆ haloalkyl, and an optionally substituted C₁-C₆ heteroalkyl; or R⁵ and R⁶ are linked to form a heterocyclic ring;

R⁷ and R⁹ are each independently selected from among hydrogen, halogen, OR^{A}, C₁-C₆ alkyl, C₁-C₆ haloalkyl, C₁-C₆ heteroalkyl, and C₁-C₆ haloheteroalkyl;

R⁸ is selected from among NO₂, CN, COR^{A}, CO₂R^{A}, CONR^{A}R^{B}, SOR^{A}, SO₂R^{A}, SO₂NR^{A}R^{B}, C₁-C₆ haloalkyl, and C₁-C₄ haloheteroalkyl;

R^{A} and R^{B} are each independently selected from among hydrogen, C₁-C₆ alkyl, C₁-C₆ haloalkyl, and C₁-C₆ heteroalkyl; or R^{A} and R^{B} are linked to form a ring;

Y and Z are each independently selected from among CR^{A}R^{B}, CR^{A}OR^{B}, CO, OCH₂, CH₂O, O, S(O)ₙ, and NR^{A};

n is selected from among 0, 1, and 2; and

provided that, if Y is CO, and Z is O, and R⁸ is NO₂, and R⁹ is hydrogen, then R⁷ is not hydrogen; and

provided that R⁵ and R⁶ are not hydrogen at the same time; and

pharmaceutically acceptable salts and prodrugs thereof.

In certain embodiments, the compounds provided herein have a structure of Formula I or Formula II:

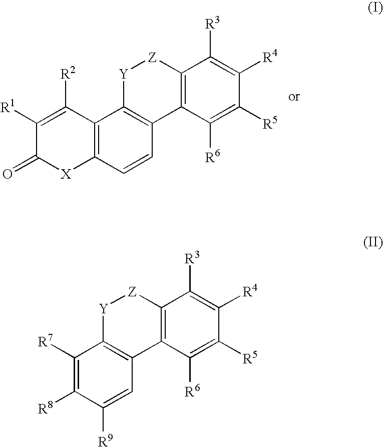

wherein:

R¹ is selected from among hydrogen, F, Cl, C₁-C₄ alkyl, and C₁-C₄ haloalkyl;

R² is selected from among hydrogen, halogen, C₁-C₆ alkyl, C₁-C₆ haloalkyl, C₁-C₆ heteroalkyl, and C₁-C₄ haloheteroalkyl;

R³ and R⁴ are each independently selected from among hydrogen, halogen, C₁-C₆ alkyl, C₁-C₆ haloalkyl, C₁-C₆ heteroalkyl, and C₁-C₆ heteroalkyl;

R⁵ and R⁶ are each independently selected from among hydrogen, halogen, OR^{A}, S(O)ₙR^{A}, NR^{A}R^{B}, NR^{A}S(O)ₙR^{B}, COR^{A}, CO₂R^{A}, OC(O)R^{A}, CH₂OR^{A}, CONR^{A}R^{B}, an optionally substituted C₁-C₆ alkyl, an optionally substituted C₁-C₆ haloalkyl, and an optionally substituted C₁-C₆ heteroalkyl; or R⁵ and R⁶ are linked to form a heterocyclic ring;

R⁷ and R⁹ are each independently selected from among hydrogen, halogen, OR^{A}, C₁-C₆ alkyl, C₁-C₆ haloalkyl, C₁-C₆ heteroalkyl, and C₁-C₆ haloheteroalkyl;

R⁸ is selected from among NO₂, CN, COR^{A}, CO₂R^{A}, CONR^{A}R^{B}, SOR^{A}, SO₂R^{A}, SO₂NR^{A}R^{B}, C₁-C₆ haloalkyl, and C₁-C₄ haloheteroalkyl;

R^{A} and R^{B} are each independently selected from among hydrogen, C₁-C₆ alkyl, C₁-C₆ haloalkyl, and C₁-C₆ heteroalkyl; or R^{A} and R^{B} are linked to form a ring;

X is selected from among O, S, and NR^{A};

Y and Z are each independently selected from among CR^{A}R^{B}, CR^{A}OR^{B}, CO., OCH₂, CH₂O, O, S(O)ₙ and NR^{A};

n is selected from among 0, 1, and 2; and

provided that, if R⁸ is NO₂, CN, COR^{A}, CO₂R^{A} or CONR^{A}R^{B}, and Y or Z is CO, and R⁹ is hydrogen, then R⁷ is not hydrogen; and

provided that, if R⁸ is COR^{A} or CO₂R^{A}, and R⁹ is methoxy, then R⁷ is not hydrogen; and

provided that, if R⁸ is CN or CO₂R^{A}, and R⁷ is methyl, then one of R⁵, R⁶ or R⁹ is not hydrogen; and

provided that, if the compound has a structure of Formula II, then Y and Z are not the same; and

pharmaceutically acceptable salts and prodrugs thereof.

In certain embodiments, if X is NH, and Y is CO, and Z is O, and each of R¹, R³, R⁴, and R⁵ is hydrogen, and R² is CH₃, then R⁶ is not OCH₃.

In certain embodiments, if R⁸ is CN or COR^{A}, and R⁷ is methyl, then R⁹ is not hydrogen.

In certain embodiments, the compounds provided herein have a structure of Formula II:

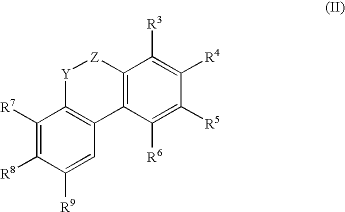

wherein:

R³ and R⁴ are each independently selected from among hydrogen, halogen, C₁-C₆ alkyl, C₁-C₆ haloalkyl, C₁-C₆ heteroalkyl, and C₁-C₆ haloheteroalkyl;

R⁵ and R⁶ are each independently selected from among hydrogen, halogen, OR^{A}, S(O)ₙR^{A}, NR^{A}R^{B}, NR^{A}S(O)ₙR^{B}, COR^{A}, CO₂R^{A}, OC(O)R^{A}, CH₂OR^{A}, CONR^{A}R^{B}, an optionally substituted C₁-C₆ alkyl, an optionally substituted C₁-C₆ haloalkyl, and an optionally substituted C₁-C₆ heteroalkyl; or R⁵ and R⁶ are linked to form a heterocyclic ring;

R⁷ and R⁹ are each independently selected from among hydrogen, halogen, OR^{A}, C₁-C₆ alkyl, C₁-C₆ haloalkyl, C₁-C₆ heteroalkyl, and C₁-C₆ haloheteroalkyl;

R⁸ is selected from among NO₂, CN, COR^{A}, CO₂R^{A}, CONR^{A}R^{B}, SOR^{A}, SO₂R^{A}, SO₂NR^{A}R^{B}, C₁-C₆ haloalkyl, and C₁-C₄ haloheteroalkyl;

R^{A} and R^{B} are each independently selected from among hydrogen, C₁-C₆ alkyl, C₁-C₆ haloalkyl, and C₁-C₆ heteroalkyl; or R^{A} and R^{B} are linked to form a ring;

Y and Z are each independently selected from among CR^{A}R^{B}, CR^{A}OR^{B}, CO, OCH₂, CH₂O, O, S(O)ₙ, and NR^{A};

n is selected from among 0, 1, and 2;

provided that, if R⁸ is NO₂, CN, COR^{A}, CO₂R^{A} or CONR^{A}R^{B}, and R⁹ is hydrogen, then R⁷ is not hydrogen;

provided that, if R⁸ is COR^{A} or CO₂R^{A}, and R⁹ is methoxy, then R⁷ is not hydrogen;

provided that, if R⁸ is CN or CO₂R^{A}, and R⁷ is methyl, then one of R⁵, R⁶ or R⁹ is not hydrogen; and

provided that, if the compound has a structure of Formula II, then Y and Z are not the same; and

pharmaceutically acceptable salts and prodrugs thereof.

In certain embodiments, the compounds provided herein have a structure of Formula II:

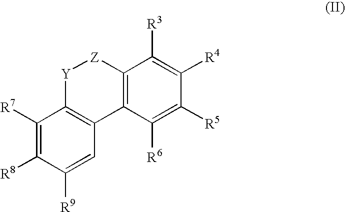

wherein:

R³ and R⁴ are each independently selected from among hydrogen, halogen, C₁-C₆ alkyl, C₁-C₆ haloalkyl, C₁-C₆ heteroalkyl, and C₁-C₆ haloheteroalkyl;

R⁵ and R⁶ are each independently selected from among hydrogen, halogen, OR^{A}, S(O)ₙR^{A}, NR^{A}R^{B}, NR^{A}S(O)ₙR^{B}, COR^{A}, CO₂R^{A}, OC(O)R^{A}, CH₂OR^{A}, CONR^{A}R^{B}, an optionally substituted C₁-C₆ alkyl, an optionally substituted C₁-C₆ haloalkyl, and an optionally substituted C₁-C₆ heteroalkyl; or R⁵ and R⁶ are linked to form a heterocyclic ring;

R⁷ and R⁹ are each independently selected from among hydrogen, halogen, OR^{A}, C₁-C₆ alkyl, C₁-C₆ haloalkyl, C₁-C₆ heteroalkyl, and C₁-C₆ haloheteroalkyl;

R⁸ is selected from among NO₂, CN, COR^{A}, CO₂R^{A}, CONR^{A}R^{B}, SOR^{A}, SO₂R^{A}, SO₂NR^{A}R^{B}, C₁-C₆ haloalkyl, and C₁-C₄ haloheteroalkyl;

R^{A} and R^{B} are each independently selected from among hydrogen, C₁-C₆ alkyl, C₁-C₆ haloalkyl, and C₁-C₆ heteroalkyl; or R^{A} and R^{B} are linked to form a ring;

Y and Z are each independently selected from among CR^{A}R^{B}, CR^{A}OR^{B}, CO, OCH₂, CH₂O, O, S(O)ₙ, and NR^{A};

n is selected from among 0, 1, and 2;

provided that, if R⁸ is NO₂, CN, COR^{A}, CO₂R^{A} or CONR^{A}R^{B}, and R⁹ is hydrogen, then R⁷ is not hydrogen;

provided that, if R⁸ is COR^{A} or CO₂R^{A}, and R⁹ is methoxy, then R⁷ is not hydrogen;

provided that, if R⁸ is CN or CO₂R^{A}, and R⁷ is methyl, then one of R⁶ or R⁹ is not hydrogen; and

provided that, if the compound has a structure of Formula II, then Y and Z are not the same; and

pharmaceutically acceptable salts and prodrugs thereof.

In certain embodiments, the compounds provided herein have a structure of Formula II:

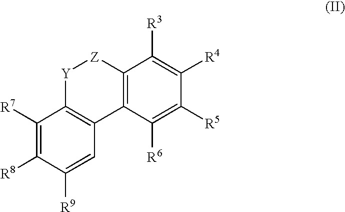

wherein;

R³ and R⁴ are each independently selected from among hydrogen, halogen, C₁-C₆ alkyl, C₁-C₆ haloalkyl, C₁-C₆ heteroalkyl, and C₁-C₆ haloheteroalkyl;

R⁵ and R⁶ are each independently selected from among hydrogen, halogen, OR^{A}, S(O)ₙR^{A}, NR^{A}R^{B}, NR^{A}S(O)ₙR^{B}, COR^{A}, CO₂R^{A}, OC(O)R^{A}, CH₃₀R^{A}, CONR^{A}R^{B}, an optionally substituted C₁-C₆ alkyl, an optionally substituted C₁-C₆ haloalkyl, and an optionally substituted C₁-C₆ heteroalkyl; or R⁵ and R⁶ are linked to form a heterocyclic ring;

R⁷ and R⁹ are each independently selected from among hydrogen, halogen, OR^{A}, C₁-C₆ alkyl, C₁-C₆ haloalkyl, C₁-C₆ heteroalkyl, and C₁-C₆ haloheteroalkyl;

R⁸ is selected from among NO₂, CN, COR^{A}, CO₂R^{A}, CONR^{A}R^{B}, SOR^{A}, SO₂R^{A}, SO₂NR^{A}R^{B}, C₁-C₆ haloalkyl, and C₁-C₄ haloheteroalkyl;

R^{A} and R^{B} are each independently selected from among hydrogen, C₁-C₆ alkyl, C₁-C₆ haloalkyl, and C₁-C₆ heteroalkyl; or R^{A} and R^{B} are linked to form a ring;

Y and Z are each independently selected from among CR^{A}R^{B}, CR^{A}OR^{B}, CO, OCH₂, CH₂O, O, S(O)ₙ, and NR^{A};

n is selected from among 0, 1, and 2;

provided that, if R⁸ is NO₂, CN, COR^{A}, CO₂R^{A} or CONR^{A}R^{B}, and R⁹ is hydrogen, then R⁷ is not hydrogen;

provided that, if R⁸ is COR^{A} or CO₂R^{A}, and R⁹ is methoxy, then R⁷ is not hydrogen;

provided that, if R⁸ is CN or CO₂R^{A}, and R⁷ is methyl, then R⁹ is not hydrogen; and

provided that, if the compound has a structure of Formula II, then Y and Z are not the same; and

pharmaceutically acceptable salts and prodrugs thereof.

In certain embodiments, the compounds provided herein have a structure of Formula III;

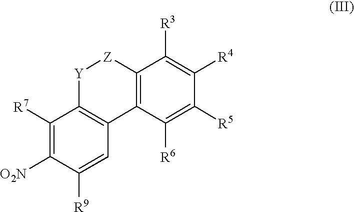

wherein:

R³ and R⁴ are each independently selected from among hydrogen, halogen, C₁-C₆ alkyl, C₁-C₆ haloalkyl, C₁-C₆ heteroalkyl, and C₁-C₆ haloheteroalkyl;

R⁵ and R⁶ are each independently selected from among hydrogen, halogen, OR^{A}, S(O)ₙR^{A}, NR^{A}R^{B}, NR^{A}S(O)ₙR^{B}, COR^{A}, CO₂R^{A}, OC(O)R^{A}, CH₂OR^{A}, CONR^{A}R^{B}, an optionally substituted C₁-C₆ alkyl, an optionally substituted C₁-C₆ haloalkyl, and an optionally substituted C₁-C₆ heteroalkyl; or R⁵ and R⁶ are linked to form a heterocyclic ring;

R⁷ and R⁹ are each independently selected from among hydrogen, halogen, OR^{A}, C₁-C₆ alkyl, C₁-C₆ haloalkyl, C₁-C₆ heteroalkyl, and C₁-C₆ haloheteroalkyl;

R^{A} and R^{B} are each independently selected from among hydrogen, C₁-C₆ alkyl, C₁-C₆ haloalkyl, and C₁-C₆ heteroalkyl; or R^{A} and R^{B} are linked to form a ring;

Y and Z are each independently selected from among CR^{A}R^{B}, CR^{A}OR^{B}, CO, OCH₂, CH₂O, O, S(O)ₙ, and NR^{A};

n is selected from among 0, 1, and 2;

provided that, if R⁹ is hydrogen, then R⁷ is not hydrogen; and

provided that Y and Z are not the same; and

pharmaceutically acceptable salts and prodrugs thereof.

In certain embodiments, the compounds provided herein have a structure of Formula III:

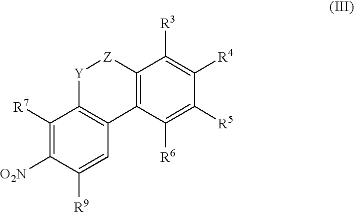

wherein:

R³ and R⁴ are each independently selected from among hydrogen, halogen, C₁-C₆ alkyl, C₁-C₆ haloalkyl, C₁-C₆ heteroalkyl, and C₁-C₆ haloheteroalkyl;

R⁵ and R⁶ are each independently selected from among hydrogen, halogen, OR^{A}, S(O)ₙR^{A}, NR^{A}R^{B}, NR^{A}S(O)ₙR^{B}, COR^{A}, CO₂R^{A}, OC(O)R^{A}, CH₂OR^{A}, CONR^{A}R^{B}, an optionally substituted C₁-C₆ alkyl, an optionally substituted C₁-C₆ haloalkyl, and an optionally substituted C₁-C₆ heteroalkyl; or R⁵ and R⁶ are linked to form a heterocyclic ring;

R⁷ and R⁹ are each independently selected from among hydrogen, halogen, OR^{A}, C₁-C₆ alkyl, C₁-C₆ haloalkyl, C₁-C₆ heteroalkyl, and C₁-C₆ haloheteroalkyl;

R^{A} and R^{B} are each independently selected from among hydrogen, C₁-C₆ alkyl, C₁-C₆ haloalkyl, and C₁-C₆ heteroalkyl; or R^{A} and R^{B} are linked to form a ring;

Y and Z are each independently selected from among CR^{A}R^{B}, CR^{A}OR^{B}, CO, OCH₂, CH₂O, O, S(O)ₙ, and NR^{A};

is selected from among 0, 1, and 2;

provided that, if Y is CO and Z is O and R⁷ is hydrogen, then R⁹ is not hydrogen;

provided that, if Y is O and Z is CO and R⁷ is hydrogen, then one of R³, R⁴, R⁵, R⁶ or R⁹ is not hydrogen; and

provided that Y and Z are not the same; and

pharmaceutically acceptable salts and prodrugs thereof.

For any and all of the embodiments, substituents can be selected from among from n subset of the listed alternatives. For example, in some embodiments, R¹ is selected from among hydrogen, F, Cl, C₁-C₄ alkyl, and C₁-C₄ haloalkyl. In other embodiments, R¹ is selected from among hydrogen, F, Cl, an optionally substituted C₁-C₄ alkyl, and an optionally substituted C₁-C₄ haloalkyl. In certain embodiments, R¹ is selected from among hydrogen, F, Cl, a fully saturated C₁-C₄ alkyl, and a fully saturated C₁-C₄ haloalkyl, R¹ can be selected from among hydrogen, F, Cl and C₁-C₄ alkyl. In certain embodiments, R¹ is selected from among hydrogen, F, and Cl In certain embodiments, R¹ is selected from among hydrogen and C₁-C₄ alkyl. In certain embodiments, R¹ is hydrogen.

In certain embodiments, R² is selected from among hydrogen, halogen, C₁-C₆ alkyl, C₁-C₆ haloalkyl, C₁-C₆ heteroalkyl, and C₁-C₄ haloheteroalkyl. In certain embodiments, R² is selected from among hydrogen, halogen, an optionally substituted C₁-C₆ alkyl, an optionally substituted C₁-C₆ haloalkyl, an optionally substituted C₁-C₆ heteroalkyl, and an optionally substituted C₁-C₄ haloheteroalkyl.

In certain embodiments, R² is selected from among hydrogen, halogen, C₁-C₆ alkyl and C₁-C₆ haloalkyl. In certain embodiments, R² is selected from among halogen, C₁-C₆ alkyl, C₁-C₆ haloalkyl C₁-C₆ heteroalkyl, and C₁-C₄ haloheteroalkyl. In certain embodiments, R² is selected from among halogen, C₁-C₆ alkyl and C₁-C₆ haloalkyl. In certain embodiments, R² is selected from among C₁-C₆ alkyl and C₁-C₆ haloalkyl. In certain embodiments, R² is selected from among methyl and trifluoromethyl. In certain embodiments, R² is methyl. In certain embodiments, R² is trifluoromethyl.

In certain embodiments, R³ and R⁴ are each independently selected from among hydrogen, halogen, C₁-C₆ alkyl, C₁-C₆ haloalkyl, C₁-C₆ heteroalkyl, and C₁-C₆ haloheteroalkyl. In certain embodiments, R³ and R⁴ are each independently selected from among hydrogen, halogen, an optionally substituted C₁-C₆ alkyl, an optionally substituted C₁-C₆ haloalkyl, an optionally substituted C₁-C₆ heteroalkyl, and an optionally substituted C₁-C₆ haloheteroalkyl.

In certain embodiments, R³ and R⁴ are each independently selected from among hydrogen, halogen, C₁-C₆ alkyl and C₁-C₆ haloalkyl. In certain embodiments, R³ and R⁴ are each independently selected from among hydrogen, halogen and C₁-C₆ alkyl. In certain embodiments, R³ and R⁴ are each independently hydrogen.

In certain embodiments, R⁵ and R⁶ are each independently selected from among hydrogen, halogen, OR^{A}, S(O)ₙR^{A}, NR^{A}R^{B}, NR^{A}S(O)ₙR^{B}, COR^{A}, CO₂R^{A}, OC(O)R^{A}, CH₂OR^{A}, CONR^{A}R^{B}, an optionally substituted C₁-C₆ alkyl, an optionally substituted C₁-C₆ haloalkyl, and an optionally substituted C₁-C₆ heteroalkyl. In certain embodiments, R⁵ and R⁶ are linked to form a heterocyclic ring.

In certain embodiments, R⁵ and R⁶ are each independently selected from among hydrogen, halogen, OR^{A}, COR^{A}, CO₂R^{A}, OC(O)R^{A}, CH₂OR^{A}, C₁-C₆ alkyl, C₁-C₆ haloalkyl, and C₁-C₆ heteroalkyl. In certain embodiments, R⁵ and R⁶ are each independently selected from among hydrogen, halogen, OR^{A}, COR^{A}, CO₂R^{A}, OC(O)R^{A}, and CH₂OR^{A}. In certain embodiments, R⁵ and R⁶ are each independently selected from among hydrogen, halogen, OR^{A}, OC(O)R^{A}, and CH₂OR^{A}. In certain embodiments, R⁵ and R⁶ are each independently selected from among hydrogen, OR^{A} and OC(O)R^{A}.

In certain embodiments, R⁵ and R⁶ are each independently selected from among hydrogen, F, hydroxy, methoxy, ethoxy, isopropoxy, hydroxymethyl, OC(O)^{t}Bu and CO₂Me. In certain embodiments, R⁵ and R⁶ are each independently selected from among hydrogen, hydroxy, methoxy and OC(O)^{t}Bu.

In certain embodiments, R⁷ and R⁹ are each independently selected from among hydrogen, halogen, OR^{A}, C₁-C₆ alkyl, C₁-C₆ haloalkyl, C₁-C₆ heteroalkyl, and C₁-C₆ haloheteroalkyl.

In certain embodiments, R⁷ and R⁹ are each independently selected from among hydrogen, halogen and C₁-C₆ alkyl In certain embodiments, R⁷ and R⁹ are each independently selected from among hydrogen and C₁-C₆ alkyl. In certain embodiments, R⁷ and R⁹ are each independently selected from among hydrogen and methyl. In certain embodiments, R⁷ is methyl and R⁹ is hydrogen. In certain embodiments, R⁷ is methyl.

In certain embodiments, R⁸ is selected from among NO₂, CN, COR^{A}, CO₂R^{A}, CONR^{A}R^{B}, SOR^{A}, SO₂R^{A}, SO₂NR^{A}R^{B}, C₁-C₆ haloalkyl, and C₁-C₄ haloheteroalkyl. In certain embodiments, R⁸ is selected from among NO₂, CN, COR^{A}, CO₂R^{A}, CONR^{A}R^{B}, SOR^{A}, SO₂R^{A}, and SO₂NR^{A}R^{B}. In certain embodiments, R⁸ is selected from among NO₂, SOR^{A}, SO₂R^{A}, and SO₂NR^{A}R^{B}. In certain embodiments, R⁸ is selected from among NO₂, C₁-C₆ haloalkyl, and C₁-C₄ haloheteroalkyl. In certain embodiments, R⁸ is NO₂.

In certain embodiments, R^{A} and R^{B} are each independently selected from among hydrogen, C₁-C₆ alkyl, C₁-C₆ haloalkyl, and C₁-C₆ heteroalkyl. In certain embodiments, R^{A} and R^{B} are linked to form a ring. In certain embodiments, R^{A} and R^{B} are each independently selected from among hydrogen and C₁-C₆ alkyl. In certain embodiments, R^{A} and R^{B} are each independently selected from among hydrogen and methyl.

In certain embodiments, X is selected from among O, S, and NR^{A}. In certain embodiments, X is selected from among O and NR^{A}. In certain embodiments, X is NR^{A}.

In certain embodiments, Y and Z are each independently selected from among CR^{A}R^{B}, CR^{A}OR^{B}, CO, OCH₂, CH₂O, O, S(O)ₙ, and NR^{A} in certain embodiments, Y and Z are each independently selected from among CR^{A}R^{B}, CR^{A}OR^{B}, CO, OCH₂, CH₂O and O. In certain embodiments, Y and Z are each independently selected from among CR^{A}R^{B}, CR^{A}OR^{B}, CO and O. In certain embodiments, Y and Z are each independently selected from among CR^{A}OR^{B}, CO and O. In certain embodiments, Y and Z are not the same. In certain embodiments, Y and Z are the same.

In certain embodiments, n is selected from among 0, 1, and 2.

In certain embodiments, if X is NH, and Y is CO, and Z is O, and each of R¹, R³, R⁴, and R⁵ is hydrogen, and R² is CH₃, then R⁶ is not OCH₃.

In certain embodiments, if Y is CO, and Z is O, and R⁸ is NO₂, and R⁹ is hydrogen, then R⁷ is not hydrogen.

In certain embodiments, if R⁸ is NO₂, CN, COR^{A}, CO₂R^{A} or CONR^{A}R^{B}, and R⁹ is hydrogen, then R⁷ is not hydrogen. In certain embodiment, if R⁸ is COR^{A} or CO₂R^{A}, and R⁹ is methoxy, then R⁷ is not hydrogen. In certain embodiments, if R⁸ is CN or COR^{A}, and R⁷ is methyl, then R⁹ is not hydrogen. In certain embodiments, if R⁸ is NO₂, CN, COR^{A}, CO₂R^{A} or CONR^{A}R^{B}, and R⁹ is hydrogen, then R⁷ is not hydrogen; or if R⁸ is COR^{A} or CO₂R^{A}, and R⁹ is methoxy, then R⁷ is not hydrogen; or if R⁸ is CN or COR^{A}, and R⁷ is methyl, then R⁹ is not hydrogen. In certain embodiments, if R⁸ is NO₂, CN, COR^{A}, CO₂R^{A} or CONR^{A}R^{B}, and R⁹ is hydrogen, then R⁷ is not hydrogen; or if R⁸ is COR^{A} or CO₂R^{A}, and R⁹ is methoxy, then R⁷ is not hydrogen; or if R⁸ is CN or COR^{A}, and R⁷ is methyl, then R⁹ is not hydrogen; or if the compound has a structure of Formula II, then Y and Z are not the same. In certain embodiments, if R⁸ is NO₂, CN, COR^{A}, CO₂R^{A}, CONR^{A}R^{B}, SOR^{A}, SO₂R^{A}, SO₂NR^{A}R^{B}, C₁-C₆ haloalkyl, or C₁-C₄ haloheteroalkyl, then Y and Z are not the same.

In certain embodiments, the compounds provided herein have a structure of Formula I or Formula II:

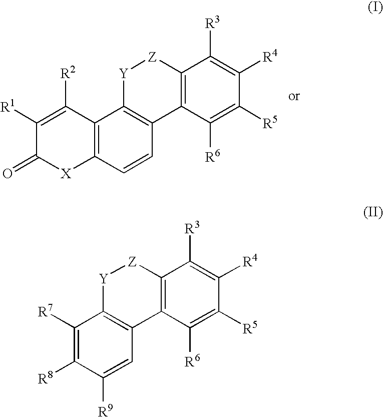

wherein:

R¹ is selected from among hydrogen, F, Cl, C₁-C₄ alkyl, and C₁-C₄ haloalkyl;

R² is selected from among hydrogen, halogen, C₁-C₆ alkyl, C₁-C₆ haloalkyl, C₁-C₆ heteroalkyl, and C₁-C₄ haloheteroalkyl;

R³ and R⁴ are each independently selected from among hydrogen, halogen, C₁-C₆ alkyl, C₁-C₆ haloalkyl, C₁-C₆ heteroalkyl, and C₁-C₆ haloheteroalkyl;

R⁵ and R⁶ are each independently selected from among hydrogen, halogen, OR^{A}, S(O)ₙR^{A}, NR^{A}R^{B}, NR^{A}S(O)ₙR^{B}, COR^{A}, CO₂R^{A}, OC(O)R^{A}, CH₂OR^{A}, CONR^{A}R^{B}, an optionally substituted C₁-C₆ alkyl, an optionally substituted C₁-C₆ haloalkyl, and an optionally substituted C₁-C₆ heteroalkyl; or R⁵ and R⁶ are linked to form a heterocyclic ring;

R⁷ and R⁹ are each independently selected from among hydrogen, halogen, OR^{A}, C₁-C₆ alkyl, C₁-C₆ haloalkyl, C₁-C₆ heteroalkyl, and C₁-C₆ haloheteroalkyl;

R⁸ is selected from among NO₂, CN, COR^{A}, CO₃R^{A}, CONR^{A}R^{B}, SOR^{A}, SO₂R^{A}, and SO₂NR^{A}R^{B}, C₁-C₆ haloalkyl and C₁-C₄ haloheteroalkyl;

R^{A} and R^{B} are each independently selected from among hydrogen, C₁-C₆ alkyl, C₁-C₆ haloalkyl, and C₁-C₆ heteroalkyl; or R^{A} and R^{B} are linked to form a ring;

X is selected from among O, S, and NR^{A};

Y and Z are each independently selected from among CR^{A}R^{B}, CR^{A}OR^{B}, CO, OCH₂, CH₂O, O, S(O)ₙ, and NR^{A};

n is selected from among 0, 1, and 2; and

provided that, if the compound has a structure of Formula II and if Y is the same as Z, then R⁴ is not isopropyl;

provided that, if the compound has a structure of Formula II, then at least one of R³, R⁴, R⁵, R⁶, and R⁹ is not hydrogen; and

provided that, if R⁸ is NO₂ and Y is CO and Z is O and R⁷ is hydrogen, then R⁹ is not hydrogen; and

provided that, if R⁸ is CO₂R^{A} or CONR^{A}R^{B}, then R⁴ is not F or R⁹ is not methoxy; and

pharmaceutically acceptable salts and prodrugs thereof.

In curtain embodiments, if X is NH, and Y is CO, and Z is O, and each of R¹, R³, R⁴, and R⁵ is hydrogen, and R² is CH₃, then R⁶ is not OCH₃.

In certain embodiments, if the compound has a structure of Formula II, and if Y is the same as Z, then R⁴ is not C₁-C₆ alkyl.

In certain embodiments, the compounds provided herein have a structure of Formula II;

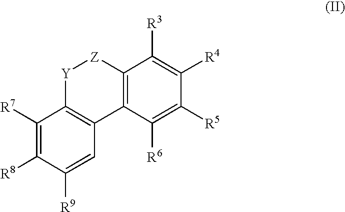

wherein:

R³ and R⁴ are each independently selected from among hydrogen, halogen, C₁-C₆ alkyl, C₁-C₆ haloalkyl, C₁-C₆ heteroalkyl, and C₁-C₆ haloheteroalkyl;

R⁵ and R⁶ are each independently selected from among hydrogen, halogen, OR^{A}, S(O)ₙR^{A}, NR^{A}R^{B}, NR^{A}S(O)ₙR^{B}, COR^{A}, CO₂R^{A}, OC(O)R^{A}, CH₂OR^{A}, CONR^{A}R^{B}, an optionally substituted C₁-C₆ alkyl, an optionally substituted C₁-C₆ haloalkyl, and an optionally substituted C₁-C₆ heteroalkyl; or

R⁵ and R⁶ are linked to form a heterocyclic ring;

R⁷ and R⁹ are each independently selected from among hydrogen, halogen, OR^{A}, C₁-C₆ alkyl, C₁-C₆ haloalkyl, C₁-C₆ heteroalkyl, and C₁-C₆ haloheteroalkyl;

R⁸ is selected from among NO₂, CN, COR^{A}, CO₂R^{A}, CONR^{A}R^{B}, SOR^{A}, SO₂R^{A}, and SO₂NR^{A}R^{B}, C₁-C₆ haloalkyl, and C₁-C₄ haloheteroalkyl;

R^{A} and R^{B} are each independently selected from among hydrogen, C₁-C₆ alkyl, C₁-C₆ haloalkyl, and C₁-C₆ heteroalkyl; or

R^{A} and R^{B} are linked to form a ring;

Y and Z are each independently selected from among CR^{A}R^{B}, CR^{A}OR^{B}, CO, OCH₂, CH₂O, O, S(O)ₙ, and NR^{A};

n is selected from among 0, 1, and 2;

provided that, if Y is the same as Z, then R⁴ is not isopropyl;

provided that at least one of R³, R⁴, R⁵, R⁶, and R⁹ is not hydrogen; and

provided that, if R⁸ is NO₂ and Y is CO and Z is O and R⁷ is hydrogen, then R⁹ is not hydrogen; and

provided that, if R⁸ is CO₂R^{A} or CONR^{A}R^{B}, then R⁴ is not F or R⁹ is not methoxy.

In certain embodiments, R³ and R⁴ are each independently selected from among hydrogen, halogen, C₁-C₆ alkyl, C₁-C₆ haloalkyl, C₁-C₆ heteroalkyl, and C₁-C₆ haloheteroalkyl; R⁵ and R⁶ are each independently selected from among hydrogen, halogen, OR^{A}, COR^{A}, CO₃R^{A}, OC(O)R^{A}, CH₂OR^{A}, CONR^{A}R^{B}, an optionally substituted C₁-C₆ alkyl, an optionally substituted C₁-C₆ haloalkyl, and an optionally substituted C₁-C₆ heteroalkyl; R⁷ and R⁹ are each independently selected from among hydrogen, halogen, OR^{A}, C₁-C₆ alkyl, C₁-C₆ haloalkyl, C₁-C₆ heteroalkyl, and C₁-C₆ haloheteroalkyl; R⁸ is selected from among NO₂, CN, COR^{A}, CO₂R^{A}, CONR^{A}R^{B}, SOR^{A}, SO₂R^{A}, and SO₂NR^{A}R^{B}; R^{A} and R^{B} are each independently selected from among hydrogen, C₁-C₆ alkyl, C₁-C₆ haloalkyl, and C₁-C₆ heteroalkyl; and Y and Z are each independently selected from among CR^{A}R^{B}, CR^{A}OR^{B}, CO, OCH₂, CH₂O, O, S(O)ₙ, and NR^{A}.

In certain embodiments, R³ and R⁴ are each independently selected from among hydrogen, halogen, C₁-C₆ alkyl, and C₁-C₆ haloalkyl; R⁵ and R⁶ are each independently selected from among hydrogen, halogen, OR^{A}, COR^{A}, CO₂R^{A}, OC(O)R^{A}, CH₂OR^{A}, CONR^{A}R^{B}, an optionally substituted C₁-C₆ alkyl, an optionally substituted C₁-C₆ haloalkyl, and an optionally substituted C₁-C₆ heteroalkyl; R⁷ and R⁹ are each independently selected from among hydrogen, halogen, OR^{A}, C₁-C₆ alkyl, C₁-C₆ haloalkyl, C₁-C₆ heteroalkyl, and C₁-C₆ haloheteroalkyl; R⁸ is selected from among NO₂, CN, COR^{A}, CO₂R^{A}, and CONR^{A}R^{B}; R^{A} and R^{B} are each independently selected from among hydrogen, C₁-C₆ alkyl, and C₁-C₆ haloalkyl; and Y and Z are each independently selected from among CR^{A}R^{B}, CR^{A}OR^{B}, OCH₂, CH₂O, and O.

In certain embodiments, R³ and R⁴ are each hydrogen; R⁵ and R⁶ are each independently selected from among hydrogen, halogen, OR^{A}, COR^{A}, CO₂R^{A}, OC(O)R^{A}, CH₂OR^{A}, CONR^{A}R^{B}, an optionally substituted C₁-C₆ alkyl, an optionally substituted C₁-C₆ haloalkyl, and an optionally substituted C₁-C₆ heteroalkyl; R⁷ and R⁹ are each independently selected from among hydrogen, halogen, OR^{A}, C₁-C₆ alkyl, C₁-C₆ haloalkyl, C₁-C₆ heteroalkyl, and C₁-C₆ haloheteroalkyl; R⁸ is NO₂; R^{A} and R^{B} are each independently selected from among hydrogen, C₁-C₆ alkyl, and C₁-C₆ haloalkyl; and Y and Z are each independently selected from among CR^{A}R^{B}, CR^{A}OR^{B}, CO, OCH₂, CH₂O, and O.

In certain embodiments, the compounds provided herein have a structure of Formula IV:

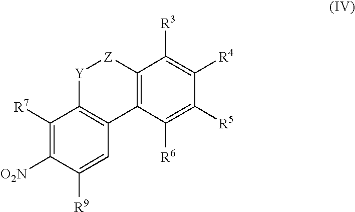

wherein:

R³ and R⁴ are each independently selected from among hydrogen, halogen, C₁-C₆ alkyl, C₁-C₆ haloalkyl, C₁-C₆ heteroalkyl, and C₁-C₆ haloheteroalkyl;

R⁵ and R⁶ are each independently selected from among hydrogen, halogen, OR^{A}, S(O)ₙR^{A}, NR^{A}R^{B}, NR^{A}S(O)ₙR^{B}, COR^{A}, CO₂R^{A}, OC(O)R^{A}, CH₂OR^{A}, CONR^{A}R^{B}, an optionally substituted C₁-C₆ alkyl, an optionally substituted C₁-C₆ haloalkyl, and an optionally substituted C₁-C₆ heteroalkyl; or

R⁵ and R⁶> are linked to form a heterocyclic ring;

R⁷ and R⁹ are each independently selected from among hydrogen, halogen, OR^{A}, C₁-C₆ alkyl, C₁-C₆ haloalkyl, C₁-C₆ heteroalkyl, and C₁-C₆ haloheteroalkyl;

R^{A} and R^{B} are each independently selected from among hydrogen, C₁-C₆ alkyl, C₁-C₆ haloalkyl, and C₁-C₆ heteroalkyl; or

R^{A} and R^{B} are linked to form a ring;

Y and Z are each independently selected from among CR^{A}R^{B}, CR^{A}OR^{B}, CO, OCH₂, CH₂O, O, S(O)ₙ, and NR^{A};

n is selected from among 0, 1, and 2;

provided that, if Y is the same as Z, then R⁴ is not isopropyl;

provided that at least one of R³, R⁴, R⁵, R⁶, and R⁹ is not hydrogen; and

provided that, if Y is CO and Z is O and R⁷ is hydrogen, then R⁹ is not hydrogen.

In certain embodiments, R³ and R⁴ are each independently selected from among hydrogen, halogen, C₁-C₆ alkyl, C₁-C₆ haloalkyl, C₁-C₆ heteroalkyl, and C₁-C₆ haloheteroalkyl. In certain embodiments, R³ and R⁴ are each independently selected from among hydrogen, halogen, C₁-C₆ alkyl, and C₁-C₆ haloalkyl. In certain embodiments, R³ and R⁴ are each hydrogen.

In certain embodiments, R⁵ and R⁶ are each independently selected from among hydrogen, halogen, OR^{A}, COR^{A}, CO₂R^{A}, OC(O)R^{A}, CH₂OR^{A}, CONR^{A}R^{B}, an optionally substituted C₁-C₆ alkyl, an optionally substituted C₁-C₆ haloalkyl, and an optionally substituted C₁-C₆ heteroalkyl. In certain embodiments, R⁵ and R⁶ are each independently selected from among hydrogen, halogen, OR^{A}, COR^{A}, CO₂R^{A}, OC(O)R^{A}, CH₂OR^{A}, CONR^{A}R^{B} an optionally substituted C₁-C₆ alkyl, and an optionally substituted C₁-C₆ haloalkyl. In certain embodiments, R⁵ and R⁶ are each independently selected from among hydrogen, halogen, OR^{A}, COR^{A}, CO₂R^{A}, OC(O)R^{A}, CH₂OR^{A}, and CONR^{A}R^{B}. In certain embodiments, R⁵ and R⁶ are each independently selected from among hydrogen, halogen, OR^{A}, and OC(O)R^{A}.

In certain embodiments, R⁷ and R⁹ are each independently selected from among hydrogen, halogen, OR^{A}, C₁-C₆ alkyl, C₁-C₆ haloalkyl, C₁-C₆ heteroalkyl, and C₁-C₆ haloheteroalkyl. In certain embodiments, R⁷ and R⁹ are each independently selected from among hydrogen, halogen, OR^{A}, C₁-C₆ alkyl, and C₁-C₆ haloalkyl. In certain embodiments, R⁷ and R⁹ are each independently selected from among hydrogen, C₁-C₆ alkyl, and C₁-C₆ haloalkyl. In certain embodiments, R⁷ and R⁹ are each independently selected from among hydrogen and C₁-C₆ alkyl. In certain embodiments. R⁷ and R⁹ are each independently selected from among hydrogen and methyl.

In certain embodiments, R^{A} and R^{B} are each independently selected from among hydrogen, C₁-C₆ alkyl, C₁-C₆ haloalkyl, and C₁-C₆ heteroalkyl. In certain embodiments, R^{A} and R^{B} are each independently selected from among hydrogen, C₁-C₆ alkyl, and C₁-C₆ haloalkyl. In certain embodiments, R^{A} and R^{B} are each independently selected from among hydrogen and C₁-C₆ alkyl. In certain embodiments, R^{A} and R^{B} are each independently selected from among hydrogen and methyl.

In certain embodiments, Y and Z are each independently selected from among CR^{A}R^{B}, CR^{A}OR^{B}, CO, OCH₂, CH₂O, O, S(O)ₙ, and NR^{A}. In certain embodiments, Y and Z are each independently selected from among CR^{A}R^{B}, CR^{A}OR^{B}, CO, OCH₂, CH₂O, and O. In certain embodiments, Y and Z are each independently selected from among CO and O.

In certain embodiments, the compounds provided herein have a structure of Formula V:

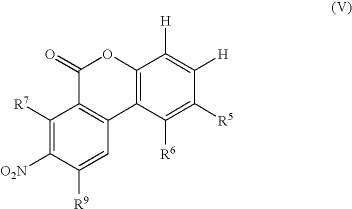

wherein:

R⁵ and R⁶ are each independently selected from among hydrogen, halogen, OR^{A}, S(O)ₙR^{A}, NR^{A}R^{B}, NR^{A}S(O)ₙR^{B}, COR^{A}, CO₂R^{A}, OC(O)R^{A}, CH₂OR^{A}, CONR^{A}R^{B}, an optionally substituted C₁-C₆ alkyl, an optionally substituted C₁-C₆ haloalkyl, and an optionally substituted C₁-C₆ heteroalkyl; or

R⁵ and R⁶ are linked to form a heterocyclic ring;

R⁷ and R⁹ are each independently selected from among hydrogen, halogen, OR^{A}, C₁-C₆ alkyl, C₁-C₆ haloalkyl, C₁-C₆ heteroalkyl, and C₁-C₆ haloheteroalkyl;

R^{A} and R^{B} are each independently selected from among hydrogen, C₁-C₆ alkyl, C₁-C₆ haloalkyl, and C₁-C₆ heteroalkyl; or

R^{A} and R^{B} are linked to form a ring;

n is selected from among 0, 1, and 2; and

provided that R⁷ and R⁹ are not hydrogen at the same time,

In certain embodiments, R⁵ and R⁵ are each independently selected from among hydrogen, halogen, OR^{A}, COR^{A}, CO₂R^{A}, OC(O)R^{A}. CH₃₀R^{A}, CONR^{A}R^{B}, an optionally substituted C₁-C₆ alkyl, an optionally substituted C₁-C₆ haloalkyl, and an optionally substituted C₁-C₆ heteroalkyl. In certain embodiments, R⁵ and R⁶ are each independently selected from among hydrogen, halogen, OR^{A}, and OC(O)R^{A}.

In certain embodiments, R⁷ and R⁹ are each independently selected from among hydrogen, halogen, OR^{A}, C₁-C₆ alkyl, C₁-C₆ haloalkyl, C₁-C₆ heteroalkyl, and C₁-C₆ haloheteroalkyl. In certain embodiments, R⁷ and R⁹ are each independently selected from among hydrogen, halogen, OR^{A}, C₁-C₆ alkyl, and C₁-C₆ haloalkyl. In certain embodiments, R⁷ and R⁹ are each independently selected from among hydrogen, C₁-C₆ alkyl, and C₁-C₆ haloalkyl. In certain embodiments, R⁷ and R⁹ are each independently selected from among hydrogen and C₁-C₆ alkyl. In certain embodiments, R⁷ and R⁹ are each independently selected from among hydrogen and methyl.

In certain embodiments, R^{A} and R^{B} are each independently selected from among hydrogen, C₁-C₆ alkyl, C₁-C₆ haloalkyl, and C₁-C₆ heteroalkyl. In certain embodiments, R^{A} and R^{B} are each independently selected from among hydrogen, C₁-C₆ alkyl, and C₁-C₆ haloalkyl. In certain embodiments, R^{A} and R^{B} are each independently selected from among hydrogen and C₁-C₆ alkyl. In certain embodiments, R^{A} and R^{B} are each independently selected from among hydrogen and methyl.

In certain embodiments, provided herein are compounds of formula II that have the formula:

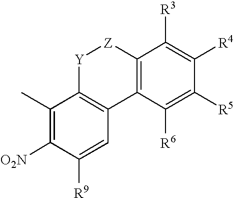

wherein;

R³ and R⁴ are each independently selected from among hydrogen, halogen, C₁-C₆ alkyl, C₁-C₆ haloalkyl, C₁-C₆ heteroalkyl, and C₁-C₆ haloheteroalkyl;

R⁵ and R⁶ are each independently selected from among hydrogen, halogen, OR^{A}, S(O)ₙR^{A}, NR^{A}R^{B}, NR^{A}S(O)ₙR^{B}, COR^{A}, CO₂R^{A}, OC(O)R^{A}, CH₃₀R^{A}, CONR^{A}R^{B}, an optionally substituted C₁-C₆ alkyl, an optionally substituted C₁-C₆ haloalkyl, and an optionally substituted C₁-C₆ heteroalkyl; or R⁵ and R⁶ are linked to form a heterocyclic ring;

R⁹ is selected from among hydrogen, halogen, OR^{A}, C₁-C₆ alkyl, C₁-C₆ haloalkyl, C₁-C₆ heteroalkyl, and C₁-C₆ haloheteroalkyl;

R^{A} and R^{B} are each independently selected from among hydrogen, C₁-C₆ alkyl, C₁-C₆ haloalkyl, and C₁-C₆ heteroalkyl; or R^{A} and R^{B} are linked to form a ring;

Y and Z are each independently selected from among CR^{A}R^{B}, CR^{A}OR^{B}, CO, OCH₂, CH₂O, O, S(O)ₙ, and NR^{A}; and

Y and Z are not the same.

In certain embodiments, compounds provided herein have a structure of Formula II:

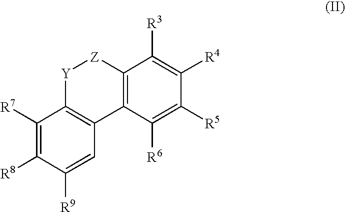

wherein:

R³ and R⁴ are each independently selected from among hydrogen, halogen, C₁-C₆ alkyl, C₁-C₆ haloalkyl, C₁-C₆ heteroalkyl, and C₁-C₆ haloheteroalkyl;

R⁵ is selected from among hydrogen, halogen, OR^{A}, S(O)ₙR^{A}, NR^{A}R^{B}, NR^{A}S(O)ₙR^{B}, COR^{A}, CO₂R^{A}, OC(O)R^{A}, CH₂OR^{A}, CONR^{A}R^{B}, an optionally substituted C₁-C₆ alkyl, an optionally substituted C₁-C₆ haloalkyl, and an optionally substituted C₁-C₆ heteroalkyl;

R⁶ is selected from among halogen, OR^{A}, S(O)ₙR^{A}, NR^{A}R^{B}, NR^{A}S(O)ₙR^{B}, COR^{A}, CO₂R^{A}, OC(O)R^{A}, CH₂OR^{A}, CONR^{A}R^{B}, an optionally substituted C₁-C₆ alkyl, an optionally substituted C₁-C₆ haloalkyl, and an optionally substituted C₁-C₆ heteroalkyl; or

R⁵ and R⁶ are linked to form a heterocyclic ring;

R⁷ and R⁹ are each independently selected from among hydrogen, halogen, OR^{A}, C₁-C₆ alkyl, C₁-C₆ haloalkyl, C₁-C₆ heteroalkyl, and C₁-C₆ haloheteroalkyl;

R⁸ is selected from among NO₂, CN, COR^{A}, CO₂R^{A}, CONR^{A}R^{B}, SOR^{A}, SO₂R^{A}, SO₂NR^{A}R^{B}, C₁-C₆ haloalkyl, and C₁-C₄ haloheteroalkyl;

R^{A} and R^{B} are each independently selected from among hydrogen, C₁-C₆ alkyl, C₁-C₆ haloalkyl, and C₁-C₆ heteroalkyl; or

R^{A} and R^{B} are linked to form a ring;

Y and Z are each independently selected from among CR^{A}R^{B}, CR^{A}OR^{B}, CO, OCH₂, CH₂O, O, S(O)ₙ, and NR^{A}; and

n is selected from among 0, 1, and 2;

provided that, if R⁸ is NO₂ and Y is CO and Z is O and R⁷ is hydrogen, then R⁹ is not hydrogen.

In certain embodiments, the compounds provided herein have a structure of Formula II;

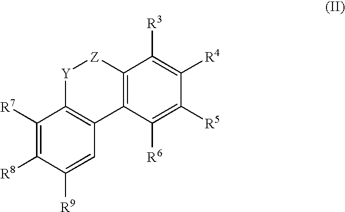

wherein:

R³ and R⁴ are each independently selected from among hydrogen, halogen, C₁-C₆ alkyl, C₁-C₆ haloalkyl, C₁-C₆ heteroalkyl, and C₁-C₆ haloheteroalkyl;

R⁵ and R⁶ are each independently selected from among hydrogen, halogen, OR^{A}, S(O)ₙR^{A}, NR^{A}R^{B}, NR^{A}S(O)ₙR^{B}, COR^{A}, CO₂R^{A}, OC(O)R^{A}, CH₂OR^{A}, CONR^{A}R^{B}, an optionally substituted C₁-C₆ alkyl, an optionally substituted C₁-C₆ haloalkyl, and an optionally substituted C₁-C₆ heteroalkyl; or

R⁵ and R⁶ are linked to form a heterocyclic ring;

R⁷ is selected from among hydrogen, halogen, OR^{A}, C₁-C₆ alkyl, C₁-C₆ haloalkyl, C₁-C₆ heteroalkyl, and C₁-C₆ haloheteroalkyl;

R⁹ is selected from among hydrogen, halogen, OR^{A}, C₁-C₆ alkyl, C₁-C₆ haloalkyl, C₁-C₆ heteroalkyl, and C₁-C₆ haloheteroalkyl;

R⁸ is selected from among NO₂, CN, COR^{A}, CO₂R^{A}, CONR^{A}R^{B}, SOR^{A}, SO₂R^{A}, SO₂NR^{A}R^{B}, C₁-C₆ haloalkyl, and C₁-C₄ haloheteroalkyl;

R^{A} and R^{B} are each independently selected from among hydrogen, C₁-C₆ alkyl, C₁-C₆ haloalkyl, and C₁-C₆ heteroalkyl; or

R^{A} and R^{B} are linked to form a ring;

Y and Z are each independently selected from among CR^{A}R^{B}, CR^{A}OR^{B}, CO, OCH₂, CH₂O, O, S(O)ₙ, and NR^{A};

n is selected from among 0, 1, and 2;

provided that, if R⁸ is NO₂, CN, COR^{A}, CO₃R^{A} and CONR^{A}R^{B}, and Y or Z is CO, and R⁹ is hydrogen, then R⁷ is not hydrogen; and

provided that, if R⁸ is CN, CO₂R^{A} or COR^{A}, and R⁷ is methyl, then one of R⁵, R⁶ or R⁹ is not hydrogen; and

provided that Y and Z are not the same.

In certain embodiments, R³ and R⁴ are each independently selected from among hydrogen, halogen, and C₁-C₆ alkyl. In certain embodiments, R³ and R⁴ are each independently selected from among hydrogen and halogen. In certain embodiments, R³ and R⁴ are each hydrogen.

In certain embodiments, R⁵ and R⁶ are each independently selected from among hydrogen, halogen, OR^{A}, S(O)ₙR^{A}, NR^{A}R^{B}, NR^{A}S(O)ₙR^{B}, COR^{A}, CO₂R^{A}, OC(O)R^{A}, CH₂OR^{A}, CONR^{A}R^{B}, an optionally substituted C₁-C₆ alkyl, an optionally substituted C₁-C₆ haloalkyl, and an optionally substituted C₁-C₆ heteroalkyl. In certain embodiments, R⁵ and R⁶ are each independently selected from among hydrogen, halogen, OR^{A}, S(O)ₙR^{A}, NR^{A}R^{B}, NR^{A}S(O)ₙR^{B}, COR^{A}, CO₂R^{A}, OC(O)R^{A}, CH₂OR^{A}, CONR^{A}R^{B}, and an optionally substituted C₁-C₆ alkyl. In certain embodiments, R⁵ and R⁶ are each independently selected from among hydrogen, halogen, OR^{A}, COR^{A}, CO₂R^{A}, OC(O)R^{A}CH₂OR^{A}, CONR^{A}R^{B}, and an optionally substituted C₁-C₆ alkyl. In certain embodiments, R⁵ and R⁶ are each independently selected from among hydrogen, halogen, OR^{A}, COR^{A}, CO₂R^{A}, OC(O)R^{A}, CH₂OR^{A}, and CONR^{A}R^{B}. In certain embodiments, R⁵ and R⁶ are each independently selected from among hydrogen, OR^{A}, COR^{A}, CO₂R^{A}, OC(O)R^{A}, CH₂OR^{A}, and CONR^{A}R^{B}. In certain embodiments, R⁵ and R⁶ are each independently selected from among hydrogen, OR^{A}, CO₂R^{A} and OC(O)R^{A}.

In certain embodiments, R⁷ is selected from among hydrogen, halogen, OR^{A}, C₁-C₆ alkyl, C₁-C₆ haloalkyl, C₁-C₆ heteroalkyl, and C₁-C₆ haloheteroalkyl. In certain embodiments, R⁷ is selected from among halogen, OR^{A}, C₁-C₆ alkyl, and C₁-C₆ haloalkyl. In certain embodiments, R⁷ is selected from among C₁-C₆ alkyl and C₁-C₆ haloalkyl. In certain embodiments, R⁷ is C₁-C₆ alkyl. In certain embodiments, R⁷ is methyl.

In certain embodiments, R⁹ is selected from among hydrogen, halogen, C₁-C₆ alkyl and C₁-C₆ haloalkyl. In certain embodiments, R⁹ is selected from among hydrogen and C₁-C₆ alkyl. In certain embodiments, R⁹ is hydrogen.

In certain embodiments, R⁸ is selected from among NO₂, CN, COR^{A}, CO₂R^{A}, CONR^{A}R^{B}, SOR^{A}, SO₂R^{A}, and SO₂NR^{A}R^{B}. In certain embodiments, R⁸ is selected from among NO₂, SOR^{A}, SO₂R^{A}, and SO₂NR^{A}R^{B}. In certain embodiments, R⁸ is NO₂.

In embodiments in which two or more of a particular group are present, the identities of those two or more particular groups are selected independently and, thus, can be the same as or different from one another. For example, certain compounds provided herein contain two or more R^{A} groups. The identities of those two or more R^{A} groups are each selected independently. Thus, in certain embodiments, those R^{A} groups are all the same as one another; in certain embodiments, those R^{A} groups are, all different from one another; and in certain embodiments, some of those R^{A} groups are the same as one another and some are different from one another. This independent selection applies to any group that is present in a compound more than once.

In certain embodiments, provided herein is a method for modulating an

activity of an androgen receptor that includes contacting an androgen receptor with a compound of Formula I or Formula II:

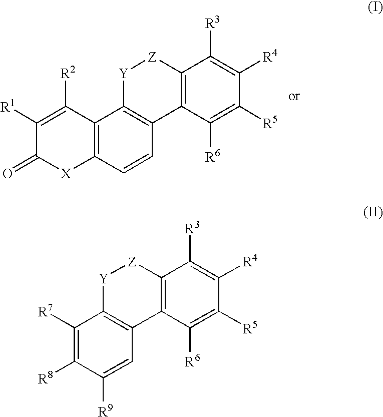

wherein:

R¹ is selected from among hydrogen, F, Cl, C₁-C₄ alkyl, and C₁-C₄ haloalkyl;

R² is selected from among hydrogen, halogen, C₁-C₆ alkyl, C₁-C₆ haloalkyl, C₁-C₆ heteroalkyl, and C₁-C₄ haloheteroalkyl;

R³ and R⁴ are each independently selected from among hydrogen, halogen, C₁-C₆ alkyl, C₁-C₆ haloalkyl, C₁-C₆ heteroalkyl, and C₁-C₆ haloheteroalkyl;

R⁵ and R⁶ are each independently selected from among hydrogen, halogen, OR^{A}, S(O)ₙR^{A}, NR^{A}R^{B}, NR^{A}S(O)ₙR^{B}, COR^{A}, CO₂R^{A}, OC(C)R^{A}, CH₂OR^{A}, CONR^{A}R^{B}, an optionally substituted C₁-C₆ alkyl, an optionally substituted C₁-C₆ haloalkyl, and an optionally substituted C₁-C₆ heteroalkyl; or R⁵ and R⁶ are linked to form a heterocyclic ring;

R⁷ and R⁹ are each independently selected from among hydrogen, halogen, OR^{A}, C₁-C₆ alkyl, C₁-C₆ haloalkyl, C₁-C₆ heteroalkyl, and C₁-C₆ haloheteroalkyl;

R⁸ is selected from among NO₂, CN, COR^{A}, CO₂R^{A}, CONR^{A}R^{B}, SOR^{A}, SO₂R^{A}, SO₂NR^{A}R^{B}, C₁-C₆ haloalkyl, and C₁-C₄ haloheteroalkyl;

R^{A} and R^{B} are each independently selected from among hydrogen, C₁-C₆ alkyl, C₁-C₆ haloalkyl, and C₁-C₆ heteroalkyl; or R^{A} and R^{B} are linked to form a ring;

X is selected from among O, S, and NR^{A};

Y and Z are each independently selected from among CR^{A}R^{B}, CR^{A}OR^{B}, CO, OCH₂, CH₂O, O, S(O)ₙ, and NR^{A}; and

n is selected from among 0, 1, and 2; and

pharmaceutically acceptable salts and prodrugs thereof.

In certain embodiments, provided herein is a method for modulating an activity of an androgen receptor that includes contacting an androgen receptor with a compound of Formula I or formula II;

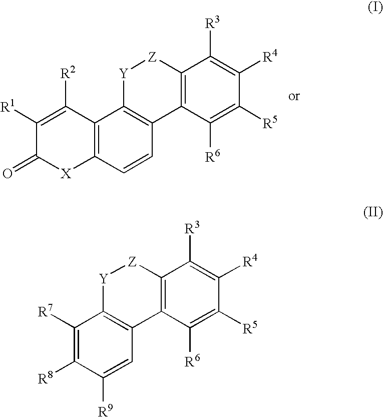

wherein;

R¹ is selected from among hydrogen, F, Cl, C₁-C₄ alkyl, and C₁-C₄ haloalkyl;

R² is selected from among hydrogen, halogen, C₁-C₆ alkyl, C₁-C₆ haloalkyl, C₁-C₆ heteroalkyl, and C₁-C₄ haloheteroalkyl;

R³ and R⁴ are each independently selected from among hydrogen, halogen, C₁-C₆ alkyl, C₁-C₆ haloalkyl, C₁-C₆ heteroalkyl, and C₁-C₆ haloheteroalkyl;

R⁵ and R⁶ are each, independently selected from among hydrogen, halogen, OR^{A}, S(O)ₙR^{A}, NR^{A}R^{B}, NR^{A}S(O)ₙR^{B}, COR^{A}, CO₂R^{A}, OC(O)R^{A}, CH₂OR^{A}, CONR^{A}R^{B}, an optionally substituted C₁-C₆ alkyl, an optionally substituted C₁-C₆ haloalkyl, and an optionally substituted C₁-C₆ heteroalkyl; or R⁵ and R⁶ are linked to form a, heterocyclic ring;

R⁷ and R⁹ are each independently selected from among hydrogen, halogen, OR^{A}, C₁-C₆ alkyl, C₁-C₆ haloalkyl, C₁-C₆ heteroalkyl, and C₁-C₆ haloheteroalkyl;

R⁸ is selected from among NO₂, CN, COR^{A}, CO₂R^{A}, CONR^{A}R^{B}, SOR^{A}, SO₂R^{A}, SO₂NR^{A}R^{B}, C₁-C₆ haloalkyl, and C₁-C₄ haloheteroalkyl;

R^{A} and R^{B} are each independently selected from among hydrogen, C₁-C₆ alkyl, C₁-C₆ haloalkyl, and C₁-C₆ heteroalkyl; or R^{A} and R^{B} are linked to form a ring;

X is selected from among O, S, and NR^{A};

Y and Z are each independently selected from among CR^{A}R^{B}, CR^{A}OR^{B}, CO, OCH₂, CH₂O, O, S(O)ₙ, and NR^{A};

n is selected from among 0, 1, and 2; and

provided that, if X is NH, and Y is CO, and Z is O, and each of R¹, R³, R⁴, and R⁵ is hydrogen, and R² is CH₃, then R⁶ is not OCH₃; and

pharmaceutically acceptable salts and prodrugs thereof.

In certain embodiments, provided herein is a method for modulating an activity of an androgen receptor that includes contacting an androgen receptor with a compound of Formula I:

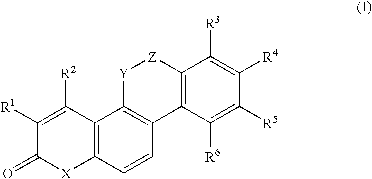

wherein:

R¹ is selected from among hydrogen, F, Cl, C₁-C₄ alkyl, and C₁-C₄ haloalkyl;

R² is selected from among hydrogen, halogen, C₁-C₆ alkyl, C₁-C₆ haloalkyl, C₁-C₆ heteroalkyl, and C₁-C₄ haloheteroalkyl;

R³ and R⁴ are each independently selected from among hydrogen, halogen, C₁-C₆ alkyl, C₁-C₆ haloalkyl, C₁-C₆ heteroalkyl, and C₁-C₆ haloheteroalkyl;

R⁵ and R⁶ are each independently selected from among hydrogen, halogen, OR^{A}, S(O)ₙR^{A}, NR^{A}R^{B}, NR^{A}S(O)ₙR^{B}, COR^{A}, CO₂R^{A}, OC(O)R^{A}, CH₂OR^{A}, CONR^{A}R^{B}, an optionally substituted C₁-C₆ alkyl, an optionally substituted C₁-C₆ haloalkyl, and an optionally substituted C₁-C₆ heteroalkyl; or R⁵ and R⁶ are linked to form a heterocyclic ring;

R^{A} and R^{B} are each independently selected from among hydrogen, C₁-C₆ alkyl, C₁-C₆ haloalkyl, and C₁-C₆ heteroalkyl; or R^{A} and R^{B} are linked to form a ring;

X is selected from among O, S, and NR^{A};

Y and Z are each independently selected from among CR^{A}R^{B}, CR^{A}OR^{B}, CO, OCH₂, CH₂O, O, S(O)ₙ, and NR^{A};

n is selected from among 0, 1, and 2; and

pharmaceutically acceptable salts and prodrugs thereof.

In certain, embodiments, provided herein is a method for modulating an activity of an androgen receptor that includes contacting an androgen receptor with a compound of Formula I or Formula II, wherein:

if X is NH, and Y is CO, and Z is O, and each of R¹, R³, R⁴, and R⁵ is hydrogen, and R² is CH₃, then R⁶ is not OCH₃.

In certain embodiments, provided herein is a method for modulating an

activity of an androgen receptor that includes contacting an androgen receptor with a compound of Formula II:

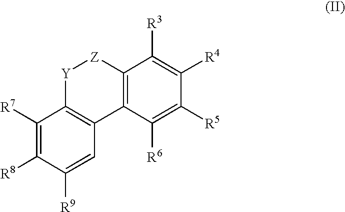

wherein:

R³ and R⁴ are each independently selected from among hydrogen, halogen, C₁-C₆ alkyl, C₁-C₆ haloalkyl, C₁-C₆ heteroalkyl, and C₁-C₆ haloheteroalkyl;

R⁵ and R⁶ are each independently selected from among hydrogen, halogen, OR^{A}, S(O)ₙR^{A}, NR^{A}R^{B}, NR^{A}S(O)ₙR^{B}, COR^{A}, CO₂R^{A}, OC(O)R^{A}, CH₂OR^{A}, CONR^{A}R^{B}, an optionally substituted C₁-C₆ alkyl, an optionally substituted C₁-C₆ haloalkyl, and an optionally substituted C₁-C₆ heteroalkyl; or R⁵ and R⁶ are linked to form a heterocyclic ring;

R⁷ and R⁹ are each independently selected from among hydrogen, halogen, OR^{A}, C₁-C₆ alkyl, C₁-C₆ haloalkyl, C₁-C₆ heteroalkyl, and C₁-C₆ haloheteroalkyl;

R⁸ is selected from among NO₂, CN, COR^{A}, CO₂R^{A}, CONR^{A}R^{B}, SOR^{A}, SO₂R^{A}, SO₃NR^{A}R^{B}, C₁-C₆ haloalkyl, and C₁-C₄ haloheteroalkyl;

R^{A} and R^{B} are each independently selected from among hydrogen, C₁-C₆ alkyl, C₁-C₆ haloalkyl, and C₁-C₆ heteroalkyl; or R^{A} and R^{B} are linked to form a ring;

Y and Z are each independently selected from among CR^{A}R^{B}CR^{A}OR^{B}, CO, OCH₂, CH₂O, O, S(O)ₙ and NR^{A};

n is selected from among 0, 1, and 2; and

pharmaceutically acceptable salts and prodrugs thereof.

In certain embodiments, provided herein is a method for modulating an activity of an androgen receptor that includes contacting an androgen receptor with a compound of Formula I or Formula II, wherein:

provided that, if the compound has a structure of Formula II and Y is the same as Z, then R⁴ is not isopropyl;

provided that, if the compound has a structure of Formula II, at least one of R³, R⁴, R⁵, R⁶, and R⁹ is not hydrogen; and

provided that, if R⁸ is NO₂ and Y is CO and Z is O and R⁷ is hydrogen, then R⁹ is not hydrogen; and

provided that, if R⁸ is CO₂R^{A} or CONR^{A}R^{B}, then R⁴ is not F or R⁹ is not methoxy.

Any combination of the groups described above for the various variables is contemplated herein.

In certain embodiments, provided herein is a compound selected from among:
9-Fluoro-4-methyl-1H-6-oxa-1-aza-chrysene-2,5-dione (Compound 101);
2,2-Dimethyl-propionic acid 10-methoxy-4-methyl-2,5-dioxo-2,5-dihydro-1H-6-oxa-1-aza-chrysen-9-yl ester (Compound 102);
9-Methoxy-4-methyl-1H-6-oxa-1-aza-chrysene-2,5-dione (Compound 103);
2,2-Dimethyl-propionic acid 4-methyl-2,5-dioxo-2,5-dihydro-1H-6-oxa-1-aza-chrysen-10-yl ester (Compound 104);
5-Hydroxy-10-methoxy-4-methyl-1H,5H-6-oxa-1-aza-chrysen-2-one (Compound 105);
5,10-Dimethoxy-4-methyl-1H,5H-6-oxa-1-aza-chrysen-2-one (Compound 106);
(±)-10-Methoxy-4,5-dimethyl-1H,5H-6-oxa-1-aza-chrysen-2-one (Compound 107);

(±)-5,10-Dimethoxy-4,5-dimethyl-1H,5H-6-oxa-1-aza-chrysen-2-one (Compound 108);
10-Methoxy-4-methyl-1H,5H-6-oxa-1-aza-chrysen-2-one (Compound 109);
5-Allyl-10-methoxy-4-methyl-1H,5H-6-oxa-1-aza-chrysen-2-one (Compound 110);
4-Methyl-2,5-dioxo-2,5-dihydro-1H-6-oxa-1-aza-chrysene-10-carboxylic acid methyl ester (Compound 111);
10-Hydroxymethyl-4-methyl-1H,5H-6-oxa-1-aza-chrysen-2-one (Compound 112);
10-Hydroxymethyl-4-trifluoromethyl-1H,5H-6-oxa-1-aza-chrysen-2-one (Compound 113);
5-Hydroxy-10-methoxy-4-trifluoromethyl-1H,5H-6-oxa-1-aza-chrysen-2-one (Compound 114);
10-Hydroxy-4-trifluoromethyl-1H,5H-6-oxa-1-aza-chrysen-2-one (Compound 115);
2,2-Dimethyl-propionic acid 2-oxo-4-trifluoromethyl-2,5-dihydro-1H-6-oxa-1-aza-chrysen-10-yl ester (Compound 116);
9-Hydroxy-10-methoxy-4-methyl-1H-6-oxa-1-aza-chrysene-2,5-dione (Compound 117);
9-Hydroxy-5,10-dimethoxy-4-methyl-1H,5H-6-oxa-1-aza-chrysen-2-one (Compound 118);
9-Hydroxy-10-methoxy-4-methyl-1H,5H-6-oxa-1-aza-chrysen-2-one (Compound 119);
9-Isopropoxy-10-methoxy-4-methyl-1H-6-oxa-1-aza-chrysene-2,5-dione (Compound 120);
9-Ethoxy-10-methoxy-4-methyl-1H-6-oxa-1-aza-chrysene-2,5-dione (Compound 121);
9-Ethoxy-1-ethyl-10-methoxy-4-methyl-1H-6-oxa-1-aza-chrysene-2,5-dione (Compound 122);
4-Methoxy-10-methyl-7,13-dihydro-12-oxa-7-aza-benzo[3,4]cyclohepta[1,2-α]naphthalene-8,11-dione (Compound 123);
10-Hydroxymethyl-4-methyl-1H-6-oxa-1-aza-chrysene-2,5-dione (Compound 124);
10-Hydroxy-4-trifluoromethyl-1H-5-oxa-1-aza-chrysene-2,6-dione (Compound 125);
10-Methoxy-4-trifluoromethyl-1H-5-oxa-1-aza-chrysene-2,6-dione (Compound 126);
10-Methoxy-4-trifluoromethyl-1,6-dihydro-5-oxa-1-aza-chrysen-2-one (Compound 127);
4-Trifluoromethyl-1,6-dihydro-5-oxa-1-aza-chrysen-2-one (Compound 128);
1-Hydroxy-7-methyl-8-nitro-benzo[c]chromen-6-one (Compound 129); and
1-Methoxy-7-methyl-8-nitro-benzo[c]chromen-6-one (Compound 130);
   and pharmaceutically acceptable salts and prodrugs thereof.

In a further aspect are provided pharmaceutical compositions, comprising a therapeutically effective amount of at least one of any of the compounds herein, or a pharmaceutically acceptable salt or solvate thereof, and a pharmaceutically acceptable carrier or diluent.

In certain embodiments, compounds provided herein are selective androgen receptor modulators. In certain embodiments, compounds provided herein are selective androgen receptor agonists. In certain embodiments, compounds provided herein are selective androgen receptor antagonists. In certain embodiments, compounds provided herein are selective androgen receptor partial agonists. In certain embodiments, compounds provided herein are tissue specific selective androgen receptor modulators. In certain embodiments, compounds provided herein are selective androgen receptor binding compounds. In certain embodiments, compounds provided herein are selective androgen receptor reducing compounds. In certain embodiments, compounds provided herein are selective androgen receptor degrading compounds.

In certain embodiments, compounds provided herein are effective for treating one or more androgen receptor mediated diseases or conditions. In certain embodiments, compounds provided herein are effective for treating one or more diseases or conditions including, but not limited to, increase or maintenance of muscle strength and function (e.g., in the elderly); reversal or prevention of frailty or age-related functional decline (“ARFD”) in the elderly (e.g., sarcopenia); treatment of catabolic side effects of glucocorticoids; prevention and/or treatment of reduced bone mass, density or growth (e.g., osteoporosis and osteopenia); treatment of chronic fatigue syndrome (CFS); treatment of chronic myalgia; treatment of acute fatigue syndrome and muscle loss following elective surgery (e.g., post-surgical rehabilitation); accelerating wound healing; accelerating bone fracture repair (such as accelerating the recovery of hip fracture patients); accelerating healing of complicated fractures, e.g. distraction osteogenesis; in joint replacement; prevention of post-surgical adhesion formation; acceleration of tooth repair or growth; maintenance of sensory function (e.g., hearing, sight, olefaction and taste); treatment of periodontal disease; treatment of wasting secondary to fractures and wasting in connection with chronic obstructive pulmonary disease (COPD), chronic liver disease, AIDS, weightlessness, cancer cachexia, burn and trauma recovery, chronic catabolic state (e.g., coma), eating disorders (e.g., anorexia) and chemotherapy; treatment of cardiomyopathy; treatment of thrombo-cytopenia; treatment of growth retardation in connection with Crohn's disease; treatment of short bowel syndrome; treatment of irritable bowel syndrome; treatment of inflammatory bowel disease; treatment of Crohn's disease and ulcerative colitis; treatment of complications associated with transplantation; treatment of physiological short stature including growth hormone deficient children and short stature associated with chronic illness; treatment of obesity and growth retardation associated with obesity; treatment of anorexia (e.g., associated with cachexia or aging); treatment of hypercortisolism and Cushing's syndrome; treatment of Paget's disease; treatment of osteoarthritis; induction of pulsatile growth hormone release; treatment of osteochondrodysplasias; treatment of depression, nervousness, irritability and stress; treatment of reduced mental energy and low self-esteem (e.g., motivation/assertiveness); improvement of cognitive function (e.g., the treatment of dementia, including Alzheimer's disease and short term memory loss); treatment of catabolism in connection with pulmonary dysfunction and ventilator dependency; treatment of cardiac dysfunction (e.g., associated with valvular disease, myocardial infarction, cardiac hypertrophy or congestive heart failure); lowering blood pressure; protection against ventricular dysfunction or prevention of reperfusion events; treatment of adults in chronic dialysis; reversal or slowing of the catabolic state of aging; attenuation or reversal of protein catabolic responses following trauma (e.g., reversal of the catabolic state associated with surgery, congestive heart failure, cardiac myopathy, burns, cancer, COPD, etc.); reducing cachexia and protein loss due to chronic illness such as cancer or AIDS; treatment of hyperinsulinemia including nesidioblastosis; treatment of immunosuppressed patients; treatment of wasting in connection with multiple sclerosis or other neurodegenerative disorders; promotion of myelin repair; maintenance of skin thickness; treatment of metabolic homeostasis and renal homeostasis (e.g., in the frail elderly); stimulation of osteoblasts, bone remodeling and cartilage growth; regulation of food intake; treatment of insulin resistance, including NIDDM, in mammals (e.g., humans); treatment of insulin resistance in the heart; improvement of sleep quality and correction of the relative hyposomatotropism of senescence due to high increase in REM sleep and a decrease in REM latency; treatment of hypothermia; treatment of congestive heart failure; treatment of lipodystrophy (e.g., in patients taking HIV or AIDS therapies such as protease inhibitors); treatment of muscular atrophy (e.g., due to physical inactivity, bed rest or reduced weight-bearing conditions); treatment of musculoskeletal impairment (e.g., in the elderly); improvement of the overall pulmonary function; treatment of sleep disorders; treatment of the catabolic state of prolonged critical illness; treatment of hirsutism, acne, seborrhea, androgenic alopecia, anemia, hyperpilosity, benign prostate hypertrophy, adenomas and neoplasms of the prostate (e.g., advanced metastatic prostate cancer) and malignant tumor cells containing the androgen receptor, such as is the case for breast, brain, skin, ovarian, bladder, lymphatic, liver and kidney cancers; treatment of cancers of the skin, pancreas, endometrium, lung and colon; treatment of osteosarcoma; treatment of hypercalcemia of malignancy; treatment of metastatic bone disease; treatment of spermatogenesis, endometriosis and polycystic ovary syndrome; counteracting preeclampsia, eclampsia of pregnancy and preterm labor; treatment of premenstrual syndrome; treatment of vaginal dryness; treatment of age related decreased testosterone levels in men, male menopause, hypogonadism, male hormone replacement, male and female sexual dysfunction (e.g., erectile dysfunction, decreased sex drive, sexual well-being, decreased libido), male and female contraception, hair loss, Reaven's Syndrome and the enhancement of bone and muscle performance/strength.

In certain embodiments, compounds provided herein are effective for treating one or more of acne, male-pattern baldness, wasting diseases, hirsutism, hypogonadism, osteoporosis, infertility, impotence, and cancer.

In certain embodiments, compounds provided herein are effective for treating one or more of wasting diseases, hypogonadism, osteoporosis, infertility, impotence, and cancer. In certain embodiments, compounds provided herein are effective for treating one or more of wasting diseases, hypogonadism, osteoporosis, infertility, impotence, and breast cancer. In certain embodiments, compounds provided herein are effective for treating one or more of acne, male-pattern baldness, hirsutism and cancer. In certain embodiments, compounds provided herein are effective for treating one or more of acne, male-pattern baldness, hirsutism, prostatic hyperplasia and prostate cancer.

In certain embodiments, compounds provided herein are effective for treating prostate cancer. In certain embodiments, compounds provided herein are effective for treating androgen dependant prostate cancer. In certain embodiments, compounds provided herein are effective for treating androgen independent prostate cancer. In certain embodiments, compounds provided herein are effective for treating androgen independent androgen receptor dependent prostate cancer.

In certain embodiments, provided herein are methods for modulating an activity of an androgen receptor by contacting an androgen receptor with at least one compound provided herein. In certain such embodiments, the androgen receptor is in a cell.

In certain embodiments, provided herein are methods for decreasing the number of functional androgen receptors present in a cell by contacting an androgen receptor with at least one compound provided herein.

In certain embodiments, provided herein are methods for identifying a compound that is capable of modulating an activity of an androgen receptor and/or decreasing the number of functional androgen receptors by contacting a cell expressing an androgen receptor with a compound provided herein and monitoring an effect of the compound upon the cell.

In certain embodiments, provided herein are methods for treating a patient by administering to the patient a compound provided herein. In certain embodiments, provided herein are methods for treating a patient by identifying a patient in need of such a compound and administering to the patient a compound provided herein. In certain embodiments, the methods provided herein are for increase or maintenance of muscle strength and function (e.g., in the elderly); reversal or prevention of frailty or age-related functional decline (“ARFD”) in the elderly (e.g., sarcopenia); treatment of catabolic side effects of glucocorticoids; prevention and/or treatment of reduced bone mass, density or growth (e.g., osteoporosis and osteopenia); treatment of chronic fatigue syndrome (CFS); treatment of chronic myalgia; treatment of acute fatigue syndrome and muscle loss following elective surgery (e.g., post-surgical rehabilitation); accelerating wound healing; accelerating bone fracture repair (such as accelerating the recovery of hip fracture patients); accelerating healing of complicated fractures, e.g., distraction osteogenesis; in joint replacement; prevention of post-surgical adhesion formation; acceleration of tooth repair or growth; maintenance of sensory function (e.g., hearing, sight, olefaction and taste); treatment of periodontal disease; treatment of wasting secondary to fractures and wasting in connection with chronic obstructive pulmonary disease (COPD), chronic liver disease, AIDS, weightlessness, cancer cachexia, burn and trauma recovery, chronic catabolic state (e.g., coma), eating disorders (e.g., anorexia) and chemotherapy; treatment of cardiomyopathy; treatment of thrombocytopenia; treatment of growth retardation in connection with Crohn's disease; treatment of short bowel syndrome; treatment of irritable bowel syndrome; treatment of inflammatory bowel disease; treatment of Crohn's disease and ulcerative colitis; treatment of complications associated with transplantation; treatment of physiological short stature including growth hormone deficient children and short stature associated with chronic illness; treatment of obesity and growth retardation associated with obesity; treatment of anorexia (e.g., associated with cachexia or aging); treatment of hypercortisolism and Cushing's syndrome; treatment of Paget's disease; treatment of osteoarthritis; induction of pulsatile growth hormone release; treatment of osteochondrodysplasias; treatment of depression, nervousness, irritability and stress; treatment of reduced mental energy and low self-esteem (e.g., motivation/assertiveness); improvement of cognitive function (e.g., the treatment of dementia, including Alzheimer's disease and short term memory loss); treatment of catabolism in connection with pulmonary dysfunction and ventilator dependency; treatment of cardiac dysfunction (e.g., associated with valvular disease, myocardial infarction, cardiac hypertrophy or congestive heart failure); lowering blood pressure; protection against ventricular dysfunction or prevention of reperfusion events; treatment of adults in chronic dialysis; reversal or slowing of the catabolic state of aging; attenuation or reversal of protein catabolic responses following trauma (e.g., reversal of the catabolic state associated with surgery, congestive heart failure, cardiac myopathy, burns, cancer, COPD, etc.); reducing cachexia and protein loss due to chronic illness such as cancer or AIDS; treatment of hyperinsulinemia including nesidioblastosis; treatment of immunosuppressed patients; treatment of wasting in connection with multiple sclerosis or other neurodegenerative disorders; promotion of myelin repair; maintenance of skin thickness; treatment of metabolic homeostasis and renal homeostasis (e.g., in the frail elderly); stimulation of osteoblasts, bone remodeling and cartilage growth; regulation of food intake; treatment of insulin resistance, including NIDDM, in mammals (e.g., humans); treatment of insulin resistance in the heart; improvement of sleep quality and correction of the relative hyposomatotropism of senescence due to high increase in REM sleep and a decrease in REM latency; treatment of hypothermia; treatment of congestive heart failure; treatment of lipodystrophy (e.g., in patients taking HIV or AIDS therapies such as protease inhibitors); treatment of muscular atrophy (e.g., due to physical inactivity, bed rest or reduced weight-bearing conditions); treatment of musculoskeletal impairment (e.g., in the elderly); improvement of the overall pulmonary function; treatment of sleep disorders; treatment of the catabolic state of prolonged critical illness; treatment of hirsutism, acne, seborrhea, androgenic alopecia, anemia, hyperpilosity, benign prostate hypertrophy, adenomas and neoplasms of the prostate (e.g., advanced metastatic prostate cancer) and malignant tumor cells containing the androgen receptor, such as is the case for breast, brain, skin, ovarian, bladder, lymphatic, liver and kidney cancers; treatment of cancers of the skin, pancreas, endometrium, lung and colon; treatment of osteosarcoma; treatment of hypercalcemia of malignancy; treatment of metastatic bone disease; treatment of spermatogenesis, endometriosis and polycystic ovary syndrome; counteracting preeclampsia, eclampsia of pregnancy and preterm labor; treatment of premenstrual syndrome; treatment of vaginal dryness; treatment of age related decreased testosterone levels in men, male menopause, hypogonadism, male hormone replacement, male and female sexual dysfunction (e.g., erectile dysfunction, decreased sex drive, sexual well-being, decreased libido), male and female contraception, hair loss, Reaven's Syndrome and the enhancement of bone and muscle performance/strength.

In certain of such embodiments, the patient has a condition selected from among acne, male-pattern baldness, wasting diseases, hirsutism, hypogonadism, osteoporoses, infertility, impotence, and cancer.

In certain embodiments, the methods provided herein are for treating a condition including, but not limited to, prostate cancer. In certain such embodiments, the prostate cancer is androgen independent prostate cancer. In certain embodiments, the prostate cancer is androgen independent androgen receptor dependant prostate cancer.

Pharmaceutical compositions formulated for administration by an appropriate route and means containing effective concentrations of one or more of the compounds provided herein, or pharmaceutically acceptable derivatives thereof, that deliver amounts effective for the treatment, prevention, or amelioration of one or more symptoms of diseases or disorders that are modulated or otherwise affected by androgen receptor activity, or in which androgen receptor activity is implicated, are also provided. The effective amounts and concentrations are effective for ameliorating any of the symptoms of any of the diseases or disorders.

In certain embodiments, provided herein is a pharmaceutical composition containing: i) a physiologically acceptable carrier, diluent, and/or excipient; and ii) one or more compounds provided herein.

Articles of manufacture containing packaging material, within the packaging material a compound or composition, or pharmaceutically acceptable derivative thereof, which is effective for modulating the activity of androgen receptor, or for treatment, prevention or amelioration of one or more symptoms of androgen receptor mediated diseases or disorders, or diseases or disorders in which androgen receptor activity is implicated, and a label that indicates that the compound or composition, or pharmaceutically acceptable derivative thereof, is used for modulating the activity of androgen receptor, or for treatment, prevention or amelioration of one or more symptoms of androgen receptor mediated diseases or disorders, or diseases or disorders in which androgen receptor activity is implicated, are provided.

### DETAILED DESCRIPTION

### A. Definitions

Unless defined otherwise, all technical and scientific terms used herein have the same meaning as is commonly understood by one of skill in the art to which claimed subject matter belongs. All patents, patent applications, published materials referred to throughout the entire disclosure herein, unless noted otherwise, are incorporated by reference in their entirety. In the event that there are a plurality of definitions for terms herein, those in this section prevail.

It is to be understood that the foregoing general description and the following detailed description are exemplary and explanatory only and are not restrictive of any subject matter claimed. In this application, the use of the singular includes the plural unless specifically stated otherwise. In this application, the use of “or” means “and/or” unless stated otherwise. Furthermore, use of the term “including” as well as other forms, such as “includes,” and “included,” is not limiting.

The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

As used herein, the abbreviations for any substituent, protective groups, amino acids and other compounds are, unless indicated otherwise, in accord with their common usage, recognized abbreviations, or the IUPAC-IUB Commission on Biochemical Nomenclature (see, (1972) Biochem. 11:942). For example, the abbreviations Me, Et, Pr, i-Pr (or ⁱPr), Bu, t-Bu (or ^{t}Bu), etc. refers to the groups methyl, ethyl, propyl, isopropyl, butyl, tert-butyl, etc.

Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients. Standard techniques can be used for recombinant DNA, oligonucleotide synthesis, and tissue culture and transformation (e.g., electroporation, lipofection). Reactions and purification techniques can be performed e.g., using kits of manufacturer's specifications or as commonly accomplished in the art or as described herein. The foregoing techniques and procedures can be generally performed by conventional methods well known in the art and as described in various general and more specific references that are cited and discussed throughout the present specification. See e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose.

As used herein, the term “selective binding compound” refers to a compound that selectively binds to any portion of one or more target receptors.

As used herein, the term “selective androgen receptor binding compound” refers to a compound that selectively binds to any portion of a androgen receptor.

As used herein, the term “selective androgen receptor binding compound” refers to a compound that selectively binds to any portion of an androgen receptor.

As used herein, the term “selective androgen receptor reducing compound” refers to a compound, the presence of which results in a decrease in the number of functional androgen receptors in a cell. In certain embodiments, the presence of a selective androgen receptor reducing compound results in a decrease of at least 5%, 10%, 20%, 30%, 40%, 50%, 60%, 70%, 80%, 90%, 95%, 98%, or 99% of the functional androgen receptors in a cell.

As used herein, the term “functional androgen receptor” refers to an androgen receptor that is capable of performing at least one activity associated with intact or native androgen receptors.

As used herein, the term “selective androgen receptor degrading compound” refers to a compound, the presence of which results in degradation of androgen receptors in a cell. In certain embodiments, the presence of a selective androgen receptor degrading compound results in degradation of at least 5%, 10%, 20%, 30%, 40%, 50%, 60%, 70%, 80%, 90%, 95%, 98%, or 99% of the androgen receptors in a cell. In certain embodiments, selective androgen receptor degrading compounds destabilize androgen receptors resulting in their degradation.

As used herein, the term “selectively binds” refers to the ability of a selective binding compound to bind to a target receptor with greater affinity than it binds to a non-target receptor. In certain embodiments, specific binding refers to binding to a target with an affinity that is at least 10, 50, 100, 250, 500, 1000 or more times greater than the affinity for a non-target.

As used herein, the term “target receptor” refers to a molecule or a portion of a receptor capable of being bound by a selective binding compound. In certain embodiments, a target receptor is an androgen receptor.

As used herein, the terms “treating” or “treatment” encompass either or both responsive and prophylaxis measures, e.g., designed to inhibit, slow or delay the onset of a symptom of a disease or disorder, achieve a full or partial reduction of a symptom or disease state, and/or to alleviate, ameliorate, lessen, or cure a disease or disorder and/or its symptoms.

As used herein, amelioration of the symptoms of a particular disorder by administration of a particular compound or pharmaceutical composition refers to any lessening of severity, delay in onset, slowing of progression, or shortening of duration, whether permanent or temporary, lasting or transient that can be attributed to or associated with administration of the compound or composition.

As used herein, the term “modulator” refers to a compound that alters an activity of a molecule. For example, a modulator can cause an increase or decrease in the magnitude of a certain activity of a molecule compared to the magnitude of the activity in the absence of the modulator. In certain embodiments, a modulator is an inhibitor, which decreases the magnitude of one or more activities of a molecule. In certain embodiments, an inhibitor completely prevents one or more activities of a molecule. In certain embodiments, a modulator is an activator, which increases the magnitude of at least one activity of a molecule. In certain embodiments the presence of a modulator results in an activity that does not occur in the absence of the modulator.

As used herein, the term “selective modulator” refers to a compound that selectively modulates a target activity.

As used herein, the term “selective androgen receptor modulator” refers to a compound that selectively modulates at least one activity associated with an androgen receptor.

As used herein, the term “selectively modulates” refers to the ability of a selective modulator to modulate a target activity to a greater extent than it modulates a non-target activity. In certain embodiments the target activity is selectively modulated by, for example about 2 fold up to more that about 500 fold, in some embodiments, about 2, 5, 10, 50, 100, 150, 200, 250, 300, 350, 400, 450 or more than 500 fold.

As used herein, the term “target activity” refers to a biological activity capable of being modulated by a selective modulator. Certain exemplary target activities include, but are not limited to, binding affinity, signal transduction, enzymatic activity, tumor growth, inflammation or inflammation-related processes, and amelioration of one or more symptoms associated with a disease or condition.

As used herein, the term “receptor mediated activity” refers to any biological activity that results, either directly or indirectly, from binding of a ligand to a receptor.

As used herein, the term “agonist” refers to a compound, the presence of which results in a biological activity of a receptor that is the same as the biological activity resulting from the presence of a naturally occurring ligand for the receptor.

As used herein, the term “partial agonist” refers to a compound the presence of which results in a biological activity of a receptor that is of the same type as that resulting from the presence of a naturally occurring ligand for the receptor, but of a lower magnitude.

As used herein, the term “antagonist” refers to a compound, the presence of which results in a decrease in the magnitude of a biological activity of a receptor. In certain embodiments, the presence of an antagonist results in complete inhibition of a biological activity of a receptor.

As used herein, the IC₅₀ refers to an amount, concentration or dosage of a particular test compound that achieves a 50% inhibition of a maximal response, such as modulation of androgen receptor activity, in an assay that measures such response.

As used herein, EC₅₀ refers to a dosage, concentration or amount of a particular test compound that elicits a dose-dependent response at 50% of maximal expression of a particular response that is induced, provoked or potentiated by the particular test compound.

As used herein, C₁-Cₓ includes C₁-C₂, C₁-C₃ . . . C₁-Cₓ.

As used herein, the term “alkyl” alone or in combination refers to a straight, branched, or cyclic hydrocarbon chain containing at least one carbon atom. An alkyl group can be a “saturated alkyl,” which means that it does not contain any alkene or alkyne groups. An alkyl group can be an “unsaturated alkyl,” which means that it contains at least one alkene or alkyne group. In certain embodiments, alkyls are optionally substituted.

In certain embodiments, an alkyl contains 1 to 20 carbon atoms (whenever it appears herein, a numerical range such as “1 to 20” refers to each integer in the given range; e.g., “1 to 20 carbon atoms” means that an alkyl group can contain only 1 carbon atom, 2 carbon atoms, 3 carbon atoms, etc., up to and including 20 carbon atoms, although the term “alkyl” also includes instances where no numerical range of carbon atoms is designated). An alkyl can be designated as “C₁-C₄ alkyl” or similar designations. By way of example only, “C₁-C₄ alkyl” indicates an alkyl having one, two, three, or four carbon atoms, i.e., the alkyl is selected from among methyl, ethyl, propyl, iso-propyl, n-butyl, iso-butyl, sec-butyl, and -butyl. Thus C₁-C₄ includes C₁-C₂ and C₁-C₃ alkyl. Alkyls can be substituted or unsubstituted. Alkyls include, but are not limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like, each of which can be optionally substituted.

As used herein, the term “alkenyl” alone or in combination refers to an alkyl group containing at least one carbon-carbon double bond. In certain embodiments, alkenyls are optionally substituted.

As used herein, the term “alkynyl” alone or in combination refers to an alkyl group containing at least one carbon-carbon triple bond. In certain embodiments, alkynyls are optionally substituted.

As used herein, the term “non-cyclic alkyl” refers to an alkyl that is not cyclic (i.e., a straight or branched chain containing at least one carbon atom). Non-cyclic alkyls can be fully saturated or can contain non-cyclic alkenes and/or alkynes. Non-cyclic alkyls can be optionally substituted.

As used herein, the term “haloalkyl” alone or in combination refers to an alkyl in which at least one hydrogen atom is replaced with a halogen atom. In certain of the embodiments in which two or more hydrogen atoms are replaced with halogen atoms, the halogen atoms are all the same as one another. In certain of such embodiments, the halogen atoms are not all the same as one another. Certain haloalkyls are saturated haloalkyls, which do not include any carbon-carbon double bonds or any carbon-carbon triple bonds. Certain haloalkyls are haloalkenes, which include one or more carbon-carbon double bonds. Certain haloalkyls are haloalkynes, which include one or more carbon-carbon triple bonds. In certain embodiments, haloalkyls are optionally substituted.

As used herein, the term “heteroalkyl” alone or in combination refers to a group containing an alkyl and one or more heteroatoms. The point of attachment of the heteroalkyl radical is through a carbon atom of the heteroalkyl radical. Certain heteroalkyls are saturated heteroalkyls, which do not contain any carbon-carbon double bonds or any carbon-carbon triple bonds. Certain heteroalkyls are heteroalkenes, which include at least one carbon-carbon double bond. Certain heteroalkyls are heteroalkynes, which include at least one carbon-carbon triple bond. Certain heteroalkyls are acylalkyls, in which the one or more heteroatoms are within an alkyl chain. Examples of heteroalkyls include, but are not limited to, CH₃C(═O)CH₂—, CH₃C(═O)CH₂CH₂—, CH₃CH₂C(═O)CH₂CH₂—, CH₃C(═O)CH₂CH₂CH₂—, CH₃OCH₂CH₂—, CH₃OC(═O)CH₂—, CH₃NHCH₂—, and the like. In certain embodiments, heteroalkyls are optionally substituted.

As used herein, the term “heterohaloalkyl” alone or in combination refers to a heteroalkyl in which at least one hydrogen atom is replaced with a halogen atom. In certain embodiments, heteroalkyls are optionally substituted.

As used herein, the term “ring” refers to any covalently closed structure. Rings include, for example, carbocycles (e.g., aryls and cycloalkyls), heterocycles (e.g., heteroaryls and non-aromatic heterocycles), aromatics (e.g., aryls and heteroaryls), and non-aromatics (e.g., cycloalkyls and non-aromatic heterocycles). Rings can be optionally substituted. Rings can form part of a ring system.

As used herein, the term “ring system” refers to two or more rings, wherein two or more of the rings are fused. The term “fused” refers to structures in which two or more rings share one or more bonds.

As used herein, the term “carbocycle” refers to a ring, wherein each of the atoms forming the ring is a carbon atom. Carbocylic rings can be formed by three, four, five, six, seven, eight, nine, or more than nine carbon atoms. Carbocycles can be optionally substituted.

As used herein, the term “heterocycle” refers to a ring wherein at least one atom forming the ring is a carbon atom and at least one atom forming the ring is a heteroatom. Heterocyclic rings can be formed by three, four, five, six, seven, eight, nine, or more than nine atoms. Any number of those atoms can be heteroatoms (i.e., a heterocyclic ring can contain one, two, three, four, five, six, seven, eight, nine, or more than nine heteroatoms, provided that at lease one atom in the ring is a carbon atom). Herein, whenever the number of carbon atoms in a heterocycle is indicated (e.g., C₁-C₆ heterocycle), at least one other atom (the heteroatom) must be present in the ring. Designations such as “C₁-C₆ heterocycle” refer only to the number of carbon atoms in the ring and do not refer to the total number of atoms in the ring. It is understood that the heterocylic ring can have additional heteroatoms in the ring. Designations such as “4-6 membered heterocycle” refer to the total number of atoms that are contained in the ring (i.e., a four, five, or six membered ring, in which at least one atom is a carbon atom, at least one atom is a heteroatom and the remaining two to four atoms are either carbon atoms or heteroatoms). In heterocycles comprising two or more heteroatoms, those two or more heteroatoms can be the same or different from one another. Heterocycles can be optionally substituted. Binding to a heterocycle can be at a heteroatom or via a carbon atom. Examples of heterocycles include, but are not limited to the following:

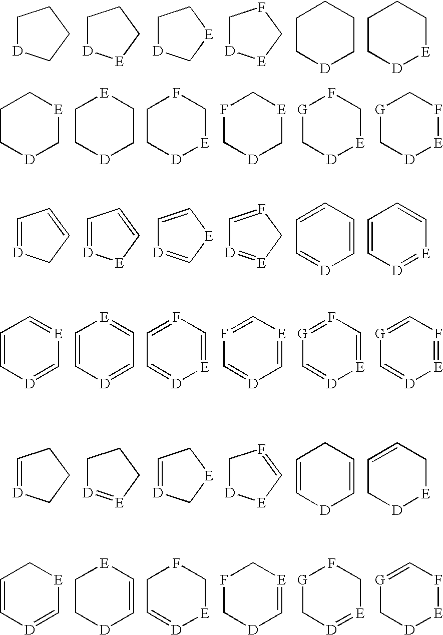

wherein D, E, F, and G independently represent a heteroatom. Each of D, E, F, and G can be the same or different from one another.

As used herein, the term “heteroatom” refers to an atom other than carbon or hydrogen. Heteroatoms are typically independently selected from among oxygen, sulfur, nitrogen, and phosphorus, but are not limited to those atoms. In embodiments in which two or more heteroatoms are present, the two or more heteroatoms can all be the same as one another, or some or all of the two or more heteroatoms can each be different from the others.

As used herein, the term “aromatic” refers to a planar ring having a delocalized π-electron system containing 4n+2 π electrons, where n is an integer. Aromatic rings can be formed by five, six, seven, eight, nine, or more than nine atoms. Aromatics can be optionally substituted. Examples of aromatic groups include, but are not limited to phenyl, naphthalenyl, phenanthrenyl, anthracenyl, tetralinyl, fluorenyl, indenyl, and indanyl. The term aromatic includes, for example, benzenoid groups, connected via one of the ring-forming carbon atoms, and optionally carrying one or more substituents selected from among an aryl, a heteroaryl, a cycloalkyl, a non-aromatic heterocycle, a halo, a hydroxy, an amino, a cyano, a nitro, an alkylamido, an acyl, a C₁-C₆ alkoxy, a C₁-C₆ alkyl, a C₁-C₆ hydroxyalkyl, a C₁-C₆ aminoalkyl, a C₁-C₆ alkylamino, an alkylsulfenyl, an alkylsulfinyl, an alkylsulfonyl, an sulfamoyl, or a trifluoromethyl. In certain embodiments, an aromatic group is substituted at one or more of the para, meta, and/or ortho positions. Examples of aromatic groups containing substitutions include, but are not limited to, phenyl, 3-halophenyl, 4-halophenyl, 3-hydroxyphenyl, 4-hydroxyphenyl, 3-aminophenyl, 4-aminophenyl, 3-methylphenyl, 4-methylphenyl, 3-methoxyphenyl, 4-methoxyphenyl, 4-trifluoromethoxyphenyl, 3-cyanophenyl, 4-cyanophenyl, dimethylphenyl, naphthyl, hydroxynaphthyl, hydroxymethylphenyl, (trifluoromethyl)phenyl, alkoxyphenyl, 4-morpholin-4-ylphenyl, 4-pyrrolidin-1-ylphenyl, 4-pyrazolylphenyl, 4-triazolylphenyl, and 4-(2-oxopyrrolidin-1-yl)phenyl.

As used herein, the term “aryl” refers to an aromatic ring wherein each of the atoms forming the ring is a carbon atom. Aryl rings can be formed by three, four, five, six, seven, eight, nine, or more than nine carbon atoms. Aryl groups can be optionally substituted.

As used herein, the term “heteroaryl” refers to an aromatic ring in which at least one atom forming the aromatic ring is a heteroatom. Heteroaryl rings can be formed by three, four, five, six, seven, eight, nine and more than nine atoms. Heteroaryl groups can be optionally substituted. Examples of heteroaryl groups include, but are not limited to, aromatic C₃-C₈ heterocyclic groups containing one oxygen or sulfur atom or up to four nitrogen atoms, or a combination of one oxygen or sulfur atom and up to two nitrogen atoms, and their substituted as well as benzo- and pyrido-fused derivatives, for example, connected via one of the ring-forming carbon atoms. In certain embodiments, heteroaryl groups are optionally substituted with one or more substituents, independently selected from among halo, hydroxy, amino, cyano, nitro, alkylamido, acyl, C₁-C₆-alkoxy, C₁-C₆-alkyl, C₁-C₆-hydroxyalkyl, C₁-C₆-aminoalkyl, C₁-C₆-alkylamino, alkylsulfenyl, alkylsulfinyl, alkyl sulfonyl, sulfamoyl, or trifluoromethyl. Examples of heteroaryl groups include, but are not limited to, unsubstituted and mono- or di-substituted derivatives of furan, benzofuran, thiophene, benzothiophene, pyrrole, pyridine, indole, oxazole, benzoxazole, isoxazole, benzisoxazole, thiazole, benzothiazole, isothiazole, imidazole, benzimidazole, pyrazole, indazole, tetrazole, quinoline, isoquinoline, pyridazine, pyrimidine, purine and pyrazine, furazan, 1,2,3-oxadiazole, 1,2,3-thiadiazole, 1,2,4-thiadiazole, triazole, benzotriazole, pteridine, phenoxazole, oxadiazole, benzopyrazole, quinolizine, cinnoline, phthalazine, quinazoline, and quinoxaline. In some embodiments, heteroaryl groups are optionally substituted with one or more substituents, independently selected from among halo, hydroxy, cyano, O—C₁-C₆-alkyl, C₁-C₆-alkyl, hydroxy-C₁-C₆-alkyl, and amino-C₁-C₆-alkyl.

As used herein, the term “non-aromatic ring” refers to a ring that does not have a delocalized 4n+2 π-electron system.

As used herein, the term “cycloalkyl” refers to a group containing a non-aromatic ring wherein each of the atoms forming the ring is a carbon atom. Cycloalkyls can be formed by three, four, five, six, seven, eight, nine, or more than nine carbon atoms. Cycloalkyls can be optionally substituted. In certain embodiments, a cycloalkyl contains one or more unsaturated bonds. Examples of cycloalkyls include, but are not limited to, cyclopropane, cyclobutane, cyclopentane, cyclopentene, cyclopentadiene, cyclohexane, cyclohexene, 1,3-cyclohexadiene, 1,4-cyclohexadiene, cycloheptane, and cycloheptene.

As used herein, the term “non-aromatic heterocycle” refers to a non-aromatic ring wherein one or more atoms forming the ring is a heteroatom. Non-aromatic heterocyclic rings can be formed by three, four, five, six, seven, eight, nine, or more than nine atoms. Non-aromatic heterocycles can be optionally substituted. In certain embodiments, non-aromatic heterocycles contain one or more carbonyl or thiocarbonyl groups such as, for example, oxo- and thio-containing groups. Examples of non-aromatic heterocycles include, but are not limited to, lactams, lactones, cyclic imides, cyclic thioimides, cyclic carbamates, tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane.

As used herein, the term “arylalkyl” alone or in combination, refers to an alkyl substituted with an aryl that can be optionally substituted.

As used herein, the term “heteroarylalkyl” alone or in combination, refers to an alkyl substituted with a heteroaryl that can be optionally substituted.

As used herein, the term “halogen” or “halide” or “halo” refers to F, Cl, Br or I.

As used herein, the substituent “R” appearing by itself and without a number designation refers to a substituent selected from among alkyl, cycloalkyl, aryl, heteroaryl (bonded through a ring carbon) and non-aromatic heterocycle (bonded through a ring carbon).

As used herein, the term “O-carboxy” refers to a group of formula RC(═O)O—.

As used herein, the term “C-carboxy” refers to a group of formula —C(═O)OR.

As used herein, the term “acetyl” refers to a group of formula —C(═O)CH₃.

As used herein, the term “trihalomethanesulfonyl” refers to a group of formula X₃CS(═O)₂— where X is a halogen.

As used herein, the term “cyano” refers to a group of formula —CN.

As used herein, the term “isocyanato” refers to a group of formula —NCO.

As used herein, the term “thiocyanato” refers to a group of formula —CNS.

As used herein, the term “isothiocyanato” refers to a group of formula —NCS.

As used herein, the term “sulfinyl” refers to a group of formula —S(═O)—R.

As used herein, the term “S-sulfonamido” refers to a group of formula —S(═O)₂NR₂.

As used herein, the term “N-sulfonamido” refers to a group of formula RS(═O)₂NH—.

As used herein, the term “trihalomethanesulfonamido” refers to a group of formula X₃CS(═O)₂NR—.

As used herein, the term “O-carbamyl” refers to a group of formula —OC(═O)—NR₂.

As used herein, the term “N-carbamyl” refers to a group of formula ROC(═O)NH—.

As used herein, the term “O-thiocarbamyl” refers to a group of formula —OC(═S)—NR₂.

As used herein, the term “N-thiocarbamyl” refers to a group of formula ROC(═S)NH—.

As used herein, the term “C-amido” refers to a group of formula —C(═O)—NR₂.

As used herein, the term “N-amido” refers to a group of formula RC(═O)NH—.

As used herein, the term “ester” refers to a chemical moiety with formula —(R)ₙ—COOR′, where R and R′ are independently selected from among alkyl, cycloalkyl, aryl, heteroaryl (bonded through a ring carbon) and non-aromatic heterocycle (bonded through a ring carbon), where n is 0 or 1.

As used herein, the term “amide” refers to a chemical moiety with formula —(R)ₙ—C(O)NHR′ or —(R)ₙ—NHC(O)R′, where R and R′ are independently selected from among alkyl, cycloalkyl, aryl, heteroaryl (bonded through a ring carbon) and heteroalicyclic (bonded through a ring carbon), where n is 0 or 1. In certain embodiments, an amide can be an amino acid or a peptide.

As used herein, the terms “amine,” “hydroxy,” and “carboxyl” include such groups that have been esterified or amidified. Procedures and specific groups used to achieve esterification and amidification are known to those of skill in the art and can readily be found in reference sources such as Greene and Wuts, Protective Groups in Organic Synthesis, 3^{rd} Ed., John Wiley & Sons, New York, N.Y., 1999, which is incorporated herein in its entirety.

As used herein, the term “linked to form a ring” refers to instances where two atoms that are bound either to a single atom or to atoms that are themselves ultimately bound, are each bound to a linking group, such that the resulting structure forms a ring. That resulting ring contains the two atoms that are linked to form a ring, the atom (or atoms) that previously linked those atoms, and the linker. For example, if A and B below are “linked to form a ring”

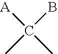

the resulting ring includes A, B, C, and a linking group. Unless otherwise indicated, that linking group can be of any length and can be optionally substituted. Referring to the above example, resulting structures include, but are not limited to:

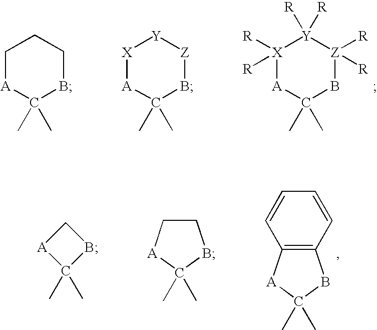

and the like.

In certain embodiments, the two substituents that together form a ring are not immediately bound to the same atom. For example, if A and B, below, are linked to form a ring:

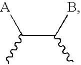

the resulting ring contains A, B, the two atoms that already link A and B and a linking group. Examples of resulting structures include, but are not limited to:

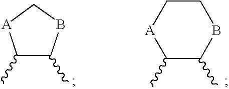

and the like.

In certain embodiments, the atoms that are linked to form a ring are separated by three or more atoms. For example, if A and B, below, are linked to form a ring:

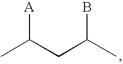

the resulting ring contains A, B, the 3 atoms that already link A and B, and a linking group. Examples of resulting structures include, but are not limited to:

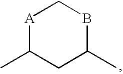

and the like.

Unless otherwise indicated, the term “optionally substituted,” refers to a group in which none, one, or more than one of the hydrogen atoms has been replaced with one or more group(s) individually and independently selected from among cycloalkyl, aryl, heteroaryl, non-aromatic heterocycle, hydroxy, alkoxy, aryloxy, mercapto, alkylthio, arylthio, cyano, halo, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalo-methanesulfonyl, and amino, including mono- and di-substituted amino groups, and the protected derivatives of amino groups. Such protective derivatives (and protecting groups that can form such protective derivatives) are known to those of skill in the art and can be found in references such as Greene and Wuts, above. In embodiments in which two or more hydrogen atoms have been substituted, the substituent groups can together form a ring.

Throughout the specification, groups and substituents thereof can be chosen by one skilled in the field to provide stable moieties and compounds.

As used herein, the term “carrier” refers to a compound that facilitates the incorporation of another compound into cells or tissues. For example, dimethyl sulfoxide (DMSO) is a commonly used carrier for improving incorporation of certain organic compounds into cells or tissues.

As used herein, the term “pharmaceutical composition” refers to a chemical compound or composition capable of inducing a desired therapeutic effect in a patient. In certain embodiments, a pharmaceutical composition contains an active agent, which is the agent that induces the desired therapeutic effect. In certain embodiments, a pharmaceutical composition contains a prodrug. In certain embodiments, a pharmaceutical composition contains inactive ingredients such as carriers, excipients, and the like.

As used herein, the term “therapeutically effective amount” refers to an amount of a pharmaceutical composition sufficient to achieve a desired therapeutic effect.

As used herein, a “prodrug” refers to a compound that is converted from a less active form into a corresponding more active form in vivo. In certain embodiments, upon in vivo administration, a prodrug is chemically converted to the biologically, pharmaceutically or therapeutically more active form of the compound. In certain embodiments, a prodrug is enzymatically metabolized by one or more steps or processes to the biologically, pharmaceutically or therapeutically active form of the compound. To produce a prodrug, a pharmaceutically active compound is modified such that the active compound will be regenerated upon in vivo administration. The prodrug can be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

As used herein, the term “pharmaceutically acceptable” refers to a formulation of a compound that does not significantly abrogate the biological activity, a pharmacological activity and/or other properties of the compound when the formulated compound is administered to a patient. In certain embodiments, a pharmaceutically acceptable formulation does not cause significant irritation to a patient.

As used herein, “pharmaceutically acceptable derivatives” of a compound include, but are not limited to, salts, esters, enol ethers, enol esters, acetals, ketals, orthoesters, hemiacetals, hemiketals, acids, bases, solvates, hydrates or prodrugs thereof. Such derivatives can be readily prepared by those of skill in this art using known methods for such derivatization. The compounds produced can be administered to animals or humans without substantial toxic effects and either are pharmaceutically active or are prodrugs. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethylbenzimidazole, diethyl amine and other alkylamines, piperazine and tris(hydroxymethyl)amino-methane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, salts of mineral acids, such as but not limited to hydro-chlorides and sulfates; and salts of organic acids, such as but not limited to acetates, lactates, malates, tartrates, citrates, ascorbates, succinates, butyrates, valerates and fumarates. Pharmaceutically acceptable esters include, but are not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is hydrogen, alkyl, alkenyl, alkynyl, aryl, heteroaryl, aralkyl, heteroaralkyl, cycloalkyl or heterocyclyl. Pharmaceutically acceptable enol esters include, but are not limited to, derivatives of formula C═C(OC(O)R) where R is hydrogen, alkyl, alkenyl, alkynyl, aryl, heteroaryl, aralkyl, heteroaralkyl, cycloalkyl or heterocyclyl. Pharmaceutically acceptable solvates and hydrates are complexes of a compound with one or more solvent or water molecules, or 1 to about 100, or 1 to about 10, or one to about 2, 3 or 4, solvent or water molecules.

It is to be understood that the compounds provided herein can contain chiral centers. Such chiral centers can be of either the (R) or (S) configuration, or can be a mixture thereof. Thus, the compounds provided herein can be enantiomerically pure, or be stereoisomeric or diastereomeric mixtures.

As used herein, the term “substantially pure” means sufficiently homogeneous to appear free of readily detectable impurities as determined by standard methods of analysis, such as thin layer chromatography (TLC), gel electrophoresis, high performance liquid chromatography (HPLC) and mass spectrometry (MS), used by those of skill in the art to assess such purity, or sufficiently pure such that further purification would not detectably alter the physical and chemical properties, such as enzymatic and biological activities, of the substance. Thus, substantially pure object species (e.g., compound) is the predominant species present (i.e., on a molar basis it is more abundant than any other individual species in the composition). In certain embodiments, a substantially purified fraction is a composition wherein the object species contains at least about 50 percent (on a molar basis) of all species present. In certain embodiments, a substantially pure composition will contain more than about 50%, 60%, 70%, 80%, 85%, 90%, 95%, or 99% of all species present in the composition. In certain embodiments, a substantially pure composition will contain more than about 80%, 85%, 90%, 95%, or 99% of all species present in the composition. Methods for purification of the compounds to produce substantially chemically pure compounds are known to those of skill in the art. A substantially chemically pure compound can, however, be a mixture of stereoisomers. In such instances, further purification might increase the specific activity of the compound. The instant disclosure is meant to include all such possible isomers, as well as their racemic and optically pure forms. Optically active (+) and (−), (R)- and (S)-, or (D)- and (L)-isomers can be prepared using chiral synthons or chiral reagents, or resolved using conventional techniques, such as reverse phase HPLC. When the compounds described herein contain olefinic double bonds or other centers of geometric asymmetry, and unless specified otherwise, it is intended that the compounds include E and Z geometric isomers. Likewise, all tautomeric forms also are included.

As used herein, the term “co-administer” refers to administering more than one pharmaceutical agent to a patient. In certain embodiments, co-administered pharmaceutical agents are administered together in a single dosage unit. In certain embodiments, co-administered pharmaceutical agents are administered separately. In certain embodiments, co-administered pharmaceutical agents are administered at the same time. In certain embodiments, co-administered pharmaceutical agents are administered at different times.

As used herein, the term “subject” is an animal, typically a mammal, including human.

As used herein, the term “patient” includes human and animal subjects.

As used herein, the term “tissue-selective” refers to the ability of a compound to modulate a biological activity in one tissue to a greater or lesser degree than it modulates a biological activity in another tissue. The biological activities in the different tissues can be the same or they can be different. The biological activities in the different tissues can be mediated by the same type of target receptor. For example, in certain embodiments, a tissue-selective compound can modulate an androgen receptor mediated biological activity in one tissue and fail to modulate, or modulate to a lesser degree, an androgen receptor mediated biological activity in another tissue type.

As used herein, the term “monitoring” refers to observing an effect or absence of any effect. In certain embodiments, one monitors cells after contacting those cells with a compound provided herein. Examples of effects that can be monitored include, but are not limited to, changes in cell phenotype, cell proliferation, androgen receptor activity, or the interaction between an androgen receptor and a natural binding partner.

As used herein, “a condition susceptible to treatment with an androgen receptor modulator” refers to condition that responds to treatment with an androgen receptor modulator, such as an androgen receptor agonist or androgen receptor antagonist, or where one or more symptoms of the condition, disease or disorder is ameliorated by treatment by an androgen receptor modulator, or where the condition, disease or disorder is affected by androgen receptor activity, or in which androgen receptor activity is implicated, or in which androgen receptor activity is an underlying etiology. The conditions are respond to androgen agonist or antagonist therapy. Examples include, but are not limited to benign prostatic hyperplasia, acne, seborrhea, hirsutism, androgenic alopecia, hair loss, and hyperandrogenism, male-pattern baldness, wasting diseases, hypogonadism, infertility, impotence, frailty or age-related functional decline in the elderly; catabolic side effects of glucocorticoids; osteoporosis; osteopenia; chronic fatigue syndrome; chronic myalgia; acute fatigue syndrome and muscle loss; wound healing; bone fracture repair; post-surgical adhesions; periodontal disease; cardiomyopathy; thrombocytopenia; growth retardation in connection with Crohn's disease; short bowel syndrome; irritable bowel syndrome; inflammatory bowel disease; Crohn's disease; ulcerative colitis; complications associated with transplantation; physiological short stature associated with growth hormone deficiency; short stature associated with chronic illness; obesity; growth retardation associated with obesity; anorexia; hypercortisolism; Cushing's syndrome; Paget's disease; Alzheimer's disease; osteoarthritis; pulsatile growth hormone release; osteochondrodysplasias; depression, nervousness, irritability or stress; reduced mental energy; low self-esteem; catabolism in connection with pulmonary dysfunction and ventilator dependency; cardiac dysfunction; elevated blood pressure; ventricular dysfunction; reperfusion events; chronic dialysis; protein catabolic responses following trauma; cachexia and protein loss due to chronic illness; hyperinsulinemia; nesidioblastosis; wasting in connection with multiple sclerosis or other neurodegenerative disorders; metabolic homeostasis and renal homeostasis; insulin resistance; insulin resistance in the heart; hypothermia; congestive heart failure; lipodystrophy; muscular atrophy; musculoskeletal impairment; sleep disorders; catabolic state of prolonged critical illness; seborrhea; anemia; hyperpilosity; benign prostate hypertrophy; adenomas and neoplasms of the prostate; malignant tumor cells containing the androgen receptor; cancers of the skin, pancreas, endometrium, lung, colon, breast, brain, ovaries, bladder, lymphatic, liver and kidney; osteosarcoma; hypercalcemia of malignancy; metastatic bone disease; spermatogenesis; endometriosis; polycystic ovary syndrome; preeclampsia; eclampsia of pregnancy; preterm labor; premenstrual syndrome; vaginal dryness; age related decreased testosterone levels in men; male menopause; hypogonadism; male and female sexual dysfunction; and Reaven's Syndrome. See Singh et ah, Current Medicinal Chemistry (2000) 7: 211-247; Boyer, Australian Prescriber (1996) 19: 22-24; and Claman et al., J Obstet Gynaecol Can (January 2002) 24(1): 62-67).

As used herein, the term “cell phenotype” refers to physical or biological characteristics. Examples of characteristics that constitute phenotype include, but are not limited to, cell size, cell proliferation, cell differentiation, cell survival, apoptosis (cell death), or the utilization of a metabolic nutrient (e.g., glucose uptake). Certain changes or the absence of changes in cell phenotype are readily monitored using techniques known in the art.

As used herein, “a disease or disorder associated with androgen receptor activity” refers to disease or disorder in which androgen receptor activity is implicated or in which androgen receptor activity is an underlying etiology. Such diseases respond to treatment with an androgen receptor modulator. Examples of such diseases or disorders include, but are not limited to, frailty or age-related functional decline in the elderly; catabolic side effects of glucocorticoids; osteoporosis; osteopenia; chronic fatigue syndrome; chronic myalgia; acute fatigue syndrome and muscle loss; wound healing; bone fracture repair; post-surgical adhesions; periodontal disease; wasting secondary to fractures; wasting in connection with chronic obstructive pulmonary disease; wasting in connection with chronic liver disease; wasting in connection with AIDS, cancer cachexia, burn and trauma recovery, chronic catabolic state, eating disorders and chemotherapy; cardiomyopathy; thrombocytopenia; growth retardation in connection with Crohn's disease; short bowel syndrome; irritable bowel syndrome; inflammatory bowel disease; Crohn's disease; ulcerative colitis; complications associated with transplantation; physiological short stature associated with growth hormone deficiency; short stature associated with chronic illness; obesity; growth retardation associated with obesity; anorexia; hypercortisolism; Cushing's syndrome; Paget's disease; Alzheimer's disease; osteoarthritis; pulsatile growth hormone release; osteochondrodysplasias; depression, nervousness, irritability or stress; reduced mental energy; low self-esteem; catabolism in connection with pulmonary dysfunction and ventilator dependency; cardiac dysfunction; elevated blood pressure; ventricular dysfunction; reperfusion events; chronic dialysis; protein catabolic responses following trauma; cachexia and protein loss due to chronic illness; hyperinsulinemia; nesidioblastosis; wasting in connection with multiple sclerosis or other neurodegenerative disorders; metabolic homeostasis and renal homeostasis; insulin resistance; insulin resistance in the heart; hypothermia; congestive heart failure; lipodystrophy; muscular atrophy; musculoskeletal impairment; sleep disorders; catabolic state of prolonged critical illness; hirsutism; acne; seborrhea; androgenic alopecia; anemia; hyperpilosity; benign prostate hypertrophy; adenomas and neoplasms of the prostate; malignant tumor cells containing the androgen receptor; cancers of the skin, pancreas, endometrium, lung, colon, breast, brain, ovaries, bladder, lymphatic, liver and kidney; osteosarcoma; hypercalcemia of malignancy; metastatic bone disease; spermatogenesis; endometriosis; polycystic ovary syndrome; preeclampsia; eclampsia of pregnancy; preterm labor; premenstrual syndrome; vaginal dryness; age related decreased testosterone levels in men; male menopause; hypogonadism; male and female sexual dysfunction; hair loss; and Reaven's Syndrome.

As used herein, the term “contacting” refers to bringing two or more materials into close enough proximity that they can interact. In certain embodiments, contacting can be accomplished in a vessel such as a test tube, a petri dish, or the like. In certain embodiments, contacting can be performed in the presence of additional materials. In certain embodiments, contacting can be performed in the presence of cells. In certain of such embodiments, one or more of the materials that are being contacted can be inside a cell. Cells can be alive or can be dead. Cells can or can not be intact.

### B. Compounds

Certain compounds that bind to androgen receptors and/or modulate an activity of such receptors play a role in health (e.g., normal growth, development, and/or absence of disease). In certain embodiments, selective androgen receptor modulators, binding compounds, and/or degrading compounds are useful for treating any of a variety of diseases or conditions.

Certain compounds have been previously described as receptor modulators or as possible receptor modulators, receptor reducing compounds and/or receptor degrading compounds. See e.g., U.S. Pat. Nos. 6,462,038, 5,693,646; 6,380,207; 6,506,766; 5,688,810; 5,696,133; 6,569,896, 6,673,799; 4,636,505; 4,097,578; 3,847,988; 6,861,532; 4,659,516; 6,566,372; 6,696,459; 6,017,924; U.S. application Ser. Nos. 10/209,461 (Pub. No. US 2003/0055094), 10/758,582 (Pub No. US 2005/0085467), 10/493,013 (Pub. No. US 2004/0198717 A1); 10/080,926 (Pub No. US 2002/0183346); 10/080,503 (Pub No. US 2002/0183314); WO 01/27086; WO 02/22585; WO 2005/000795 A2; WO 2005/037206 A2; WO 05/018573; PCT US05/007867; Zhi, et al. Bioorganic & Medicinal Chemistry Letters 2000, 10, 415-418; Pooley, et. al., J. Med. Chem. 1998, 41, 3461; Hamann, et al. J. Med. Chem. 1998, 41(4), 623; and Yin, et al., Molecular Pharmacology, 2003, 63 (1), 211-223, the disclosures of which are incorporated in their entirety.

In certain embodiments, the compounds provided herein are selective androgen receptor modulators. In certain embodiments, the compounds provided herein are selective androgen receptor binding compounds. In certain embodiments, the compounds provided herein are androgen receptor reducing compounds. In certain embodiments, the compounds provided herein are selective androgen receptor degrading compounds.

In certain embodiments, provided herein are methods of making and methods of using androgen receptor modulators, androgen binding compounds, and or selective androgen receptor reducing compounds provided herein. In certain embodiments, selective androgen modulators are agonists, partial agonists, and/or antagonists for the androgen receptor.

In certain embodiments, the compounds provided herein have a structure selected from among Formula I or Formula II:

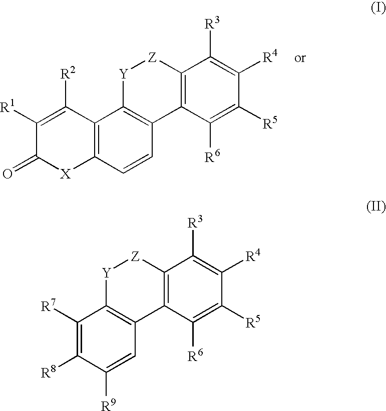

and pharmaceutically acceptable salts and prodrugs thereof.

In certain embodiments, R¹ is selected from among hydrogen, F, Cl, C₁-C₄ alkyl, and C₁-C₄ haloalkyl. In certain embodiments, R¹ is selected from among hydrogen, F, Cl, an optionally substituted C₁-C₄ alkyl, and an optionally substituted C₁-C₄ haloalkyl. In certain embodiments, R¹ is selected from among hydrogen, F, Cl, a fully saturated C₁-C₄ alkyl, and a fully saturated C₁-C₄ haloalkyl.

In certain embodiments, R¹ is selected from among hydrogen, F, Cl and C₁-C₄ alkyl. In certain embodiments, R¹ is selected from among hydrogen, F, and CI. In certain embodiments, R¹ is selected from among hydrogen and C₁-C₄ alkyl. In certain embodiments, R¹ is hydrogen.

In certain embodiments, R² is selected from among hydrogen, halogen, C₁-C₄ alkyl, C₁-C₆ haloalkyl, C₁-C₆ heteroalkyl, and C₁-C₄ haloheteroalkyl. In certain embodiments, R² is selected from among hydrogen, halogen, an optionally substituted C₁-C₆ alkyl, an optionally substituted C₁-C₆ haloalkyl, an optionally substituted C₁-C₆ heteroalkyl, and an optionally substituted C₁-C₄ haloheteroalkyl.

In certain embodiments, R² is selected from among hydrogen, halogen, C₁-C₆ alkyl and C₁-C₆ haloalkyl. In certain embodiments, R² is selected from among halogen, C₁-C₆ alkyl, C₁-C₆ haloalkyl C₁-C₆ heteroalkyl, and C₁-C₄ haloheteroalkyl. In certain embodiments, R² is selected from among halogen, C₁-C₆ alkyl and C₁-C₆ haloalkyl. In certain embodiments, R² is selected from among C₁-C₆ alkyl and C₁-C₆ haloalkyl. In certain embodiments, R² is selected from among methyl and trifluoromethyl. In certain embodiments, R² is methyl. In certain embodiments, R² is trifluoromethyl.

In certain embodiments, R³ and R⁴ are each independently selected from among hydrogen, halogen, C₁-C₆ alkyl, C₁-C₆ haloalkyl, C₁-C₆ heteroalkyl, and C₁-C₆ haloheteroalkyl. In certain embodiments, R³ and R⁴ are each independently selected from among hydrogen, halogen, an optionally substituted C₁-C₆ alkyl, an optionally substituted C₁-C₆ haloalkyl, an optionally substituted C₁-C₆ heteroalkyl, and an optionally substituted C₁-C₆ haloheteroalkyl.

In certain embodiments, R³ and R⁴ are each independently selected from among hydrogen, halogen, C₁-C₆ alkyl and C₁-C₆ haloalkyl. In certain embodiments, R³ and R⁴ are each independently selected from among hydrogen, halogen and C₁-C₆ alkyl. In certain embodiments, R³ and R⁴ are each independently hydrogen.

In certain embodiments, R⁵ and R⁶ are each independently selected from among hydrogen, halogen, OR^{A}, S(O)ₙR^{A}, NR^{A}R^{B}, NR^{A}S(O)ₙR^{B}, COR^{A}, CO₂R^{A}, OC(O)R^{A}, CH₂OR^{A}, CONR^{A}R^{B}, an optionally substituted C₁-C₆ alkyl, an optionally substituted C₁-C₆ haloalkyl, and an optionally substituted C₁-C₆ heteroalkyl. In certain embodiments, R⁵ and R⁶ are linked to form a heterocyclic ring.

In certain embodiments, R⁵ and R⁶ are each independently selected from among hydrogen, halogen, OR^{A}, COR^{A}, CO₂R^{A}, OC(O)R^{A}, CH₂OR^{A}, C₁-C₆ alkyl, C₁-C₆ haloalkyl, and C₁-C₆ heteroalkyl. In certain embodiments, R⁵ and R⁶ are each independently selected from among hydrogen, halogen, OR^{A}, COR^{A}, CO₂R^{A}, OC(O)R^{A}, and CH₂OR^{A}. In certain embodiments, R⁵ and R⁶ are each independently selected from among hydrogen, halogen, OR^{A}, OC(O)R^{A}, and CH₂OR^{A}. In certain embodiments, R⁵ and R⁶ are each independently selected from among hydrogen, OR^{A} and OC(O)R^{A}.

In certain embodiments, R⁵ and R⁶ are each independently selected from among hydrogen, F, hydroxy, methoxy, ethoxy, isopropoxy, hydroxymethyl, OC(O)^{t}Bu and CO₂Me. In certain embodiments, R⁵ and R⁶ are each independently selected from among hydrogen, hydroxy, methoxy and OC(O)^{t}Bu.

In certain embodiments, R⁷ and R⁹ are each independently selected from among hydrogen, halogen, OR^{A}, C₁-C₆ alkyl, C₁-C₆ haloalkyl, C₁-C₆ heteroalkyl, and C₁-C₆ haloheteroalkyl.

In certain embodiments, R⁷ and R⁹ are each independently selected from among hydrogen, halogen and C₁-C₆ alkyl. In certain embodiments, R⁷ and R⁹ are each independently selected from among hydrogen and C₁-C₆ alkyl. In certain embodiments, R⁷ and R⁹ are each independently selected from among hydrogen and methyl. In certain embodiments, R⁷ is methyl and R⁹ is hydrogen. In certain embodiments, R⁷ is methyl.

In certain embodiments, R⁷ is selected from among halogen, OR^{A}, C₁-C₆ alkyl, C₁-C₆ haloalkyl, C₁-C₆ heteroalkyl, and C₁-C₆ haloheteroalkyl. In certain embodiments, R⁷ is selected from among halogen, C₁-C₆ alkyl and C₁-C₆ haloalkyl. In certain embodiments, R⁷ is selected from among halogen and C₁-C₆ alkyl. In certain embodiments, R⁷ is C₁-C₆ alkyl. In certain embodiments, R⁹ is selected from among hydrogen, halogen, OR^{A}, C₁-C₆ alkyl, C₁-C₆ haloalkyl, C₁-C₆ heteroalkyl, and C₁-C₆ haloheteroalkyl. In certain embodiments, R⁹ is selected from among hydrogen, halogen, C₁-C₆ alkyl, and C₁-C₆ haloalkyl. In certain embodiments, R⁹ is selected from among hydrogen, halogen, and C₁-C₆ alkyl. In certain embodiments, R⁹ is selected from among hydrogen, halogen, and C₁-C₆ alkyl.

In certain embodiments, R⁸ is selected from among NO₂, CN, COR^{A}, CO₂R^{A}, CONR^{A}R^{B}, SOR^{A}, SO₂R^{A}, and SO₂NR^{A}R^{B}, C₁-C₆ haloalkyl, and C₁-C₄ haloheteroalkyl. In certain embodiments, R⁸ is selected from among NO₂, CN, COR^{A}, CO₂R^{A}, CONR^{A}R^{B}, SOR^{A}, SO₂R^{A}, and SO₂NR^{A}R^{B}. In certain embodiments, R⁸ is selected from among NO₂, C₁-C₆ haloalkyl, and C₁-C₄ haloheteroalkyl. In certain embodiments, R⁸ is selected from among NO₂, SOR^{A}, SO₂R^{A} and SO₂NR^{A}R^{B}. In certain embodiments, R⁸ is NO₂.

In certain embodiments, R^{A} and R^{B} are each independently selected from among hydrogen, C₁-C₆ alkyl, C₁-C₆ haloalkyl, and C₁-C₆ heteroalkyl. In certain embodiments, R^{A} and R^{B} are linked to form a ring. In certain embodiments, R^{A} and R^{B} are each independently selected from among hydrogen and C₁-C₆ alkyl. In certain embodiments, R^{A} and R^{B} are each independently selected from among hydrogen and methyl.

In certain embodiments, X is selected from among O, S, and NR^{A}. In certain embodiments, X is selected from among O and NR^{A}. In certain embodiments, X is NR^{A}.

In certain embodiments, Y and Z are each independently selected from among CR^{A}R^{B}, CR^{A}OR^{B}, CO, OCH₂, CH₂O, O, S(O)ₙ, and NR^{A}. In certain embodiments, Y and Z are each independently selected from among CR^{A}R^{B}, CR^{A}OR^{B}, CO, OCH₂, CH₂O and O. In certain embodiments, Y and Z are each independently selected from among CR^{A}R^{B}, CR^{A}OR^{B}, CO and O. In certain embodiments, Y and Z are each independently selected from among CR^{A}OR^{B}, CO and O. In certain embodiments, Y and Z are not the same. In certain embodiments, Y and Z are the same.

In certain embodiments, n is selected from among 0, 1, and 2.

In certain embodiments, if X is NH, and Y is CO, and Z is O, and each of R¹, R³, R⁴, and R⁵ is hydrogen, and R² is CH₃, then R⁶ is not OCH₃.

In certain embodiments, if Y is CO, and Z is O, and R⁸ is NO₂, and R⁹ is hydrogen, then R⁷ is not hydrogen.

In certain embodiments, if R⁸ is NO₂, CN, COR^{A}, CO₂R^{A} or CONR^{A}R^{B}, and Y or Z is CO, and R⁹ is hydrogen, then R⁷ is not hydrogen. In certain embodiments, if R⁸ is CN, CO₂R^{A} or COR^{A}, and R⁷ is methyl, then one of R⁵, R⁶ or R⁹ is not hydrogen.

In certain embodiments, if R⁸ is NO₂, CN, COR^{A}, CO₂R^{A} or CONR^{A}R^{B}, and Y or Z is CO, and R⁹ is hydrogen, then R⁷ is not hydrogen; or if R⁸ is CN, CO₂R^{A} or COR^{A}, and R⁷ is methyl, then one of R⁵, R⁶ or R⁹ is not hydrogen; or Y and Z are not the same.

In certain embodiments, if R⁸ is NO₂, CN, COR^{A}, CO₂R^{A} or CONR^{A}R^{B}, and R⁹ is hydrogen, then R⁷ is not hydrogen. In certain embodiment, if R⁸ is COR^{A} or CO₂R^{A}, and R⁹ is methoxy, then R⁷ is not hydrogen. In certain embodiments, if R⁸ is CN or COR^{A}, and R⁷ is methyl, then R⁹ is not hydrogen. In certain embodiments, if R⁸ is NO₂, CN, COR^{A}, CO₂R^{A} or CONR^{A}R^{B}, and R⁹ is hydrogen, then R⁷ is not hydrogen; or if R⁸ is COR^{A} or CO₂R^{A}, and R⁹ is methoxy, then R⁷ is not hydrogen; or if R⁸ is CN or COR^{A}, and R⁷ is methyl, then R⁹ is not hydrogen. In certain embodiments, if R⁸ is NO₂, CN, COR^{A}, CO₂R^{A} or CONR^{A}R^{B}, and R⁹ is hydrogen, then R⁷ is not hydrogen; or if R⁸ is COR^{A} or CO₂R^{A}, and R⁹ is methoxy, then R⁷ is not hydrogen; or if R⁸ is CN or COR^{A}, and R⁷ is methyl, then R⁹ is not hydrogen; or if the compound has a structure of Formula II, then Y and Z are not the same. In certain embodiments, if R⁸ is NO₂, CN, COR^{A}, CO₂R^{A}, CONR^{A}R^{B}, SOR^{A}, SO₂R^{A}, SO₂NR^{A}R^{B}, C₁-C₆ haloalkyl, or C₁-C₄ haloheteroalkyl, then Y and Z are not the same.

In certain embodiments, if the compound has a structure of Formula II and if Y is the same as Z, then R⁴ is not isopropyl. In certain embodiments, if the compound has a structure of Formula II then at least one of R³, R⁴, R⁵, R⁶, and R⁹ is not hydrogen. In certain embodiments, if R⁸ is NO₂ and Y is CO and Z is O and R⁷ is hydrogen then R⁹ is not hydrogen. In certain embodiments, if R⁸ is CO₂R^{A} or CONR^{A}R^{B} then R⁴ is not F or R⁹ is not methoxy.

In certain embodiments, if the compound has a structure of Formula II and if Y is the same as Z, then R⁴ is not isopropyl; or if the compound has a structure of Formula II then at least one of R³, R⁴, R⁵, R⁶, and R⁹ is not hydrogen; or if R⁸ is NO₂ and Y is CO and Z is O and R⁷ is hydrogen then R⁹ is not hydrogen; or if R⁸ is CO₂R^{A} or CONR^{A}R^{B} then R⁴ is not F or R⁹ is not methoxy.

In embodiments in which two or more of a particular group are present, the identities of those two or more particular groups are selected independently and, thus, can be the same or different from one another. For example, certain compounds provided herein contain two or more R^{A} groups. The identities of those two or more R^{A} groups are each selected independently. Thus, in certain embodiments, those R^{A} groups are all the same as one another; in certain embodiments, those R^{A} groups are all different from one another; and in certain embodiments, some of those R^{A} groups are the same as one another and some are different from one another. This independent selection applies to any group that is present in a compound more than once.

Any combination of the groups described above for the various variables is contemplated herein.

In certain embodiments, a compound of Formula I or Formula II is a selective androgen receptor modulator. In certain embodiments, a compound of Formula I or Formula II is a selective androgen receptor agonist. In certain embodiments, a compound of Formula I or Formula II is a selective androgen receptor antagonist. In certain embodiments, a compound of Formula I or Formula II is a selective androgen receptor partial agonist. In certain embodiments, a compound of Formula I or Formula II is a tissue-specific selective androgen modulator. In certain embodiments, a compound of Formula I or Formula II is a selective androgen receptor binding compound. In certain embodiments, a compound of Formula I or Formula II is a selective androgen receptor reducing compound. In certain embodiments, a compound Formula I or Formula II is a selective androgen receptor degrading compound.

In certain embodiments, provided herein is a compound selected from among:
9-Fluoro-4-methyl-1H-6-oxa-1-aza-chrysene-2,5-dione (Compound 101);
2,2-Dimethyl-propionic acid 10-methoxy-4-methyl-2,5-dioxo-2,5-dihydro-1H-6-oxa-1-aza-chrysen-9-yl ester (Compound 102);
9-Methoxy-4-methyl-1H-6-oxa-1-aza-chrysene-2,5-dione (Compound 103);
2,2-Dimethyl-propionic acid 4-methyl-2,5-dioxo-2,5-dihydro-1H-6-oxa-1-aza-chrysen-10-yl ester (Compound 104);
5-Hydroxy-10-methoxy-4-methyl-1H,5H-6-oxa-1-aza-chrysen-2-one (Compound 105);
5,10-Dimethoxy-4-methyl-1H,5H-6-oxa-1-aza-chrysen-2-one (Compound 106);
(±)-10-Methoxy-4,5-dimethyl-1H,5H-6-oxa-1-aza-chrysen-2-one (Compound 107);
(±)-5,10-Dimethoxy-4,5-dimethyl-1H,5H-6-oxa-1-aza-chrysen-2-one (Compound 108);
10-Methoxy-4-methyl-1H,5H-6-oxa-1-aza-chrysen-2-one (Compound 109);
5-Allyl-10-methoxy-4-methyl-1H,5H-6-oxa-1-aza-chrysen-2-one (Compound 110);
4-Methyl-2,5-dioxo-2,5-dihydro-1H-6-oxa-1-aza-chrysene-10-carboxylic acid methyl ester (Compound 111);
10-Hydroxymethyl-4-methyl-1H,5H-6-oxa-1-aza-chrysen-2-one (Compound 112);
10-Hydroxymethyl-4-trifluoromethyl-1H,5H-6-oxa-1-aza-chrysen-2-one (Compound 113);
5-Hydroxy-10-methoxy-4-trifluoromethyl-1H,5H-6-oxa-1-aza-chrysen-2-one (Compound 114);
10-Hydroxy-4-trifluoromethyl-1H,5H-6-oxa-1-aza-chrysen-2-one (Compound 115);
2,2-Dimethyl-propionic acid 2-oxo-4-trifluoromethyl-2,5-dihydro-1H-6-oxa-1-aza-chrysen-10-yl ester (Compound 116);
9-Hydroxy-10-methoxy-4-methyl-1H-6-oxa-1-aza-chrysene-2,5-dione (Compound 117);
9-Hydroxy-5,10-dimethoxy-4-methyl-1H,5H-6-oxa-1-aza-chrysen-2-one (Compound 118);
9-Hydroxy-10-methoxy-4-methyl-1H,5H-6-oxa-1-aza-chrysen-2-one (Compound 119);
9-Isopropoxy-10-methoxy-4-methyl-1H-6-oxa-1-aza-chrysene-2,5-dione (Compound 120);
9-Ethoxy-10-methoxy-4-methyl-1H-6-oxa-1-aza-chrysene-2,5-dione (Compound 121);
9-Ethoxy-1-ethyl-10-methoxy-4-methyl-1H-6-oxa-1-aza-chrysene-2,5-dione (Compound 122);
4-Methoxy-10-methyl-7,13-dihydro-12-oxa-7-aza-benzo[3,4]cyclohepta[1,2-α]naphthalene-8,11-dione (Compound 123);
10-Hydroxymethyl-4-methyl-1H-6-oxa-1-aza-chrysene-2,5-dione (Compound 124);
10-Hydroxy-4-trifluoromethyl-1H-5-oxa-1-aza-chrysene-2,6-dione (Compound 125);
10-Methoxy-4-trifluoromethyl-1H-5-oxa-1-aza-chrysene-2,6-dione (Compound 126);
10-Methoxy-4-trifluoromethyl-1,6-dihydro-5-oxa-1-aza-chrysen-2-one (Compound 127);
4-Trifluoromethyl-1,6-dihydro-5-oxa-1-aza-chrysen-2-one (Compound 128);
1-Hydroxy-7-methyl-8-nitro-benzo[c]chromen-6-one (Compound 129); and
1-Methoxy-7-methyl-8-nitro-benzo[c]chromen-6-one (Compound 130);

and pharmaceutically acceptable salts and prodrugs thereof.

In certain embodiments, provided herein is a compound selected from among:
9-Fluoro-4-methyl-1H-6-oxa-1-aza-chrysene-2,5-dione (Compound 101);
2,2-Dimethyl-propionic acid 10-methoxy-4-methyl-2,5-dioxo-2,5-dihydro-1H-6-oxa-1-aza-chrysen-9-yl ester (Compound 102);
9-Methoxy-4-methyl-1H-6-oxa-1-aza-chrysene-2,5-dione (Compound 103);
2,2-Dimethyl-propionic acid 4-methyl-2,5-dioxo-2,5-dihydro-1H-6-oxa-1-aza-chrysen-10-yl ester (Compound 104);
5-Hydroxy-10-methoxy-4-methyl-1H,5H-6-oxa-1-aza-chrysen-2-one (Compound 105);
5,10-Dimethoxy-4-methyl-1H,5H-6-oxa-1-aza-chrysen-2-one (Compound 106);
(±)-10-Methoxy-4,5-dimethyl-1H,5H-6-oxa-1-aza-chrysen-2-one (Compound 107);
(±)-5,10-Dimethoxy-4,5-dimethyl-1H,5H-6-oxa-1-aza-chrysen-2-one (Compound 108);
10-Methoxy-4-methyl-1H,5H-6-oxa-1-aza-chrysen-2-one (Compound 109);
5-Allyl-10-methoxy-4-methyl-1H,5H-6-oxa-1-aza-chrysen-2-one (Compound 110);
4-Methyl-2,5-dioxo-2,5-dihydro-1H-6-oxa-1-aza-chrysene-10-carboxylic acid methyl ester (Compound 111);
10-Hydroxymethyl-4-methyl-1H,5H-6-oxa-1-aza-chrysen-2-one (Compound 112);
10-Hydroxymethyl-4-trifluoromethyl-1H,5H-6-oxa-1-aza-chrysen-2-one (Compound 113);
5-Hydroxy-10-methoxy-4-trifluoromethyl-1H,5H-6-oxa-1-aza-chrysen-2-one (Compound 114);
10-Hydroxy-4-trifluoromethyl-1H,5H-6-oxa-1-aza-chrysen-2-one (Compound 115);
2,2-Dimethyl-propionic acid 2-oxo-4-trifluoromethyl-2,5-dihydro-1H-6-oxa-1-aza-chrysen-10-yl ester (Compound 116);
9-Hydroxy-10-methoxy-4-methyl-1H-6-oxa-1-aza-chrysene-2,5-dione (Compound 117);
9-Hydroxy-5,10-dimethoxy-4-methyl-1H,5H-6-oxa-1-aza-chrysen-2-one (Compound 118);
9-Hydroxy-10-methoxy-4-methyl-1H,5H-6-oxa-1-aza-chrysen-2-one (Compound 119);
9-Isopropoxy-10-methoxy-4-methyl-1H-6-oxa-1-aza-chrysene-2,5-dione (Compound 120);
9-Ethoxy-10-methoxy-4-methyl-1H-6-oxa-1-aza-chrysene-2,5-dione (Compound 121);
9-Ethoxy-1-ethyl-10-methoxy-4-methyl-1H-6-oxa-1-aza-chrysene-2,5-dione (Compound 122);
4-Methoxy-10-methyl-7,13-dihydro-12-oxa-7-aza-benzo[3,4]cyclohepta[1,2-α]-naphthalene-8,11-dione (Compound 123);
10-Hydroxymethyl-4-methyl-1H-6-oxa-1-aza-chrysene-2,5-dione (Compound 124);
10-Hydroxy-4-trifluoromethyl-1H-5-oxa-1-aza-chrysene-2,6-dione (Compound 125);
10-Methoxy-4-trifluoromethyl-1H-5-oxa-1-aza-chrysene-2,6-dione (Compound 126);
10-Methoxy-4-trifluoromethyl-1,6-dihydro-5-oxa-1-aza-chrysen-2-one (Compound 127); and
4-Trifluoromethyl-1,6-dihydro-5-oxa-1-aza-chrysen-2-one (Compound 128).

and pharmaceutically acceptable salts and prodrugs thereof.

In certain embodiments, provided herein is a compound selected from among:
2,2-Dimethyl-propionic acid 10-methoxy-4-methyl-2,5-dioxo-2,5-dihydro-1H-6-oxa-1-aza-chrysen-9-yl ester (Compound 102);
2,2-Dimethyl-propionic acid 4-methyl-2,5-dioxo-2,5-dihydro-1H-6-oxa-1-aza-chrysen-10-yl ester (Compound 104);
10-Hydroxymethyl-4-methyl-1H,5H-6-oxa-1-aza-chrysen-2-one (Compound 112);
10-Hydroxymethyl-4-trifluoromethyl-1H,5H-6-oxa-1-aza-chrysen-2-one (Compound 113);
10-Hydroxy-4-trifluoromethyl-1H,5H-6-oxa-1-aza-chrysen-2-one (Compound 115);
2,2-Dimethyl-propionic acid 2-oxo-4-trifluoromethyl-2,5-dihydro-1H-6-oxa-1-aza-chrysen-10-yl ester (Compound 116);
9-Hydroxy-10-methoxy-4-methyl-1H-6-oxa-1-aza-chrysene-2,5-dione (Compound 117);
9-Hydroxy-10-methoxy-4-methyl-1H,5H-6-oxa-1-aza-chrysen-2-one (Compound 119);
9-Isopropoxy-10-methoxy-4-methyl-1H-6-oxa-1-aza-chrysene-2,5-dione (Compound 120);
10-Hydroxymethyl-4-methyl-1H-6-oxa-1-aza-chrysene-2,5-dione (Compound 124);
10-Hydroxy-4-trifluoromethyl-1H-5-oxa-1-aza-chrysene-2,6-dione (Compound 125);
10-Methoxy-4-trifluoromethyl-1H-5-oxa-1-aza-chrysene-2,6-dione (Compound 126);
10-Methoxy-4-trifluoromethyl-1,6-dihydro-5-oxa-1-aza-chrysen-2-one (Compound 127);
4-Trifluoromethyl-1,6-dihydro-5-oxa-1-aza-chrysen-2-one (Compound 128); and
1-Hydroxy-7-methyl-8-nitro-benzo[c]chromen-6-one (Compound 129);

and pharmaceutically acceptable salts and prodrugs thereof.

In certain embodiments, provided herein is a compound selected from among:
2,2-Dimethyl-propionic acid 4-methyl-2,5-dioxo-2,5-dihydro-1H-6-oxa-1-aza-chrysen-10-yl ester (Compound 104);
10-Hydroxy-4-trifluoromethyl-1H,5H-6-oxa-1-aza-chrysen-2-one (Compound 115);
2,2-Dimethyl-propionic acid 2-oxo-4-trifluoromethyl-2,5-dihydro-1H-6-oxa-1-aza-chrysen-10-yl ester (Compound 116);
9-Hydroxy-10-methoxy-4-methyl-1H-6-oxa-1-aza-chrysene-2,5-dione (Compound 117);
9-Hydroxy-10-methoxy-4-methyl-1H,5H-6-oxa-1-aza-chrysen-2-one (Compound 119);
10-Hydroxy-4-trifluoromethyl-1H-5-oxa-1-aza-chrysene-2,6-dione (Compound 125);
4-Trifluoromethyl-1,6-dihydro-5-oxa-1-aza-chrysen-2-one (Compound 128);
1-Hydroxy-7-methyl-8-nitro-benzo[c]chromen-6-one (Compound 129);
   and pharmaceutically acceptable salts and prodrugs thereof.

### C. Preparation of Compounds

In certain embodiments, compounds provided herein can be synthesized using the following synthesis schemes. In each scheme, the variables (e.g., X, Y, Z, and R groups) correspond to the same definitions as those recited above.

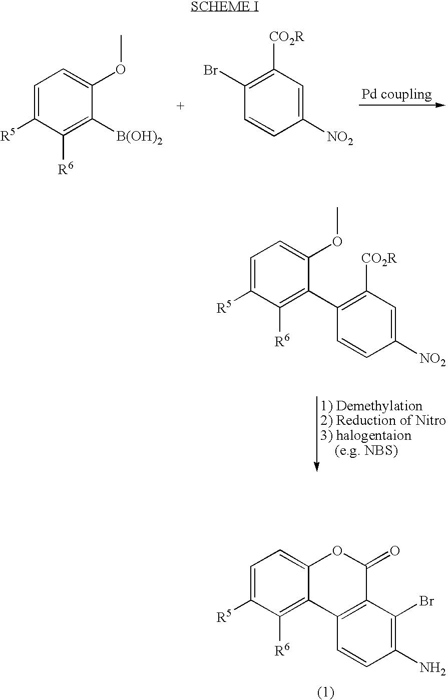

Scheme I describes the general synthesis of known benzocoumarin intermediates (1), described in U.S. Pat. No. 5,696,127, U.S. Pat. No. 6,001,846, U.S. Pat. No. 6,448,405, U.S. Pat. No. 6,506,766 and U.S. Pat. No. 6,358,947. Generally, treatment of 2-alkoxyphenylboronic acid, for example, 2-methoxyphenylboronic acid, with methyl 2-bromo-5-nitrobenzoate in the presence of a palladium catalyst, such as, for example, tetrakis(triphenyl-phosphine)palladium(0) or dichlorobis(triphenylphosphine)palladium(II), provides the biaryl. Demethylation, e.g., treatment with BBr₃, provides the lactone. Reduction of the nitro group to an amino with, for example, hydrogen over a metal catalyst, such as palladium on carbon, is followed by bromination, for example, with N-bromosuccinimide (NBS), to afford compound (1).

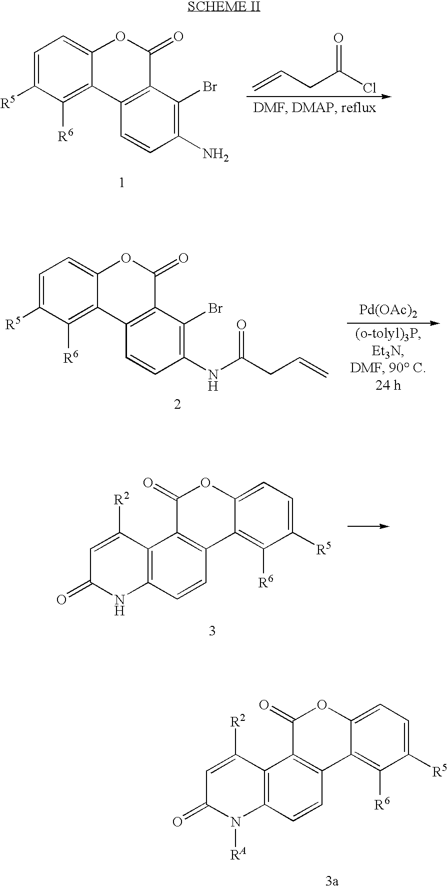

Scheme II describes synthesis of the tetracyclic quinolinones (3) from known benzocoumarin intermediates (1) described in U.S. Pat. No. 5,696,127, U.S. Pat. No. 6,001,846, U.S. Pat. No. 6,448,405, U.S. Pat. No. 6,506,766 and U.S. Pat. No. 6,358,947. Treatment of an aminobromo-benzocoumarin of structure 1 with 3-butenoyl chloride provides structure 2. Intramolecular palladium catalyzed cyclization provides structure 3.

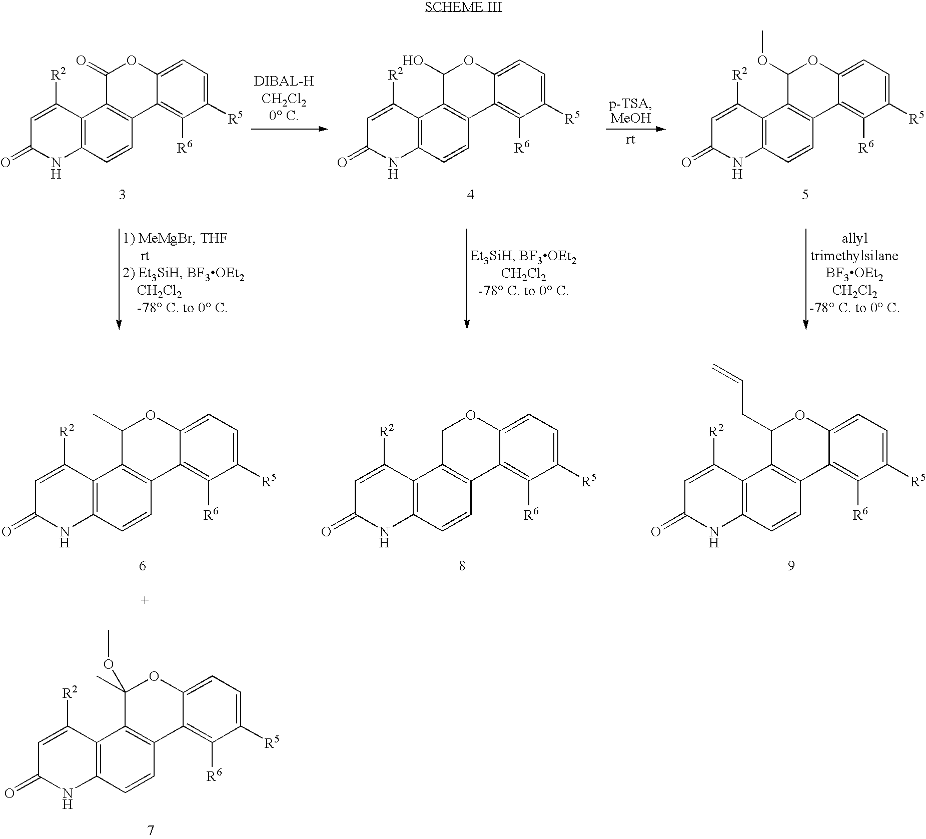

Scheme III describes the synthesis of a series of tetracyclic quinolinones from compounds of structure 3. Structures 6 and 7 can be prepared from structure 3 by Grignard reaction, followed by triethylsilane reduction in the presence of boron trifluoride etherate. Reduction of structure 3 by diisobutylaluminum hydride provides structure 4. Treatment of structure 4 with triethylsilane and boron trifluoride etherate provides structure 8. Treatment of structure 4 with acid (such as p-TSA) in methanol provides structure 5, Structure 9 can be prepared by treating structure 5 with allyltrimethylsilane in the presence of boron trifluoride etherate.

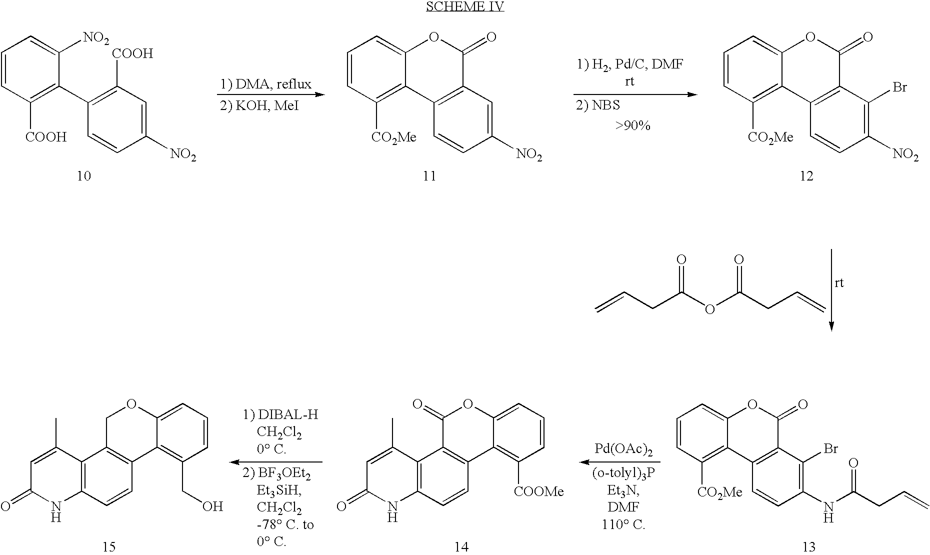

Scheme IV describes the preparation of compound 15. Refluxing compound 10 in dimethylacetamide followed by methylation with methyl iodide affords structure 11. Reduction and bromination of structure 11 affords structure 12, which undergoes acylation to afford structure 13. Intramolecular palladium catalyzed cyclization of structure 13 affords structure 14. Treatment of structure 14 with diisobutylaluminum hydride followed by triethylsilane in the presence of boron trifluoride affords structure 15.

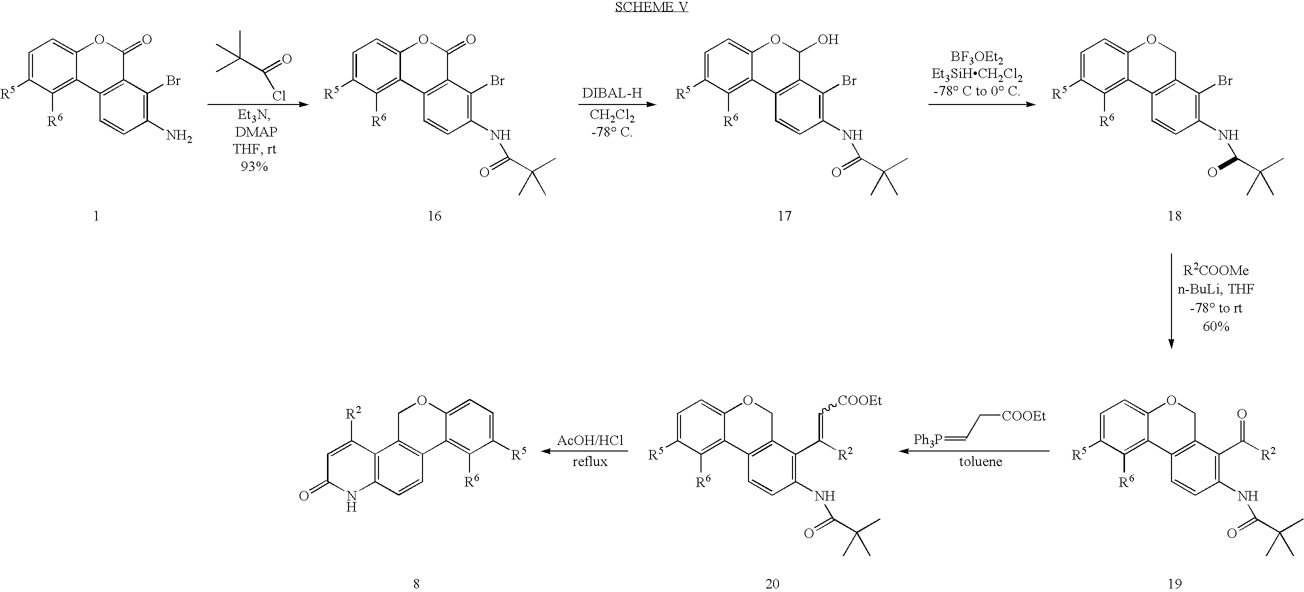

Scheme V describes an alternate synthesis of the tetracyclic quinolinones of structure 8 from a known benzocoumarin intermediates 1. Acylation of structure 1 affords structure 16. Reduction of structure 16 by diisobutylaluminum affords structure 17, which can be treated with triethylamine in the presence of boron trifluoride to afford structure 18. Treatment of structure 18 with n-butyllithium followed by a methyl ester affords structure 19, Structure 20 can be prepared by Wittig reaction from structure 19. Treatment of structure 20 by refluxing in acid affords structure 8.

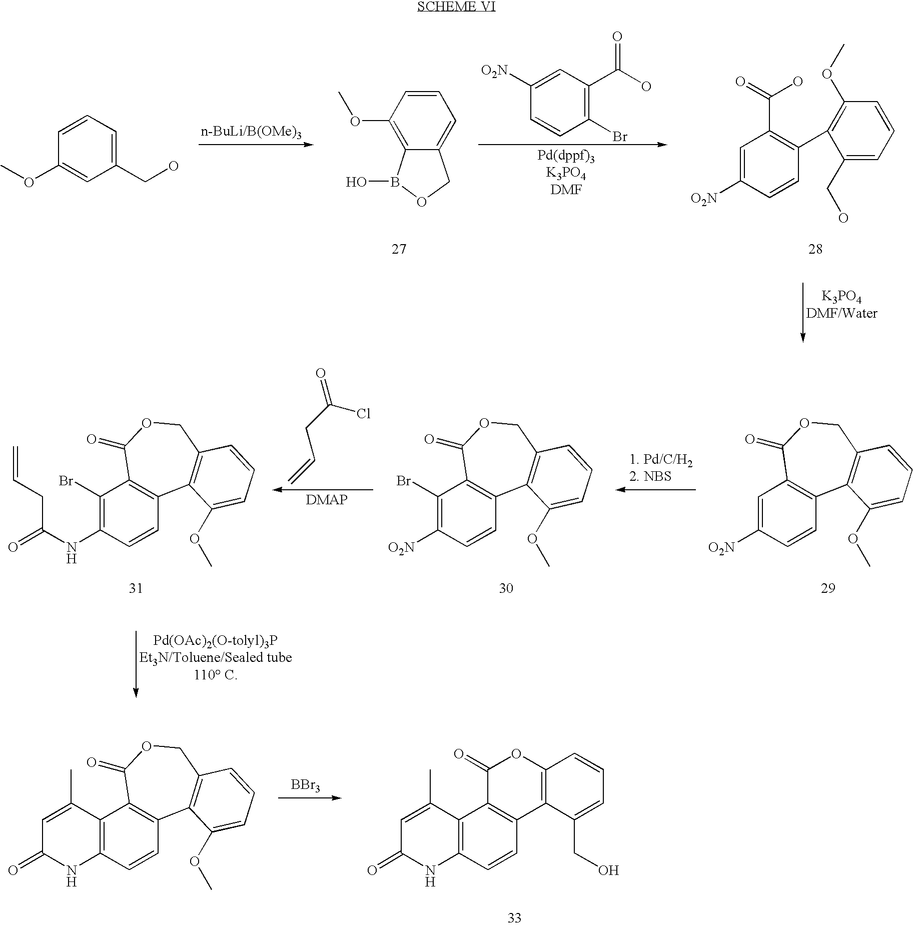

Scheme VI describes the synthesis of compounds 32 and 33. Treatment of commercially available 3-methoxybenzyl alcohol with n-butyllithium and trimethyl borate affords structure 27, Structure 28 can be prepared by palladium coupling reaction of structure 27 and commercially available 2-bromo-5-nitrobenzoic acid. Treatment of structure 28 with K₃PO₄ in DMF and water affords structure 29. Reduction of the nitro group followed by bromination affords compound 30, which undergoes acylation to afford structure 31. Intramolecular palladium coupling reaction of structure 31 affords structure 32. Treatment of structure 32 with boron tribromide affords structure 33.

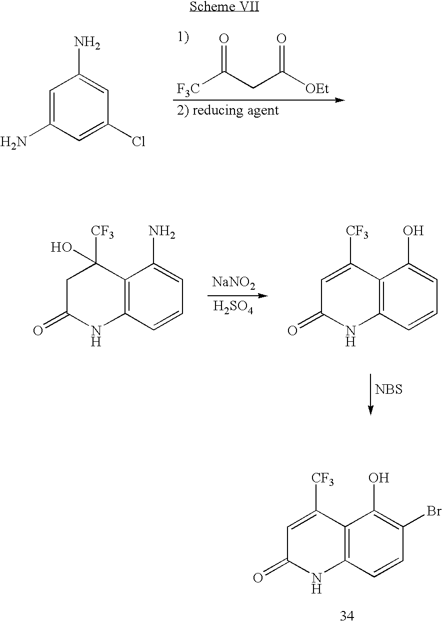

Scheme VII describes the synthesis of compound 34, as described in US 2002/0183314 (incorporated herein by reference). Generally, Knorr cyclization of a phenylenediamine, for example, 5-chloro-1,3-phenylenediamine, with a β-ketoester, for example, 4,4,4-trifluoroacetoacetate, affords the corresponding (1H)-quinolin-2-one. Reduction of the halide group could be achieved by chemical reduction with, for example, a metal catalyst, for example, palladium on carbon with a hydrogen atmosphere. Conversion of the aniline to a phenol could be effected by treatment with a diazotizing agent, for example, sodium nitrite in sulfuric acid. Brominatipn of the phenol, with a brominating agent, for example, N-bromosuccinimide, affords compound 34.

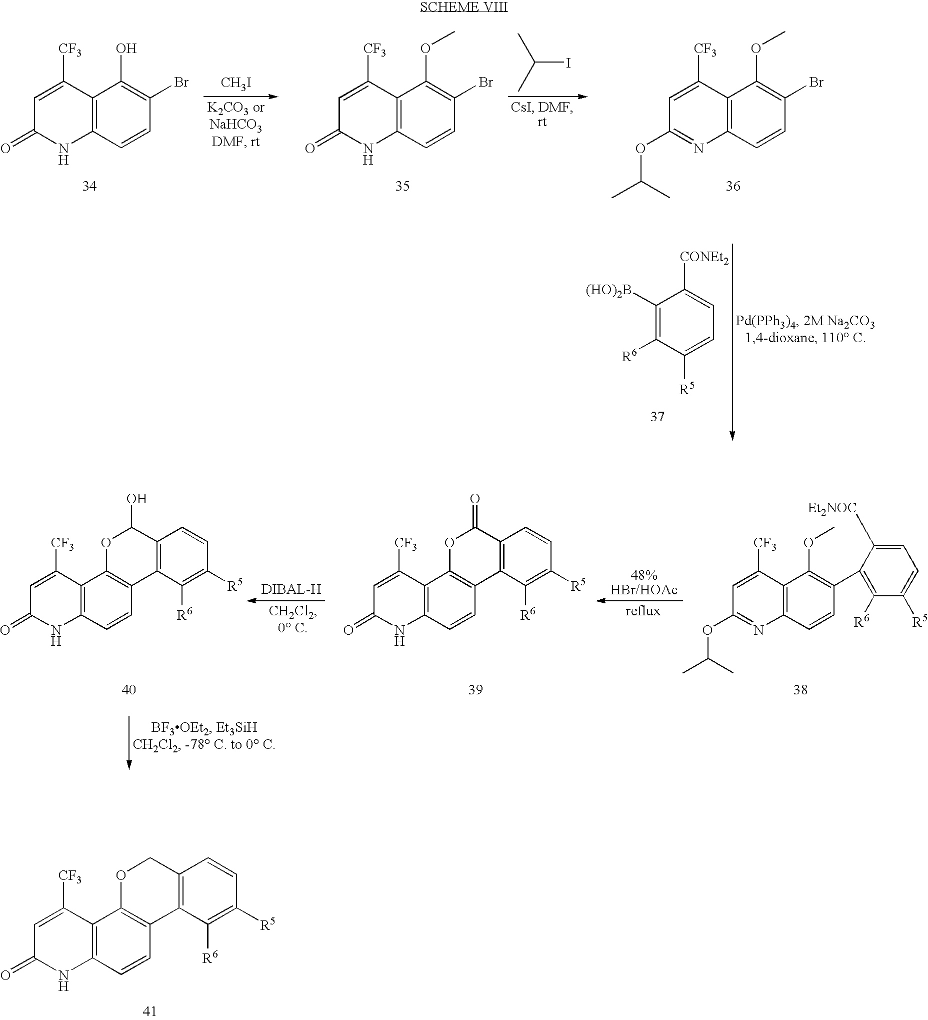

Scheme VIII describes the synthesis of tetracyclic quinolinone 41 from compound 34. Methylation of structure 34 with methyl iodide in the presence of a base affords structure 35, which undergoes alkylation to afford structure 36. Palladium coupling reaction of structure 36 and boronic acid 37 affords structure 38. Hydrolysis followed by cyclization of structure 38 in cone. HBr and AcOH affords structure 39. Reduction of structure 39 by diisobutylaluminum hydride affords structure 40. Treatment of structure 40 with triethylsilane in the presence of boron trifluoride etherate affords structure 41.

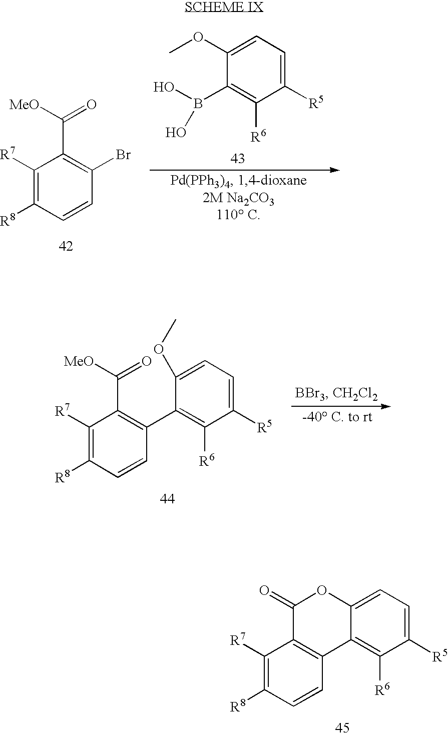

Scheme IX describes synthesis of benzochromenones of structure 45 from a bromobenzoate derivatives of structure 42. Palladium coupling reaction of structure 42 and boronic acid 43 affords intermediates of structure 44. Treatment of compounds of structure 44 with boron tribromide affords compounds of structure 45.

### D. Certain Indications

In certain embodiments, compounds and/or compositions provided herein are useful in the prevention, treatment, or amelioration of one or more of the symptoms of diseases or disorders associated with androgen receptor activity. Such prevention, treatment, or amelioration of diseases or disorders include, but are not limited to, maintenance of muscle strength and function (e.g., in the elderly); reversal or prevention of frailty or age-related functional decline (“ARFD”) in the elderly (e.g., sarcopenia); treatment of catabolic side effects of glucocorticoids; prevention and/or treatment of reduced bone mass, density or growth (e.g., osteoporosis and osteopenia); treatment of chronic fatigue syndrome (CFS); treatment of chronic myalgia; treatment of acute fatigue syndrome and muscle loss following elective surgery (e.g., post-surgical rehabilitation); accelerating wound healing; accelerating bone fracture repair (such as accelerating the recovery of hip fracture patients); accelerating healing of complicated fractures, e.g., distraction osteogenesis; in joint replacement; prevention of post-surgical adhesion formation; acceleration of tooth repair or growth; maintenance of sensory function (e.g., hearing, sight, olefaction and taste); treatment of periodontal disease; treatment of wasting secondary to fractures and wasting in connection with chronic obstructive pulmonary disease (COPD), chronic liver disease, AIDS, weightlessness, cancer cachexia, burn and trauma recovery, chronic catabolic state (e.g., coma), eating disorders (e.g., anorexia) and chemotherapy; treatment of cardiomyopathy; treatment of thrombocytopenia; treatment of growth retardation in connection with Crohn's disease; treatment of short bowel syndrome; treatment of irritable bowel syndrome; treatment of inflammatory bowel disease; treatment of Crohn's disease and ulcerative colitis; treatment of complications associated with transplantation; treatment of physiological short stature including growth hormone deficient children and short stature associated with chronic illness; treatment of obesity and growth retardation associated with obesity; treatment of anorexia (e.g., associated with cachexia or aging); treatment of hypercortisolism and Cushing's syndrome; treatment of Paget's disease; treatment of osteoarthritis; induction of pulsatile growth hormone release; treatment of osteochondrodysplasias; treatment of depression, nervousness, irritability and stress; treatment of reduced mental energy and low self-esteem (e.g., motivation/assertiveness); improvement of cognitive function (e.g., the treatment of dementia, including Alzheimer's disease and short term memory loss); treatment of catabolism in connection with pulmonary dysfunction and ventilator dependency; treatment of cardiac dysfunction (e.g., associated with valvular disease, myocardial infarction, cardiac hypertrophy or congestive heart failure); lowering blood pressure; protection against ventricular dysfunction or prevention of reperfusion events; treatment of adults in chronic dialysis; reversal or slowing of the catabolic state of aging; attenuation or reversal of protein catabolic responses following trauma (e.g., reversal of the catabolic state associated with surgery, congestive heart failure, cardiac myopathy, burns, cancer, COPD, etc.); reducing cachexia and protein loss due to chronic illness such as cancer or AIDS; treatment of hyperinsulinemia including nesidioblastosis; treatment of immunosuppressed patients; treatment of wasting in connection with multiple sclerosis or other neurodegenerative disorders; promotion of myelin repair; maintenance of skin thickness; treatment of metabolic homeostasis and renal homeostasis (e.g., in the frail elderly); stimulation of osteoblasts, bone remodeling and cartilage growth; regulation of food intake; treatment of insulin resistance, including NIDDM, in mammals (e.g., humans); treatment of insulin resistance in the heart; improvement of sleep quality and correction of the relative hyposomatotropism of senescence due to high increase in REM sleep and a decrease in REM latency; treatment of hypothermia; treatment of congestive heart failure; treatment of lipodystrophy (e.g., in patients taking HIV or AIDS therapies such as protease inhibitors); treatment of muscular atrophy (e.g., due to physical inactivity, bed rest or reduced weight-bearing conditions); treatment of musculoskeletal impairment (e.g., in the elderly); improvement of the overall pulmonary function; treatment of sleep disorders; treatment of the catabolic state of prolonged critical illness; treatment of hirsutism, acne, seborrhea, androgenic alopecia, anemia, hyperpilosity, benign prostate hypertrophy, adenomas and neoplasms of the prostate (e.g., advanced metastatic prostate cancer) and malignant tumor cells containing the androgen receptor, such as is the case for breast, brain, skin, ovarian, bladder, lymphatic, liver and kidney cancers; treatment of cancers of the skin, pancreas, endometrium, lung and colon; treatment of osteosarcoma; treatment of hypercalcemia of malignancy; treatment of metastatic bone disease; treatment of spermatogenesis, endometriosis and polycystic ovary syndrome; counteracting preeclampsia, eclampsia of pregnancy and preterm labor; treatment of premenstrual syndrome; treatment of vaginal dryness; treatment of age related decreased testosterone levels in men, male menopause, hypogonadism, male hormone replacement, male and female sexual dysfunction (e.g., erectile dysfunction, decreased sex drive, sexual well-being, decreased libido), male and female contraception, hair loss, Reaven's Syndrome and the enhancement of bone and muscle performance/strength.

In certain instances, prostate cancer is dependant on androgens. Such androgen dependent prostate cancer is typically amenable to treatment by androgen receptor antagonists and Vor androgen receptor partial agonists. In certain instances, prostate cancer is androgen independent. In such instances, androgen receptor antagonists are less effective or completely ineffective. However, in certain instances, androgen independent prostate cancer is androgen receptor dependant. In such instances, androgen receptor reducing compounds, including, but not limited to, androgen receptor degrading compounds can provide effective treatment. See, e.g., U.S. Pat. No. 6,861,432.

In certain embodiments, compounds and/or compositions provided herein are therapeutically effective for treating prostate cancer. In certain such embodiments, the prostate cancer is androgen dependant prostate cancer. In certain embodiments, the prostate cancer is androgen independent prostate cancer. In certain embodiments, the prostate cancer is androgen independent, but androgen receptor dependant prostate cancer. In certain such embodiments, administration of compounds and/or compositions provided herein results in a decrease in the amount of functional androgen receptor present in cells. In certain embodiments, administration of compositions provided herein results in degradation of androgen receptors.

### E. Formulation of Pharmaceutical Compositions

The pharmaceutical compositions provided herein contain therapeutically effective amounts of one or more of the androgen receptor activity modulators provided herein that are useful in the prevention, treatment, or amelioration of one or more of the symptoms of diseases or disorders associated with androgen receptor activity.

The compositions contain one or more compounds provided herein. The compounds are formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. Typically the compounds described above are formulated into pharmaceutical compositions using techniques and procedures well known in the art (see, e.g., Ansel, Introduction to Pharmaceutical Dosage Forms, Fourth Edition (1985), 126).

In certain embodiments, a pharmaceutical composition containing one or more compounds provided herein is prepared using known techniques, including, but not limited to mixing, dissolving, granulating, dragee-making, levigating, emulsifying, encapsulating, entrapping or tabletting processes.

In the compositions, effective concentrations of one or more compounds or pharmaceutically acceptable derivatives is (are) mixed with a suitable pharmaceutical carrier or vehicle. The compounds can be derivatized as the corresponding salts, esters, enol ethers or esters, acids, bases, solvates, hydrates or prodrugs prior to formulation, as described above. The concentrations of the compounds in the compositions are effective for delivery of an amount, upon administration, that treats, prevents, or ameliorates one or more of the symptoms of diseases or disorders associated with androgen activity or in which androgen activity is implicated.

Typically, the compositions are formulated for single dosage administration. To formulate a composition, the weight fraction of compound is dissolved, suspended, dispersed or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the particular mode of administration.

In addition, the compounds can be formulated as the sole pharmaceutically active ingredient in the composition or can be combined with other active ingredients. Liposomal suspensions, including tissue-targeted liposomes, such as tumor-targeted liposomes, can also be suitable as pharmaceutically acceptable carriers. These can be prepared by methods known to those skilled in the art. For example, liposome formulations can be prepared as described in U.S. Pat. No. 4,522,811. Briefly, liposomes such as multilamellar vesicles (MLV's) can be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

The active compound is included in the pharmaceutically acceptable carrier in an amount sufficient to exert a therapeutically useful effect in the absence of undesirable side effects on the patient treated.

The concentration of active compound in the pharmaceutical composition will depend on absorption, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of diseases or disorders associated with androgen activity or in which androgen activity is implicated, as described herein.

The effective amount of a compound provided herein can be determined empirically, if needed, by one of skill in the art. Exemplary dosage, include, amounts for a mammal of from about 0.05 to 100 mg/kg of body weight of active compound per day. Dosages can be administered in a single dose or in the form of individual divided doses, such as from 1 to 4 times per day. It will be understood that the specific dose level and frequency of dosage for any particular subject can be varied and will depend upon a variety of factors, including, for example, the activity of the specific compound employed, the metabolic stability and length of action of that compound, the species, age, body weight, general health, sex and diet of the subject, the mode and time of administration, rate of excretion, drug combination, and severity of the particular condition.

The active ingredient can be administered at once, or can be divided into a number of smaller doses to be administered at intervals of time. It is understood that the precise dosage and duration of treatment is a function of the disease being treated and can be determined empirically using known testing protocols or by extrapolation from in vivo or in vitro test data. It is to be noted that concentrations and dosage values can also vary with the severity of the condition to be alleviated. It is to be further understood that for any particular subject, specific dosage regimens should be adjusted over time of the individual need and the professional judgment of the person administering or supervising the administration of the compositions, and that the concentration ranges set forth herein are exemplary only and are not intended to limit the scope or practice of the compounds, compositions, methods and other subject matter provided herein.

Pharmaceutically acceptable derivatives include acids, bases, enol ethers and esters, salts, esters, hydrates, solvates and prodrug forms. The derivative is selected from among such that its pharmacokinetic properties are superior to the corresponding neutral compound.

Thus, effective concentrations or amounts of one or more of the compounds described herein or pharmaceutically acceptable derivatives thereof are mixed with a suitable pharmaceutical carrier or vehicle for systemic, topical or local administration to form pharmaceutical compositions. Compounds are included in an amount effective for ameliorating one or more symptoms of, or for treating or preventing diseases or disorders associated with androgen receptor activity or in which androgen receptor activity is implicated, as described herein. The concentration of active compound in the composition will depend on absorption, inactivation, excretion rates of the active compound, the dosage schedule, amount administered, particular formulation as well as other factors known to those of skill in the art.

The compositions are intended to be administered by a suitable route, including orally in form of capsules, tablets, granules, powders or liquid formulations including syrups; parenterally, such as subcutaneously, intravenously, intramuscularly, with intrasternal injection or infusion techniques (as sterile injectable aqueous or non-aqueous solutions or suspensions); nasally such as by inhalation spray; topically, such as in the form of a cream or ointment; rectally such as in the form of suppositories; liposomally; and locally. The compositions can be in liquid, semi-liquid or solid form and are formulated in a manner suitable for each route of administration. In certain embodiments, administration of the formulation includes parenteral and oral modes of administration. In one embodiment, the compositions are administered orally.

In certain embodiments, the pharmaceutical compositions provided herein containing one or more compounds provided herein is a solid (e.g., a powder, tablet, and/or capsule). In certain of such embodiments, a solid the pharmaceutical composition containing one or more compounds provided herein is prepared using ingredients known in the art, including, but not limited to, starches, sugars, diluents, granulating agents, lubricants, binders, and disintegrating agents.

In certain embodiments, a pharmaceutical composition containing one or more compounds provided herein is formulated as a depot preparation. Certain of such depot preparations are typically longer acting than non-depot preparations. In certain embodiments, such preparations are administered by implantation (for example subcutaneously or intramuscularly) or by intramuscular injection. In certain embodiments, depot preparations are prepared using suitable polymeric or hydrophobic materials (for example an emulsion in an acceptable oil) or ion exchange resins, or as sparingly soluble derivatives, for example, as a sparingly soluble salt.

In certain embodiments, a pharmaceutical composition containing one or more compounds provided herein contains a delivery system. Examples of delivery systems include, but are not limited to, liposomes and emulsions. Certain delivery systems are useful for preparing certain pharmaceutical compositions including those containing hydrophobic compounds. In certain embodiments, certain organic solvents such as dimethylsulfoxide are used.

In certain embodiments, a pharmaceutical composition containing one or more compounds provided herein contains one or more tissue-specific delivery molecules designed to deliver the pharmaceutical composition to specific tissues or cell types. For example, in certain embodiments, pharmaceutical compositions include liposomes coated with a tissue-specific antibody.

In certain embodiments, a pharmaceutical composition containing one or more compounds provided herein contains a co-solvent system. Certain of such co-solvent systems contain, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol containing 3% w/v benzyl alcohol, 8% w/v of the nonpolar surfactant Polysorbate 80™, and 65% w/v polyethylene glycol 300. The proportions of such co-solvent systems can be varied considerably without significantly altering their solubility and toxicity characteristics. Furthermore, the identity of co-solvent components can be varied: for example, other surfactants can be used instead of Polysorbate 80™; the fraction size of polyethylene glycol can be varied; other biocompatible polymers can replace polyethylene glycol, e.g., polyvinyl pyrrolidone; and other sugars or polysaccharides can substitute for dextrose.

In certain embodiments, solutions or suspensions used for parenteral, intradermal, subcutaneous, or topical application can include any of the following components: a sterile diluent, such as water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediamine-tetraacetic acid (EDTA); buffers, such as acetates, citrates and phosphates; and agents for the adjustment of tonicity such as sodium chloride or dextrose. Parenteral preparations can be enclosed in ampules, disposable syringes or single or multiple dose vials made of glass, plastic or other suitable material.

In instances in which the compounds exhibit insufficient solubility, methods for solubilizing compounds can be used. Such methods are known to those of skill in this art, and include, but are not limited to, using cosolvents, such as dimethylsulfoxide (DMSO), using surfactants, such as TWEEN®, or dissolution in aqueous sodium bicarbonate. Derivatives of the compounds, such as prodrugs of the compounds can also be used in formulating effective pharmaceutical compositions.

In certain embodiments, a pharmaceutical composition containing one or more compounds provided herein includes a sustained release system. A non-limiting example of such a sustained-release system is a semipermeable matrix of solid hydrophobic polymers. In certain embodiments, sustained release systems may, depending on their chemical nature, release compounds over a period of hours, days, weeks or months.

In certain embodiments, upon mixing or addition of the compound(s), the resulting mixture can be a solution, suspension, emulsion or the like. The form of the resulting mixture depends upon a number of factors, including the intended mode of administration and the solubility of the compound in the selected from among carrier or vehicle. The effective concentration is sufficient for ameliorating the symptoms of the disease, disorder or condition treated and can be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The pharmaceutically active compounds and derivatives thereof are typically formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes and individually packaged tablets or capsules. Unit-dose forms can be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

The composition can contain along with the active ingredient: a diluent such as lactose, sucrose, dicalcium phosphate, or carboxymethylcellulose; a lubricant, such as magnesium stearate, calcium stearate and talc; and a binder such as starch, natural gums, such as gum acacia, gelatin, glucose, molasses, polyinylpyrrolidine, celluloses and derivatives thereof, povidone, crospovidones and other such binders known to those of skill in the art. Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered can also contain minor amounts of nontoxic auxiliary substances such as wetting agents, emulsifying agents, or solubilizing agents, pH buffering agents and the like, for example, acetate, sodium citrate, cyclodextrin derivatives, sorbitan monolaurate, triethanolamine sodium acetate, triethanolamine oleate, and other such agents. Actual methods of preparing such dosage forms are known, or will be apparent, to those skilled in this art; for example, see Remington's Pharmaceutical Sciences, Mack Publishing Company, Easton, Pa., 15th Edition, 1975. The composition or formulation to be administered will, in any event, contain a quantity of the active compound in an amount sufficient to alleviate the symptoms of the treated subject.

Dosage forms or compositions containing active ingredient in the range of 0.005% to 100% with the balance made up from non-toxic carrier can be prepared. For oral administration, a pharmaceutically acceptable non-toxic composition is formed by the incorporation of any of the normally employed excipients, such as, for example pharmaceutical grades of mannitol, lactose, starch, magnesium stearate, talcum, cellulose derivatives, sodium crosscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art. The contemplated compositions can contain 0.001%-100% active ingredient, in one embodiment 0.1-85%, in another embodiment 75-95%.

In certain embodiments, the compounds can be administered in a form suitable for immediate release or extended release. Immediate release or extended release can be achieved with suitable pharmaceutical compositions or, particularly in the case of extended release, with devices such as subcutaneous implants or osmotic pumps. Exemplary compositions for topical administration include a topical carrier such as PLASTIBASE® (mineral oil gelled with polyethylene).

In certain embodiments, compounds used in the pharmaceutical compositions can be provided as pharmaceutically acceptable salts with pharmaceutically compatible counter ions. Pharmaceutically compatible salts can be formed with many acids, including but not limited to hydrochloric, sulfuric, acetic, lactic, tartaric, malic, succinic, etc.

In certain embodiments, the pharmaceutical compositions contain a compound provided herein in a therapeutically effective amount. In certain embodiments, the therapeutically effective amount is sufficient to prevent, alleviate or ameliorate symptoms of a disease or to prolong the survival of the subject being treated. Determination of a therapeutically effective amount is well within the capability of those skilled in the art.

The compositions can include other active compounds to obtain desired combinations of properties. The compounds provided herein, or pharmaceutically acceptable derivatives thereof as described herein, can also be advantageously administered for therapeutic or prophylactic purposes together with another pharmacological agent known in the general art to be of value in treating one or more of the diseases or medical conditions referred to hereinabove, such as diseases or disorders associated with androgen receptor activity or in which androgen receptor activity is implicated. It is to be understood that such combination therapy constitutes a further aspect of the compositions and methods of treatment provided herein.

In certain embodiments, a pharmaceutical composition containing one or more compounds provided herein is formulated as a prodrug. In certain embodiments, prodrugs are useful because they are easier to administer than the corresponding active form. For example, in certain instances, a prodrug can be more bioavailable (e.g., through oral administration) than is the corresponding active form. In certain instances, a prodrug can have improved solubility compared to the corresponding active form. In certain embodiments, a prodrug is an ester. In certain embodiments, such prodrugs are less water soluble than the corresponding active form. In certain instances, such prodrugs possess superior transmittal across cell membranes, where water solubility is detrimental to mobility. In certain embodiments, the ester in such prodrugs is metabolically hydrolyzed to carboxylic acid. In certain instances the carboxylic acid containing compound is the corresponding active form. In certain embodiments, a prodrug contains a short peptide (polyaminoacid) bound to an acid group. In certain of such embodiments, the peptide is metabolized to form the corresponding active form.

In certain embodiments, a pharmaceutical composition containing one or more compounds provided herein is useful for treating a condition or disorder in a mammalian, and particularly in a human patient. Suitable administration routes include, but are not limited to, oral, rectal, transmucosal, intestinal, enteral, topical, suppository, through inhalation, intrathecal, intraventricular, intraperitoneal, intranasal, intraocular and parenteral (e.g., intravenous, intramuscular, intramedullary, and subcutaneous). In certain embodiments, pharmaceutical compositions are administered to achieve local rather than systemic exposures. For example, pharmaceutical compositions can be injected directly in the area of desired effect (e.g., in the renal or cardiac area). In certain embodiments in which the pharmaceutical composition is administered locally, the dosage regimen is adjusted to achieve a desired local concentration of a compound provided herein.

In certain embodiments, a pharmaceutical composition containing one or more compounds provided herein is administered in the form of a dosage unit (e.g., tablet, capsule, bolus, etc.). In certain embodiments, such dosage units contain a selective androgen receptor modulator in a dose from about 1 μg/kg of body weight to about 50 mg/kg of body weight. In certain embodiments, such dosage units contain a selective androgen receptor modulator in a dose from about 2 μg/kg of body weight to about 25 mg/kg of body weight. In certain embodiments, such dosage units contain a selective androgen receptor modulator in a dose from about 10 μ/kg of body weight to about 5 mg/kg of body weight. In certain embodiments, pharmaceutical compositions are administered as needed, once per day, twice per day, three times per day, or four or more times per day. It is recognized by those skilled in the art that the particular dose, frequency, and duration of administration depends on a number of factors, including, without limitation, the biological activity desired, the condition of the patient, and tolerance for the pharmaceutical composition.

In certain embodiments, a pharmaceutical composition provided herein is administered for a period of continuous therapy. For example, a pharmaceutical composition provided herein can be administered over a period of days, weeks, months, or years.

Dosage amount, interval between doses, and duration of treatment can be adjusted to achieve a desired effect. In certain embodiments, dosage amount and interval between doses are adjusted to maintain a desired concentration on compound in a patient. For example, in certain embodiments, dosage amount and interval between doses are adjusted to provide plasma concentration of a compound provided herein at an amount sufficient to achieve a desired effect. In certain of such embodiments the plasma concentration is maintained above the minimal effective concentration (MEC). In certain embodiments, pharmaceutical compositions provided herein are administered with a dosage regimen designed to maintain a concentration above the MEC for 10-90% of the time, between 30-90% of the time, or between 50-90% of the time.

1. Compositions for Oral Administration

In certain embodiments, oral pharmaceutical dosage forms are either solid, gel or liquid. The solid dosage forms are tablets, capsules, granules, and bulk powders. Types of oral tablets include compressed, chewable lozenges and tablets which can be enteric-coated, sugar-coated or film-coated. Capsules can be hard or soft gelatin capsules, while granules and powders can be provided in non-effervescent or effervescent form with the combination of other ingredients known to those skilled in the art.

In certain embodiments, the formulations are solid dosage forms, preferably capsules or tablets. The tablets, pills, capsules, troches and the like can contain any of the following ingredients, or compounds of a similar nature: a binder; a diluent; a disintegrating agent; a lubricant; a glidant; a sweetening agent; and a flavoring agent.

In certain embodiments, pharmaceutical compositions for oral administration are push fit capsules made of gelatin. Certain of such push fit capsules contain one or more compounds provided herein in admixture with one or more filler such as lactose, binders such as starches, and/or lubricants such as talc or magnesium stearate and, optionally, stabilizers. In certain embodiments, pharmaceutical compositions for oral administration are soft, sealed capsules made of gelatin and a plasticizer, such as glycerol or sorbitol. In certain soft capsules, one or more compounds provided are dissolved or suspended in suitable liquids, such as fatty oils, liquid paraffin, or liquid polyethylene glycols. In addition, stabilizers can be added.

In certain embodiments, pharmaceutical compositions are prepared for buccal administration. Certain of such pharmaceutical compositions are tablets or lozenges formulated in conventional manner.

Examples of binders for use in the compositions provided herein include microcrystalline cellulose, gum tragacanth, glucose solution, acacia mucilage, gelatin solution, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include cross-carmellose sodium, sodium starch glycolate, alginic acid, sodium alginate, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

If oral administration is desired, the compound can be provided in a composition that protects it from the acidic environment of the stomach. For example, the composition can be formulated in an enteric coating that maintains its integrity in the stomach and releases the active compound in the intestine. The composition can also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup can contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a compound or pharmaceutically acceptable derivative thereof as described herein. Higher concentrations, up to about 98% by weight of the active ingredient can be included.

Pharmaceutically acceptable carriers included in tablets are binders, lubricants, diluents, disintegrating agents, coloring agents, flavoring agents, and wetting agents. Enteric-coated tablets, because of the enteric-coating, resist the action of stomach acid and dissolve or disintegrate in the neutral or alkaline intestines. Sugar-coated tablets are compressed tablets to which different layers of pharmaceutically acceptable substances are applied. Film-coated tablets are compressed tablets which have been coated with a polymer or other suitable coating. Multiple compressed tablets are compressed tablets made by more than one compression cycle utilizing the pharmaceutically acceptable substances previously mentioned. Coloring agents can also be used in the above dosage forms. Flavoring and sweetening agents are used in compressed tablets, sugar-coated, multiple compressed and chewable tablets. Flavoring and sweetening agents are especially useful in the formation of chewable tablets and lozenges.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and can contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic add, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Diluents include lactose and sucrose. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic adds include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is preferably encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, can be diluted with a sufficient quantity of a pharmaceutically acceptable liquid carrier, e.g., water, to be easily measured for administration.

Alternatively, liquid or semi-solid oral formulations can be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. Re 28,819 and 4,358,603. Briefly, such formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl)acetals of lower alkyl aldehydes such as acetaldehyde diethyl acetal.

In all embodiments, tablets and capsules formulations can be coated as known by those of skill in the art in order to modify or sustain dissolution of the active ingredient. Thus, for example, they can be coated with a conventional enterically digestible coating, such as phenylsalicylate, waxes and cellulose acetate phthalate.

Exemplary compositions can include fast-dissolving diluents such as mannitol, lactose, sucrose, and/or cyclodextrins. Also included in such formulations can be high molecular weight excipients such as celluloses (AVICEL®) or polyethylene glycols (PEG); an excipient to aid mucosal adhesion such as hydroxypropyl cellulose (HPC), hydroxypropyl methyl cellulose (HPMC), sodium carboxymethyl cellulose (SCMC), and/or maleic anhydride copolymer (e.g., GANTREZ®); and agents to control release such as polyacrylic copolymer (e.g., CARBOPOL 934®). Lubricants, glidants, flavors, coloring agents and stabilizers can also be added for ease of fabrication and use.

In certain of such embodiments, a pharmaceutical composition for oral administration is formulated by combining one or more compounds provided herein with one or more pharmaceutically acceptable carriers. Certain of such carriers enable compounds provided herein to be formulated as tablets, pills, dragees, capsules, liquids, gels, syrups, slurries, suspensions and the like, for oral ingestion by a patient. In certain embodiments, pharmaceutical compositions for oral use are obtained by mixing one or more compounds provided herein and one or more solid excipient. Suitable excipients include, but are not limited to, fillers, such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethyl-cellulose, and/or polyvinylpyrrolidone (PVP). In certain embodiments, such a mixture is optionally ground and auxiliaries are optionally added. In certain embodiments, pharmaceutical compositions are formed to obtain tablets or dragee cores. In certain embodiments, disintegrating agents (e.g., cross linked polyvinyl pyrrolidone, agar, or alginic acid or a salt thereof, such as sodium alginate) are added.

In certain embodiments, dragee cores are provided with coatings. In certain of such embodiments, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to tablets or dragee coatings.

In certain embodiments, a daily dosage regimen for a patient contains an oral dose of between 0.1 mg and 2000 mg of a compound provided herein. In certain embodiments, a daily dosage regimen is administered as a single daily dose. In certain embodiments, a daily dosage regimen is administered as two, three, four, or more than four doses.

2. Injectables, Solutions and Emulsions

In certain embodiments, the pharmaceutical composition is prepared for transmucosal administration. In certain of such embodiments penetrants appropriate to the barrier to be permeated are used in the formulation. Such penetrants are generally known in the art.

Parenteral administration, generally characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol, mannitol, 1,3-butanediol, Ringer's solution, an isotonic sodium chloride solution or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents, such as for example, mono- or diglycerides, fatty acids, such as oleic acid, sodium acetate, sorbitan monolaurate, triethanolamine oleate and cyclodextrins. Implantation of a slow-release or sustained-release system, such that a constant level of dosage is maintained (see, e.g., U.S. Pat. No. 3,710,795) is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethylene-terephthalate, natural rubber, polyisoprene, polyisobutylene, polybutadiene, polyethylene, ethylene-vinyl acetate copolymers, silicone rubbers, polydimethyl-siloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions can be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethyl-cellulose, hydroxypropyl methylcellulose and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art.

The unit-dose parenteral preparations are packaged in an ampoule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. Typically a therapeutically effective dosage is formulated to contain a concentration of at least about 0.1% w/w up to about 90% w/w or more, preferably more than 1% w/w of the active compound to the treated tissue(s). The active ingredient can be administered at once, or can be divided into a number of smaller doses to be administered at intervals of time. It is understood that the precise dosage and duration of treatment is a function of the tissue being treated and can be determined empirically using known testing protocols or by extrapolation from in vivo or in vitro test data. It is to be noted that concentrations and dosage values can also vary with the age of the individual treated. It is to be further understood that for any particular subject, specific dosage regimens should be adjusted over time of the individual need and the professional judgment of the person administering or supervising the administration of the formulations, and that the concentration ranges set forth herein are exemplary only and are not intended to limit the scope or practice of formulations provided herein.

The compounds can be formulated in any suitable vehicle or form. For example, they can be in micronized or other suitable form and/or can be derivatized to produce a more soluble active product or to produce a prodrug or for other purposes. The form of the resulting mixture depends upon a number of factors, including, for example, an intended mode of administration and the solubility of the compound in the selected carrier or vehicle. The effective concentration is sufficient for ameliorating the symptoms of the condition and can be empirically determined.

In certain embodiments, a pharmaceutical composition is prepared for administration by injection wherein the pharmaceutical composition contains a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampules or in multi dose containers. Certain pharmaceutical compositions for injection are suspensions, solutions or emulsions in oily or aqueous vehicles, and can contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions can contain substances that increase the viscosity of the suspension, such as sodium carboxymethyl cellulose, sorbitol, or dextran. Optionally, such suspensions can also contain suitable stabilizers or agents that increase the solubility of the compounds to allow for the preparation of highly concentrated solutions.

In certain embodiments, the pharmaceutical composition is prepared for administration by inhalation. Certain of such pharmaceutical compositions for inhalation are prepared in the form of an aerosol spray in a pressurized pack or a nebulizer. Certain of such pharmaceutical compositions contain a propellant, e.g., dichlorodifluoromethane, trichlorofluoromethane, dichlorotetrafluoroethane, carbon dioxide or other suitable gas. In certain embodiments using a pressurized aerosol, the dosage unit can be determined with a valve that delivers a metered amount. In certain embodiments, capsules and cartridges for use in an inhaler or insufflator can be formulated. Certain of such formulations contain a powder mixture of a compound provided herein and a suitable powder base such as lactose or starch.

In certain embodiments, the pharmaceutical compositions provided are administered by continuous intravenous infusion. In certain of such embodiments, from 0.1 mg to 500 mg of the composition is administered per day.

3. Lyophilized Powders

Of interest herein are also lyophilized powders, which can be reconstituted for administration as solutions, emulsions and other mixtures. They can also be reconstituted and formulated as solids or gels.

The sterile, lyophilized powder is prepared by dissolving a compound provided herein, or a pharmaceutically acceptable derivative thereof, in a suitable solvent. The solvent can contain an excipient which improves the stability or other pharmacological component of the powder or reconstituted solution, prepared from the powder. Excipients that can be used include, but are not limited to, dextrose, sorbital, fructose, corn syrup, xylitol, glycerin, glucose, sucrose or other suitable agent. The solvent can also contain a buffer, such as citrate, sodium or potassium phosphate or other such buffer known to those of skill in the art at, typically, about neutral pH. Subsequent sterile filtration of the solution followed by lyophilization under standard conditions known to those of skill in the art provides the desired formulation. Generally, the resulting solution will be apportioned into vials for lyophilization. Each vial will contain a single dosage 10-1000 mg, in one embodiment, 100-500 mg or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, preferably 5-35 mg, more preferably about 9-30 mg of lyophilized powder, is added per mL of sterile water or other suitable carrier. The precise amount depends upon the selected compound. Such amount can be empirically determined.

4. Topical Administration

Topical mixtures are prepared as described for the local and systemic administration. The resulting mixture can be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable derivatives thereof can be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will typically have diameters of less than 50 microns, preferably less than 10 microns.

In certain embodiments, the pharmaceutical compositions for inhalation are prepared in the form of an aerosol spray in a pressurized pack or a nebulizer. Certain of such pharmaceutical compositions contain a propellant, e.g., dichlorodifluoro-methane, trichlorofluoromethane, dichlorotetrafluoroethane, carbon dioxide or other suitable gas. In certain embodiments using a pressurized aerosol, the dosage unit can be determined with a valve that delivers a metered amount. In certain embodiments, capsules and cartridges for use in an inhaler or insufflator can be formulated. Certain of such formulations contain a powder mixture of a compound provided herein and a suitable powder base such as lactose or starch.

Exemplary compositions for nasal aerosol or inhalation administration include solutions which can contain, for example, benzyl alcohol or other suitable preservatives, absorption promoters to enhance absorption and/or bioavailability, and/or other solubilizing or dispersing agents such as those known in the art.

The compounds can be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered. These solutions, particularly those intended for ophthalmic use, can be formulated as 0.01%-10% isotonic solutions, pH about 5-7, with appropriate salts. In certain embodiments in which the compositions are administered locally, the dosage regimen is adjusted to achieve a desired local concentration of a compound provided herein.

In certain embodiments, the pharmaceutical composition is prepared for topical administration. Certain of such pharmaceutical compositions contain bland moisturizing bases, such as ointments or creams. Exemplary suitable ointment bases include, but are not limited to, petrolatum, petrolatum plus volatile silicones, lanolin and water in oil emulsions such as Eucerin™, available from Beiersdorf (Cincinnati, Ohio). Exemplary suitable cream bases include, but are not limited to, Nivea™ Cream, available from Beiersdorf (Cincinnati, Ohio), cold cream (USP), Purpose Cream™, available from Johnson & Johnson (New Brunswick, N.J.), hydrophilic ointment (USP) and Lubriderm™, available from Pfizer (Morris Plains, N.J.).

In certain embodiments, the formulation, route of administration and dosage for the pharmaceutical composition provided herein can be chosen in view of a particular patient's condition. (See, e.g., Fingl et al., 1975, in “The Pharmacological Basis of Therapeutics”, Ch. 1, p. 1). In certain embodiments, the pharmaceutical composition is administered as a single dose. In certain embodiments, a pharmaceutical composition is administered as a series of two or more doses administered over one or more days.

5. Compositions for Other Routes of Administration

Other routes of administration, such as topical application, transdermal patches, and rectal administration are also contemplated herein.

In certain embodiments, the pharmaceutical composition is prepared for topical administration such as rectal administration. The pharmaceutical dosage forms for rectal administration include, but are not limited to rectal suppositories, capsules and tablets for systemic effect. In certain embodiments a pharmaceutical agent is prepared for rectal administration, such as a suppositories or retention enema. Certain of such pharmaceutical agents contain known ingredients, such as cocoa butter and/or other glycerides. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases can be used. In certain embodiments, the pharmaceutical compositions contain bland moisturizing bases, such as ointments or creams. Exemplary suitable ointment bases include, but are not limited to, petrolatum, petrolatum plus volatile silicones, lanolin and water in oil emulsions such as Eucerin®, available from Beiersdorf (Cincinnati, Ohio). Exemplary suitable cream bases include, but are not limited to, Nivea™ Cream, available from Beiersdorf (Cincinnati, Ohio), cold cream (USP), Purpose Cream™, available from Johnson & Johnson (New Brunswick, N.J.), hydrophilic ointment (USP) and Lubriderm™, available from Pfizer (Morris Plains, N.J.). Agents to raise the melting point of suppositories include spermaceti and wax. Rectal suppositories can be prepared either by the compressed method or by molding. The typical weight of a rectal suppository is about 2 to 3 gm.

Tablets and capsules for rectal administration are manufactured using the same pharmaceutically acceptable substance and by the same methods as for formulations for oral administration.

6. Articles of Manufacture

The compounds or pharmaceutically acceptable derivatives can be packaged as articles of manufacture containing packaging material, within the packaging material a compound or pharmaceutically acceptable derivative thereof provided herein, which is effective for modulating the activity of androgen receptor, or for treatment, prevention or amelioration of one or more symptoms of androgen receptor mediated diseases or disorders, or diseases or disorders in which androgen receptor activity is implicated, and a label that indicates that the compound or composition, or pharmaceutically acceptable derivative thereof, is used for modulating the activity of androgen receptor or for treatment, prevention or amelioration of one or more symptoms of androgen receptor mediated diseases or disorders, or diseases or disorders in which androgen receptor activity is implicated.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatments for any disease or disorder in which androgen receptor activity is implicated as a mediator or contributor to the symptoms or cause.

In certain embodiments, the pharmaceutical compositions can be presented in a pack or dispenser device which can contain one or more unit dosage forms containing a compound provided herein. The pack can for example contain metal or plastic foil, such as a blister pack. The pack or dispenser device can be accompanied by instructions for administration. The pack or dispenser can also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert. Compositions containing a compound provided herein formulated in a compatible pharmaceutical carrier can also be prepared, placed in an appropriate container, and labeled for treatment of an indicated condition.

### F. Evaluation of the Activity of the Compounds

Standard physiological, pharmacological and biochemical procedures are available for testing the compounds provided herein to identify those that possess activity as androgen receptor modulators. In vitro and in vivo assays known in the art can be used to evaluate the activity of the compounds provided herein as androgen receptor modulators. Exemplary assays include, but are not limited to, fluorescence polarization assay, luciferase assay, co-transfection assay. In certain embodiments, the compounds provided herein are capable of modulating activity of androgen receptor in a “co-transfection” assay (also called a “cis-trans” assay), which is known in the art. See, e.g., Evans et al, Science, 240:889-95 (1988); U.S. Pat. Nos. 4,981,784 and 5,071,773; Pathirana et al, “Nonsteroidal Human Progesterone Receptor Modulators from the Marie Alga Cymopolia Barbata,” Mol. Pharm. 47:630-35 (1995)). Modulating activity in a co-transfection assay has been shown to correlate with in vivo modulating activity. Thus, in certain embodiments, such assays are predictive of in vivo activity. See, e.g., Berger et al., J. Steroid Biochem. Molec. Biol. 41:773 (1992).

In certain co-transfection assays, two different co-transfection plasmids are prepared. In the first co-transfection plasmid, cloned cDNA encoding an intracellular receptor (e.g., androgen receptor) is operatively linked to a constitutive promoter (e.g., the SV 40 promoter). In the second co-transfection plasmid, cDNA encoding a reporter protein, such as firefly luciferase (LUC), is operatively linked to a promoter that is activated by a receptor-dependant activation factor. Both co-transfection plasmids are co-transfected into the same cells. Expression of the first co-transfection plasmid results in production of the intracellular receptor protein. Activation of that intracellular receptor protein (e.g., by binding of an agonist) results in production of a receptor-dependant activation factor for the promoter of the second co-transfection plasmid. That receptor-dependant activation factor in turn results in expression of the reporter protein encoded on the second co-transfection plasmid. Thus, reporter protein expression is linked to activation of the receptor. Typically, that reporter activity can be conveniently measured (e.g., as increased luciferase production).

Certain co-transfection assays can be used to identify agonists, partial agonists, and/or antagonists of intracellular receptors. In certain embodiments, to identify agonists, co-transfected cells are exposed to a test compound. If the test compound is an agonist or partial agonist, reporter activity is expected to increase compared to co-transfected cells in the absence of the test compound. In certain embodiments, to identify antagonists, the cells are exposed to a known agonist (e.g., androgen for the androgen receptor) in the presence and absence of a test compound. If the test compound is an antagonist, reporter activity is expected to decrease relative to that of cells exposed only to the known agonist.

In certain embodiments, compounds provided herein are used to detect the presence, quantity and/or state of receptors in a sample. In certain of such embodiments, samples are obtained from a patient. In certain embodiments, compounds are radio- or isotopically-labeled. For example, compounds provided herein that selectively bind androgen receptors can be used to determine the presence of such receptors in a sample, such as cell homogenates and lysates.

In certain embodiments, compounds provided herein are selective androgen receptor reducing compounds. In certain embodiments, compounds provided herein are selective androgen receptor degrading compounds. Assays for measuring the amount of androgen receptor are known in the art (e.g., see U.S. Pat. No. 6,861,432).

For example, the following is a model assay that is used to measure the content of androgen receptor protein in cells of the human prostate cell line LNCaP. In a 25 cm² cell culture flask, 2×10⁶ LNCaP cells in 6 ml of RPMI 1640 cell culture medium without phenol red are grown with 4 mmol of glutamine and 5% activated-carbon-treated serum (CCS) and are cultivated overnight at 37° C., 5% CO₂, in a moist atmosphere. On the next day, the cells are treated with the test substance at a concentration of 10 or 1 μm, whereby the final concentration of the solvent is 0.5% DMSO. As a control, cells are treated only with 0.5% DMSO. After an incubation time of 24 hours, the medium is changed with a renewed addition of substance and another 24 hours of incubation. After 48 hours, the cells are washed with PBS, dissolved with PBS/20 mmol of EDTA, washed again with PBS-CA²⁺/Mg²⁺ and then frozen for at least two hours as cell pellets at −80° C. Then, the cell pellet is resuspended in 200 μl of lysis buffer (50 mmol of tris/HCl, pH 7.5; 150 mmol of NaCl, 1.5 mmol of MgCl₂, 0.2% SDS, 10% glycerol, 1 mmol of DTT, 0.01× complete-EDTA protease inhibitors (Roche, Mannheim)) and treated with 10 U benzonase (Merck, Darnstadt) for 10 minutes at 4° C. After this time, 5 mmol of EDTA is added, insoluble material is pelletized, and 25 μg of the cell extract is separated in a 4-12% SDS-polyacrylamide gel (Invitrogen). Then, the proteins are transferred to nitrocellulose (HyBondECL, Amersham) and incubated with monoclonal antibodies against the androgen receptor (AR441; Santa Cruz Biotechnologies; 1:400 dilution) and actin (ICN, 1:5000-1:20,000 dilution). After incubation with the secondary antibodies (anti-mouse IgG-HRP, Amersham or -AP, Invitrogen), the Western blot is developed by chemiluminescence (ECL, Amersham; Western Breeze, Invitrogen), and the light signals are quantified with a Chemilmager™ (Kodak). The amount of androgen receptor is calculated in a ratio to actin as a percentage of the DMSO control.

### G. Methods of Use of the Compounds and Compositions

Methods of use of the compounds and compositions provided herein also are provided. The methods include in vitro and in vivo uses of the compounds and compositions for altering androgen receptor activity and for treatment, prevention, or amelioration of one or more symptoms of diseases or disorders that are modulated by androgen receptor activity, or in which androgen receptor activity, is implicated. In certain embodiments, provided herein are methods of treating a patient by administering a compound provided herein. In certain embodiments, such patient exhibits symptoms or signs of an androgen receptor mediated condition. In certain embodiments, a patient is treated prophylactically to reduce or prevent the occurrence of a condition.

The compounds provided herein can be used in the treatment of a variety of conditions including, but not limited to, maintenance of muscle strength and function (e.g., in the elderly); reversal or prevention of frailty or age-related functional decline (“ARFD”) in the elderly (e.g., sarcopenia); treatment of catabolic side effects of glucocorticoids; prevention and/or treatment of reduced bone mass, density or growth (e.g., osteoporosis and osteopenia); treatment of chronic fatigue syndrome (CFS); treatment of chronic myalgia; treatment of acute fatigue syndrome and muscle loss following elective surgery (e.g., post-surgical rehabilitation); accelerating wound healing; accelerating bone fracture repair (such as accelerating the recovery of hip fracture patients); accelerating healing of complicated fractures, e.g., distraction osteogenesis; in joint replacement; prevention of post-surgical adhesion formation; acceleration of tooth repair or growth; maintenance of sensory function (e.g., hearing, sight, olefaction and taste); treatment of periodontal disease; treatment of wasting secondary to fractures and wasting in connection with chronic obstructive pulmonary disease (COPD), chronic liver disease, AIDS, weightlessness, cancer cachexia, burn and trauma recovery, chronic catabolic state (e.g., coma), eating disorders (e.g., anorexia) and chemotherapy; treatment of cardiomyopathy; treatment of thrombo-cytopenia; treatment of growth retardation in connection with Crohn's disease; treatment of short bowel syndrome; treatment of irritable bowel syndrome; treatment of inflammatory bowel disease; treatment of Crohn's disease and ulcerative colitis; treatment of complications associated with transplantation; treatment of physiological short stature including growth hormone deficient children and short stature associated with chronic illness; treatment of obesity and growth retardation associated with obesity; treatment of anorexia (e.g., associated with cachexia or aging); treatment of hypercortisolism and Cushing's syndrome; treatment of Paget's disease; treatment of osteoarthritis; induction of pulsatile growth hormone release; treatment of osteochondrodysplasias; treatment of depression, nervousness, irritability and stress; treatment of reduced mental energy and low self-esteem (e.g., motivation/assertiveness); improvement of cognitive function (e.g., the treatment of dementia, including Alzheimer's disease and short term memory loss); treatment of catabolism in connection with pulmonary dysfunction and ventilator dependency; treatment of cardiac dysfunction (e.g., associated with valvular disease, myocardial infarction, cardiac hypertrophy or congestive heart failure); lowering blood pressure; protection against ventricular dysfunction or prevention of reperfusion events; treatment of adults in chronic dialysis; reversal or slowing of the catabolic state of aging; attenuation or reversal of protein catabolic responses following trauma (e.g., reversal of the catabolic state associated with surgery, congestive heart failure, cardiac myopathy, burns, cancer, COPD, etc.); reducing cachexia and protein loss due to chronic illness such as cancer or AIDS; treatment of hyperinsulinemia including nesidioblastosis; treatment of immunosuppressed patients; treatment of wasting in connection with multiple sclerosis or other neurodegenerative disorders; promotion of myelin repair; maintenance of skin thickness; treatment of metabolic homeostasis and renal homeostasis (e.g., in the frail elderly); stimulation of osteoblasts, bone remodeling and cartilage growth; regulation of food intake; treatment of insulin resistance, including NIDDM, in mammals (e.g., humans); treatment of insulin resistance in the heart; improvement of sleep quality and correction of the relative hyposomatotropism of senescence due to high increase in REM sleep and a decrease in REM latency; treatment of hypothermia; treatment of congestive heart failure; treatment of lipodystrophy (e.g., in patients taking HIV or AIDS therapies such as protease inhibitors); treatment of muscular atrophy (e.g., due to physical inactivity, bed rest or reduced weight-bearing conditions); treatment of musculoskeletal impairment (e.g., in the elderly); improvement of the overall pulmonary function; treatment of sleep disorders; treatment of the catabolic state of prolonged critical illness; treatment of hirsutism, acne, seborrhea, androgenic alopecia, anemia, hyperpilosity, benign prostate hypertrophy, adenomas and neoplasms of the prostate (e.g., advanced metastatic prostate cancer) and malignant tumor cells containing the androgen receptor, such as is the case for breast, brain, skin, ovarian, bladder, lymphatic, liver and kidney cancers; treatment of cancers of the skin, pancreas, endometrium, lung and colon; osteosarcoma; treatment of hypercalcemia of malignancy; treatment of metastatic bone disease; treatment of spermatogenesis, endometriosis and polycystic ovary syndrome; counteracting preeclampsia, eclampsia of pregnancy and preterm labor; treatment of premenstrual syndrome; treatment of vaginal dryness; treatment of age related decreased testosterone levels in men, male menopause, hypogonadism, male hormone replacement, male and female sexual dysfunction (e.g., erectile dysfunction, decreased sex drive, sexual well-being, decreased libido), male and female contraception, hair loss, Reaven's Syndrome and the enhancement of bone and muscle performance/strength. The term treatment is also intended to include prophylactic treatment.

In certain embodiments, the compounds provided herein are used to treat acne, male-pattern baldness, wasting diseases, hirsutism, hypogonadism, osteoporoses, infertility, impotence, obesity, and cancer. In certain embodiments, one or more compounds provided herein are used to stimulate hematopoiesis. In certain embodiments, one or more compounds provided herein are used for contraception.

In certain embodiments, one or more compounds provided herein are used to treat cancer. Certain exemplary cancers include, but are not limited to, breast cancer, colorectal cancer, gastric carcinoma, glioma, head and neck squamous cell carcinoma, papillary renal carcinoma, leukemia, lymphoma, Li-Fraumeni syndrome, malignant pleural mesothelioma, melanoma, multiple myeloma, non-small cell lung cancer, synovial sarcoma, thyroid carcinoma, transitional cell carcinoma of urinary bladder, and prostate cancer, including, but not limited to prostatic hyperplasia.

In certain embodiments, one or more compounds provided herein are used to improve athletic performance. In certain such embodiments, one or more compounds provided herein are used, for example to shorten the time normally needed to recover from physical exertion or to increase muscle strength. Athletes to whom one or more compounds provided herein can be administered include, but are not limited to, horses, dogs, and humans. In certain embodiments, one or more compounds provided herein are administered to an athlete engaged in a professional or recreational competition, including, but not limited to weight-lifting, body-building, track and field events, and any of various team sports.

In certain embodiments, provided are methods for treating a patient by administering one or more selective androgen receptor agonists and/or partial agonists. Exemplary conditions that can be treated with such selective androgen receptor agonists and/or partial agonist include, but are not limited to, hypogonadism, wasting diseases, cancer cachexia, frailty, infertility, and osteoporosis. In certain embodiments, a selective androgen receptor agonist or partial agonist is used for male hormone replacement therapy. In certain embodiments, one or more selective androgen receptor agonists and/or partial agonists are used to stimulate hematopoiesis. In certain embodiments, a selective androgen receptor agonist or partial agonist is used as an anabolic agent. In certain embodiments, a selective androgen receptor agonist and/or partial agonist is used to improve athletic performance.

In certain embodiments, provided herein are methods for treating a patient by administering one or more selective androgen receptor antagonists and/or partial agonists. Exemplary conditions that can be treated with such one or more selective androgen receptor antagonists and/or partial agonists include, but are not limited to, hirsutism, acne, male-pattern baldness, prostatic hyperplasia, and cancer, including, but not limited to, various hormone-dependent cancers, including, without limitation, prostate and breast cancer.

In certain embodiments, provided herein are methods for treating a patient with prostate cancer. In certain such embodiments, the prostate cancer is androgen dependant prostate cancer. In certain embodiments, the prostate cancer is androgen independent prostate cancer. In certain embodiments, the prostate cancer is androgen independent, but androgen receptor dependant prostate cancer. In certain such embodiments, administration of compositions provided herein results in a decrease in the amount of functional androgen receptor present in cells. In certain embodiments, administration of compositions provided herein results in degradation of androgen receptors.

### H. Combination Therapies

In certain embodiments, one or more compounds or compositions provided herein can be co-administered with one or more other pharmaceutical agents. In certain embodiments, such one or more other pharmaceutical agents are designed to treat the same disease or condition as the one or more compounds or pharmaceutical compositions provided herein. In certain embodiments, such one or more other pharmaceutical agents are designed to treat a different disease or condition as the one or more compounds or compositions provided herein. In certain embodiments, such one or more other pharmaceutical agents are designed to treat an undesired effect of one or more compounds or compositions provided herein. In certain embodiments, one or more compounds or compositions provided herein is co-administered with another pharmaceutical agent to treat an undesired effect of that other pharmaceutical agent.

In certain embodiments, compounds or compositions provided herein and one or more other pharmaceutical agents are administered at the same time. In certain embodiments, compounds or compositions provided herein and one or more other pharmaceutical agents are administered at different times. In certain embodiments, compounds or compositions provided herein and one or more other pharmaceutical agents are prepared together in a single formulation. In certain embodiments, compounds or compositions provided herein and one or more other pharmaceutical agents are prepared separately.

Examples of pharmaceutical agents that can be co-administered with compounds or compositions provided herein include, but are not limited to, analgesics (e.g., acetaminophen); anti-inflammatory agents, including, but not limited to non-steroidal anti-inflammatory drugs (e.g., ibuprofen, COX-1 inhibitors, and COX-2, inhibitors); salicylates; antibiotics; antivirals; antifungal agents; antidiabetic agents (e.g., biguanides, glucosidase inhibitors, insulins, sulfonylureas, and thiazolidene-diones); adrenergic modifiers; diuretics; hormones (e.g., anabolic steroids, androgen, estrogen, calcitonin, progestin, somatostan, and thyroid hormones); muscle relaxants; immunomodulators; antihistamines; osteoporosis agents (e.g., biphosphonates, calcitonin, and estrogens); prostaglandins, antineoplastic agents; psychotherapeutic agents; sedatives; poison oak or poison sumac products; antibodies; and vaccines.

In certain embodiments, pharmaceutical agents that can be co-administered with compounds or compositions provided herein include, but are not limited to, other modulators of nuclear hormone receptors or other suitable therapeutic agents useful in the treatment of the aforementioned disorders including: anti-diabetic agents; anti-osteoporosis agents; anti-obesity agents; anti-inflammatory agents; anti-anxiety agents; anti-depressants; anti-hypertensive agents; anti-platelet agents; anti-thrombotic and thrombolytic agents; cardiac glycosides; cholesterol/lipid lowering agents; mineralocorticoid receptor antagonists; phosphodiesterase inhibitors; protein tyrosine kinase inhibitors; thyroid mimetics (including thyroid receptor agonists); anabolic agents; HIV or AIDS therapies; therapies used in the treatment of Alzheimer's and other cognitive disorders; therapies used in the treatment of sleeping disorders; anti-proliferative agents; and anti-tumor agents.

### EXAMPLES

The following examples, including experiments and results achieved, are provided for illustrative purposes only and are not to be construed as limiting the scope of any claimed subject matter.

### Example 1

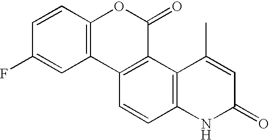

### 9-Fluoro-4-methyl-1H-6-oxa-1-azachrysene-2,5-dione (Compound 101, Structure 3 of Scheme II, where R²=methyl, R⁵=F, R⁶=H)

To a mixture of 1 and 4-dimethylaminopyridine in DMF was added 3-butenoyl chloride at rt. The reaction mixture was stirred at rt for 1 hr and the mixture was concentrated. Chromatography of the crude mixture afforded compound 2.

A mixture of compound 2, Pd(OAc)₂, (o-tolyl)₃P, and Et₃N in DMF was degassed, stirred at 90° C. for 24 hrs, and was concentrated. Chromatography of the crude mixture afforded Compound 101 as a white solid. ¹H NMR (500 MHz, CDCl₃) 12.18 (s, 1H), 8.54 (d, J=8.5 Hz, 1H), 8.25 (dd, J=9.5, 2.7 Hz, 1H), 7.81 (d, J=8.5 Hz, 1H), 7.52 (dd, J=9.0, 5.0 Hz, 1H), 7.44 (td, J=8.5, 3.0 Hz, 1H), 6.63 (s, 1H), 2.44 (d, J=0.5 Hz, 3H).

### Example 2

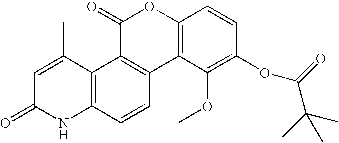

### 2,2-Dimethylpropionic acid 10-methoxy-4-methyl-2,5-dioxo-2,5-dihydro-1H-6-oxa-1-aza-chrysen-9-yl ester (Compound 102, Structure 3 of Scheme II, where R²=methyl, R⁵=O—CO-t-Bu, R⁶=OCH₃)

Compound 102 was synthesized in a similar fashion to that described in Example 1 as a white solid. ¹H NMR (500 MHz, CDCl₃) 12.71 (s, 1H), 9.08 (d, J=9.0 Hz, 1H), 7.87 (d, J=9.0 Hz, 1H), 7.25 (d, J=8.9 Hz, 1H), 7.22 (d, J=8.9 Hz, 1H), 6.84 (s, 1H), 3.87 (s, 3H), 2.64 (s, 3H), 1.49 (s, 9H).

### Example 3

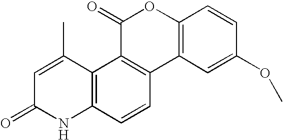

### 9-Methoxy-4-methyl-1H-6-oxa-1-aza-chrysene-2,5-dione (Compound 103, Structure 3 of Scheme II, where R²=methyl, R⁵=OCH₃, R⁶=H)

Compound 103 was synthesized in a similar fashion to that described in Example 1 as a white solid. ¹H NMR (500 MHz, DMSO-d₆) 12.12 (s, 1H), 8.59 (d, J=

9.0 Hz, 1H), 7.81 (d, J=9.0 Hz, 1H), 7.81 (d, J=2.9 Hz, 1H), 7.40 (d, J=9.0 Hz, 1H), 7.17 (dd, J=9.0, 2.9 Hz, 1H), 6.62 (m, 1H), 3.89 (s, 3H), 2.43 (d, J=0.8 Hz, 3H).

### Example 4

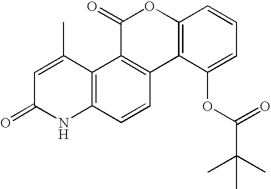

### 2,2-Dimethylpropionic acid 4-methyl-2,5-dioxo-2,5-dihydro-1H-6-oxa-1-aza-chrysen-10-yl ester (Compound 104, Structure 3 of Scheme VI, where R²=methyl. R⁵=H, R⁶=O—CO-t-Bu)

Compound 104 was synthesized in a similar fashion to that described in Example 1 as a white solid. ¹H NMR (500 MHz, CDCl₃) 12.74 (s, 1H), 8.63 (d, J=9.0 Hz, 1H), 7.77 (d, J=9.0 Hz, 1H), 7.49 (t, J=8.2 Hz, 1H), 7.33 (dd, J=8.2, 1.2 Hz, 1H), 6.95 (dd, J=8.2, 1.2 Hz, 1H), 6.83 (q, J=1.0 Hz, 1H), 2.61 (d, J=1.0 Hz, 3H), 1.49 (s, 9H).

### Example 5

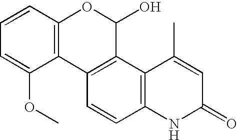

### (±)-5-Hydroxy-10-methoxy-4-methyl-1H,5H-6-oxa-1-aza-chrysen-2-one (Compound 105, Structure 4 of Scheme III, where R²=methyl, R⁵=H, R⁶=OCH₃)

A mixture of compound 3 in CH₂Cl₂ was cooled to 0° C. and treated with a 1.5 M toluene solution of diisobutylaluminum hydride (3 eq.) at 0° C. The reaction mixture was stirred at rt for 3 hrs. MeOH was added to quench the reaction and the mixture was concentrated. Chromatography of the crude mixture afforded Compound 105 as a white solid. ¹H NMR (500 MHz, DMSO-d₆) 11.75 (s, 1H), 8.47 (d, J=9.5 Hz, 1H), 7.39-7.35 (m, 2H), 7.22 (t, J=8.2 Hz, 1H), 6.98 (d, J=5.5 Hz, 1H), 6.80 (d, J=8.0 Hz, 1H), 6.69 (d, J=7.5 Hz, 1H), 6.43 (s, 1H), 3.90 (s, 3H), 2.76 (s, 1H).

### Example 6

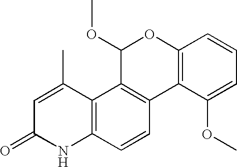

### (±)-5,10-Dimethoxy-4-methyl-1H,5H-6-oxa-1-aza-chrysen-2-one (Compound 106, Structure 5 of Scheme III, where R²=methyl, R⁵=H, R⁶=OCH₃)

To the mixture of compound 4 in MeOH was added p-toluene sulfonic acid monohydrate (1 eq.). The reaction mixture was stirred at rt for 30 min. and the mixture was concentrated. Chromatography of the crude mixture afforded Compound 106 as a white solid. ¹H NMR (500 MHz, DMSO-d₆) 11.81 (s, 1H), 8.48 (d, J=9.0 Hz, 1H), 7.44 (d, J=8.5 Hz, 1H), 7.28 (t, J=8.5 Hz, 1H), 6.86 (d, J=, 8.5 Hz, 1H), 6.83 (d, J=,8.5 Hz, 1H), 6.67 (s, 1H), 6.48 (s, 1H), 3.92 (s, 3H), 3.42 (s, 3H), 2.70 (s, 3H).

### Example 7

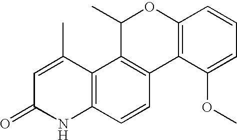

### (±)-10-Methoxy-4,5-dimethyl-1H,5H-6-oxa-1-aza-chrysen-2-one (Compound 107, Structure 6 of Scheme III, where R²=methyl, R⁵=H, R⁶=OCH₃)

To the mixture of compound 3 in THF was added methylmagnesium bromide (1.0 M solution in butyl ether) at rt. The reaction mixture was stirred at rt overnight and was concentrated. The crude product and triethylsilane in CH₂Cl₂ was cooled to −78° C. and treated with BF₃-OEt₂. The reaction mixture was then allowed to warm up to rt and the mixture was concentrated. Chromatography of the crude mixture afforded Compound 107 as a white solid. ¹H NMR (500 MHz, Acetone-d₆) 8.55 (d, J= 8.5 Hz, 1H), 7.42 (d, J=8.5 Hz, 1H), 7.22 (t, J=8.0 Hz, 1H), 6.79 (dd, J=8.0, 1.0 Hz, 1H), 6.63 (dd, J=8.0, 1.0 Hz, 1H), 6.44 (d, J=1.0 Hz, 1H), 6.40 (q, J=6.5 Hz, 1H), 3.97 (s, 3H), 2.78 (d, J=1.0 Hz, 1H), 1.46 (d, J=6.5 Hz, 3H).

### Example 8

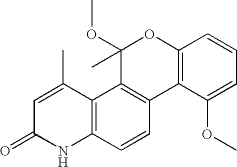

### (±)-5,10-Dimethoxy-4,5-dimethyl-1H,5H-6-oxa-1-azachrysen-2-one (Compound 108, Structure 7 of Scheme III, where R²=methyl, R⁵=H, R⁶=OCH₃)

Compound 108 was isolated from the reaction mixture of Compound 107 as a byproduct. ¹H NMR (500 MHz, Acetone-d₆) 10.63 (s, 1H), 8.35 (d, J=9.0 Hz, 1H), 7.44 (d, J=9.0 Hz, 1H), 7.25 (t, J=8.0 Hz, 1H), 6.83 (dd, J=8.0, 1.0 Hz, 1H), 6.71 (dd, J=7.5, 1.0 Hz, 1H), 6.45 (dd, J=2.5, 1.0 Hz, 1H), 3.95 (s, 3H), 3.33 (s, 3H), 2.70 (d, J=1.5 Hz, 3H), 1.87 (s, 3H).

### Example 9

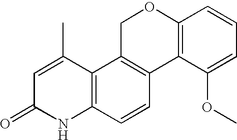

### 10-Methoxy-4-methyl-1H,5H-6-oxa-1-azachrysen-2-one (Compound 109, Structure 8of Scheme III, where R²=methyl, R⁵=H, R⁶=OCH₃)

A mixture of Compound 105 (Structure 4 of Scheme III, where R²=methyl, R⁵=H, R⁶=OCH₃) and triethylsilane in CH₂Cl₂ was cooled to −78° C. and treated with BF₃-OEt₂. The reaction mixture was then stirred at 0° C. for 30 min. and the mixture was concentrated. Chromatography of the crude mixture afforded Compound 109 as a white solid. ¹H NMR (500 MHz, DMSO-d₆) 11.72 (s, 1H), 8.27 (d, J=9.0 Hz, 1H), 7.32 (d, J=9.0 Hz, 1H), 7.21 (t, J=8.5 Hz, 1H), 6.81 (d, J=8.5 Hz, 1H), 6.66 (dd, J=8.5, 1.0 Hz, 1H), 6.40 (s, 1H), 5.50 (s, 2H), 3.88 (s, 3H), 2.57 (s, 3H).

### Example 10

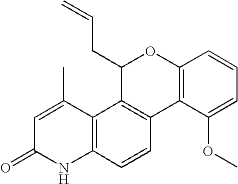

### (±)-5-Allyl-10-methoxy-4-methyl-1H,5H-6-oxa-1-aza-chrysen-2-one (Compound 110, Structure 9 of Scheme III, where R²=methyl, R⁵=H, R⁶=OCH₃)

A mixture of Compound 106 (Structure 5 of Scheme III, where R²=methyl, R⁵=H, R⁶=OCH₃) was treated with allyl trimethylsilane in a similar fashion as that described in Example 9 to afford Compound 110 as a white solid. ¹H NMR (500 MHz, CDCl₃) 11.16 (s, 1H), 8.58 (d, J=9.0 Hz, 1H), 7.33 (d, J=9.0 Hz, 1H), 7.21 (t, J=8.2 Hz, 1H), 6.68 (dd, J=8.2, 1.7 Hz, 2H), 6.59 (s, 1H), 6.24 (dd, J=10.7, 2.7 Hz, 1H), 5.95-5.88 (m, 1H), 5.14 (dd, J=10.5, 1.5 Hz, 1H), 5.08 (dd, J=17.0, 1.5 Hz, 1H), 3.96 (s, 3H), 2.82-2.77 (m, 1H), 2.76 (s, 3H), 2.30 (ddt, J=15.7, 7.2, 1.5 Hz, 1H).

### Example 11

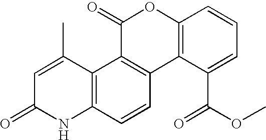

### 4-Methyl-2,5-dioxo-2,5-dihydro-1H-6-oxa-1-aza-chrysene-10-carboxylic acid methyl ester (Compound 111, Structure 14 of Scheme IV)

The solution of compound 10 in N,N-dimethylacetamide was kept at reflux for 17 hrs. After the reaction mixture was cooled down to rt, KOH and methyliodide was added. The reaction mixture was kept at rt for 4 hrs and the mixture was concentrated. Chromatography of the crude mixture afforded compound 11 as a white solid.

A mixture of compound 11 and 10% Pd/C in DMF was subject to hydrogenation (40 psi) in a Parr reactor for 2 hr. Reaction mixture was then filtered through celite. The mixture was then concentrated. DMF was then added. To the solution was added N-bromosuccinimide. The reaction mixture was kept at rt for 1.5 hrs. Water was added to the reaction mixture. Precipitate was filtered and dried to afford compound 12.

The mixture of compound 12 and 3-butenoic anhydride was stirred at rt for 3 hrs and the mixture was concentrated. Chromatography of the crude mixture afforded compound 13.

A mixture of compound 13, Pd(OAc)₂, (o-tolyl)₃P, and Et₃N in DMF was degassed and stirred at 110° C. for 24 hrs and the mixture was concentrated. Chromatography of the crude mixture afforded Compound 111 as a white solid. ¹H NMR (500 MHz, DMSO-d₆) 12.24 (s, 1H), 7.78 (d, J=9.0 Hz, 1H), 7.73 (d, J=8.5 Hz, 1H), 7.63-7.61 (m, 2H), 7.54 (dd, J=7.0, 2.0 Hz, 1H), 7.63 (d, J=1.0 Hz, 1H), 3.92 (s, 1H), 2.46 (d, J=1.0 Hz, 3H).

### Example 12

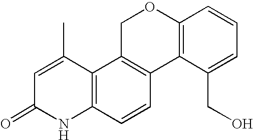

### 10-Hydroxymethyl-4-methyl-1H,5H-6-oxa-1-azachrysen-2-one (Compound 112, Structure 15 of Scheme IV)

A mixture of Compound 111 (Structure 14 of Scheme IV) in CH₂Cl₂ was cooled to 0° C. and treated with a 1.5 M toluene solution of diisobutylaluminum hydride at 0° C. The reaction mixture was stirred at rt for 3 hrs. MeOH was added to quench the reaction and the mixture was concentrated. The crude product, triethylsilane in CH₂Cl₂ was cooled to −78° C. and treated with BF₃-OEt₂. The reaction mixture was then stirred at 0° C. for 30 min. and the mixture was concentrated. Chromatography of the crude mixture afforded Compound 112 as a white solid. ¹H NMR (500 MHz, CDCl₃/CD₃OD) 8.13 (d, J=9.0 Hz, 2H), 7.43 (d, J=8.5 Hz, 1H), 7.29-7.28 (m, 2H), 7.02 (dd, J=6.5, 2.5 Hz, 1H), 6.56 (d, J=1.0 Hz, 1H), 5.49 (s, 2H), 5.40 (s, 1H), 4.77 (s, 2H), 2.67 (d, J=1.0 Hz, 3H).

### Example 13

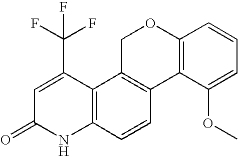

### 10-Hydroxymethyl-4-trifluoromethyl-1H,5H-6-oxa-1-aza-chrysen-2-one (Compound 113, Structure 8 of Scheme V, where R²=methyl, R⁵=H, R⁶=OCH₃)

To a mixture of compound 1,4-dimethylaminopyridine in THF was added trimethylacetyl chloride at rt. The reaction mixture was stirred at rt for 2 days and the mixture was concentrated. Chromatography of the crude mixture afforded compound 16 as a white solid.

A mixture of compound 16 in CH₂Cl₂ was cooled to −78° C. and treated with a 1.5 M CH₂Cl₂ solution of diisobutylaluminum hydride at −78° C. The reaction mixture was stirred at −78° C. for 1 hr. MeOH was added to quench the reaction and the mixture was concentrated. Chromatography of the crude mixture afforded compound 17 as a white solid.

A mixture of compound 17 and triethylsilane in CH₂Cl₂ was cooled to −78° C. and treated with BF₃-OEt₂. The reaction mixture was then stirred at 0° C. for 30 min. and the mixture was concentrated. Chromatography of the crude mixture afforded compound 18 as a white solid.

To a solution of compound 18 in dry THF was added n-BuLi (2.5 M in hexane) at −78° C. and kept for 30 min. Trifluoroacetate was then added at −78° C. The reaction mixture was stirred at −78° C. for 30 min. before warmed up to rt. The reaction mixture was stirred at rt for 2 hrs. and was concentrated. Chromatography of the crude mixture afforded compound 19 as a white solid.

A mixture of compound 19 and (ethoxycarbonylmethylene)triphenyl-phosphorane in dry toluene was stirred at reflux for 5 hrs. and the mixture was concentrated. Chromatography of the crude mixture afforded compound 20 as a white solid.

A mixture of compound 20, acetic acid and cone. HCl was heated at reflux for 16 hrs. After the reaction was cooled down to rt, the precipitate was filtered and washed with water. Chromatography of the crude mixture afforded Compound 113 as a yellow solid. ¹H NMR (500 MHz, DMSO-d₆) 12.51 (s, 1H), 8.41 (d, J=9.0 Hz, 1H), 7.50 (d, J=9.0 Hz, 1H), 7.27 (t, J=8.2 Hz, 1H), 7.16 (s, 1H), 6.85 (dd, J=8.5, 1.0 Hz, 1H), 6.70 (dd, J=8.0, 1.0 Hz, 1H), 5.14 (s, 2H), 3.90 (s, 3H).

### Example 14

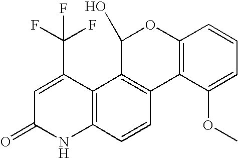

### (±)-5-Hydroxy-10-methoxy-4-trifluoromethyl-1H,5H-6-oxa-1-aza-chrysen-2-one (Compound 114, Structure 4 of Scheme III, where R²=trifluoromethyl, R⁵=H, R⁶=OCH₃)

A mixture of Compound 113 (Structure 8 of Scheme III, where R⁵═H, R⁶═OCH₃) and pyridinium chlorochromate in CH₂Cl₂ was heated in a sealed tube at 60° C. for 20 hrs. and the mixture was concentrated. Chromatography of the crude mixture afforded compound 114 as a yellow solid. ¹H NMR (500 MHz, DMSO-d₆) 12.52 (s, 1H), 8.70 (d, J=8.5 Hz, 1H), 7.55 (d, J=9.5 Hz, 1H), 7.27 (t, J=8.0 Hz, 1H), 7.22 (d, J=5.5 Hz, 1H), 7.17 (s, 1H), 6.83 (d, J=9.0 Hz, 1H), 7.78 (d, J=4.5 Hz, 1H), 8.72 (d, J=8.0 Hz, 1H), 3.94 (s, 3H).

### Example 15

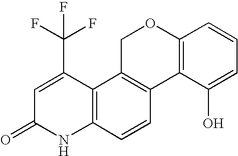

### 10-Hydroxy-4-trifluoromethyl-1H,5H-6-oxa-1-aza-chrysen-2-one (Compound 115, Structure 8 of Scheme V, where R²=trifluoromethyl, R⁵=H, R⁶=OH)

To a mixture of Compound 113 (Structure 8 of Scheme V, where R²=trifluoromethyl, R⁵=H, R⁶=OCH₃) in dry CH₂Cl₂ at −40° C. was added BBr₃. The reaction mixture was then stirred at rt for 1 hr. and the mixture was concentrated. Chromatography of the crude mixture afforded Compound 115 as a yellow solid. ¹H NMR (500 MHz, DMSO-d₆) 12.49 (d, J=1.8 Hz, 1H), 10.26 (s, 1H), 8.56 (d, J=8.9 Hz, 1H), 7.49 (d, J=8.9 Hz, 1H), 7.15 (d, J=1.8 Hz, 1H), 7.08 (t, J=8.1 Hz, 1H), 6.66 (dd, J=8.1, 1.1 Hz, 1H), 6.52 (dd, J=8.1, 1.1 Hz, 1H), 5.13 (s, 2H).

### Example 16

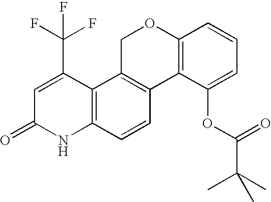

### 2,2-Dimethyl-propionic acid 2-oxo-4-trifluoromethyl-2,5-dihydro-1H-6-oxa-1-aza-chrysen-10-yl ester (Compound 116, Structure 8 of Scheme V, where R²=trifluoromethyl, R⁵=H, R⁶=O—CO-t-Bu)

To a mixture of Compound 115, 4-dimethylaminopyridine and triethylamine in DMF was added trimethylacetyl chloride at rt. The reaction mixture was stirred at rt for 17 hrs. and the mixture was concentrated. Chromatography of the crude mixture afforded Compound 116 as a yellow solid. ¹H NMR (500 MHz, CDCl₃) 11.92 (s, 1H), 8.18 (d, J=8.6 Hz, 1H), 7.43 (d, J=8.6 Hz, 1H), 7.31 (s, 1H), 7.28 (t, J=8.1 Hz, 1H), 6.98 (dd, J=8.1, 1.0 Hz, 1H), 6.79 (dd, J=8.1, 1.0 Hz, 1H), 5.33 (s, 2H), 1.36 (s, 9H).

### Example 17

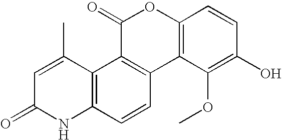

### 9-Hydroxy-10-methoxy-4-methyl-1H-6-oxa-1-aza-chrysene-2,5-dione (Compound 117, Structure 3 of Scheme II, where R²=methyl, R⁵=OH, R⁶=OCH₃)

A mixture of Compound 102 (Structure 3 of Scheme II, where R²=methyl, R⁵=O—CO-t-Bu, R⁶=OCH₃) in 6M HCl and 1,4-dioxane was heated at 80° C. for 20 hrs. After removal of 1,4-dioxane, the precipitate was filtered and washed with water.

The solid was recrystallized from MeOH to afford Compound 117 as a white solid. ¹H NMR (500 MHz, DMSO-d₆) 12.17 (s, 1H), 9.65 (s, 1H), 9.02 (d, J=9.3 Hz, 1H), 7.82 (d, J=9.3 Hz, 1H), 7.13 (d, J=8.9 Hz, 1H), 7.10 (d, J=8.9 Hz, 1H), 6.62 (q, J=1.0 Hz, 1H), 3.85 (s, 3H), 2.43 (d, J=1.0 Hz, 3H).

### Example 18

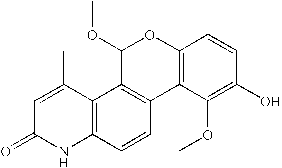

### (±)-9-Hydroxy-5,10-dimethoxy-4-methyl-1H,5H-6-oxa-1-aza-chrysen-2-one (Compound 118, Structure 5 of Scheme III, where R²=methyl, R⁵=OH, R⁶=OCH₃)

Compound 118 was synthesized from Compound 117 (Structure 3 of Scheme III, where R²=methyl,_R⁵=OH, R⁶=OCH₃) in a similar fashion as that described in Examples 5 and 6 as a white solid. ¹H NMR (500 MHz, DMSO-d₆) 11.85 (s, 1H), 9.05 (s, 1H), 8.45 (d, J=8.8 Hz, 1H), 7.47 (d, J=8.8 Hz, 1H), 6.83 (d, J=8.5 Hz, 1H), 6.79 (d, J=8.5 Hz, 1H), 6.58 (s, 1H), 6.48 (m, 1H), 3.67 (s, 3H), 3.42 (s, 3H), 2.70 (d, J=0.8 Hz, 3H)

### Example 19

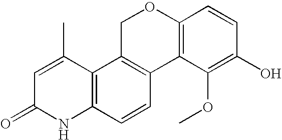

### 9-Hydroxy-10-methoxy-4-methyl-1H,5H-6-oxa-1-aza-chrysen-2-one (Compound 119, Structure 8 of Scheme III, where R²=methyl, R⁵=OH, R⁶=OCH₃)

Compound 119 was synthesized from Compound 102 (Structure 3 of Scheme III, where R²=methyl,_R⁵=OH, R⁶=OCH₃) in a similar fashion as that described in Examples 5 and 9 as a white solid. ¹H NMR (500 MHz, DMSO-d₆) 11.76 (s, 1H), 9.01 (s, 1H), 8.26 (d, J=8.8 Hz, 1H), 7.35 (d, J=8.8 Hz, 1H), 6.75 (d, J=8.6 Hz, 1H), 6.64 (d, J=8.6 Hz, 1H), 6.41 (m, 1H), 5.44 (s, 2H), 3.64 (s, 3H), 2.56 (d, J=0.8 Hz, 3H).

### Example 20

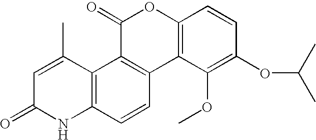

### 9-Isopropoxy-10-methoxy-4-methyl-1H-6-oxa-1-aza-chrysene-2,5-dione (Compound 120, Structure 3 of Scheme II, where R²=methyl, R⁵=OCH(CH₃)₂, R⁶=OCH₃)

To a mixture of Compound 117 (Structure 3 of Scheme II, where R²=methyl, R⁵=OH, R⁶=OCH₃) and NaHCO₃ in dry DMSO was added 2-iodopropane at rt. The reaction mixture was stirred at 50° C. for 24 hrs. Water was then added. The precipitate was filtered and washed with water. Chromatography of the crude mixture afforded Compound 120 as a white solid. ¹H NMR (500 MHz, DMSO-d₆) 12.15 (s, 1H), 9.00 (d, J=9.0 Hz, 1H), 7.80 (d, J=9.0 Hz, 1H), 7.32 (d, J=9.0 Hz, 1H), 7.18 (d, J=9.0 Hz, 1H), 6.60 (q, J=1.1 Hz, 1H), 4.66 (sept, J=6.1 Hz, 1H), 3.88 (s, 3H), 2.41 (d, J=1.1 Hz, 3H), 1.33 (d, J=6.1 Hz, 6H).

### Example 21

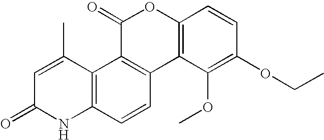

### 9-Ethoxy-10-methoxy-4-methyl-1H-6-oxa-1-aza-chrysene-2,5-dione (Compound 121. Structure 3 of Scheme II, where R²=methyl, R⁵=OCH₂CH₃, R⁶=OCH₃)

Compound 117 (Structure 3 of Scheme II, where R²=methyl, R⁵=OH, R⁶=OCH₃) was treated with NaHCO₃ and iodoethane in a similar fashion as that described in Example 20 to afford Compound 121 as a yellow solid. ¹H NMR (500 MHz, DMSO-d₆) 12.16 (s, 1H), 9.01 (d, J=9.0 Hz, 1H), 7.80 (d, J=9.0 Hz, 1H), 7.32 (d, J=9.0 Hz, 1H), 7.19 (d, J=9.0 Hz, 1H), 6.60 (q, J=1.1 Hz, 1H), 4.14 (q, J=7.0 Hz, 2H), 3.89 (s, 3H), 241 (d, J=1.1 Hz, 3H), 1.40 (t, J=7.0 Hz, 3H).

### Example 22

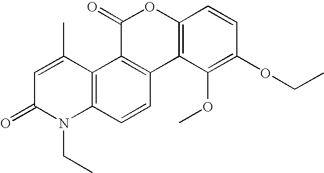

### 9-Ethoxy-1-ethyl-10-methoxy-4-methyl-1H-6-oxa-1-aza-chrysene-2,5-dione (Compound 122, Structure 3a of Scheme II, where R⁵=OCH₂CH₃, R⁶=OCH₃, R^{A}=CH₂CH₃)

Compound 122 was prepared by alkylation of Compound 121. ¹H NMR (500 MHz, DMSO-d₆) 9.08 (d, J=9.5 Hz, 1H), 8.16 (d, J=9.5 Hz, 1H), 7.34 (d, J=9.0 Hz, 1H), 7.21 (d, J=9.0 Hz, 1H), 6.70 (q, J=1.0 Hz, 1H), 4.38 (q, J=7.0 Hz, 2H), 4.16 (q, J=7.0 Hz, 2H), 3.92 (s, 3H), 2.39 (d, J=1.0 Hz, 3H), 1.41 (t, J=7.0 Hz, 3H), 1.26 (t, J=7.0 Hz, 3H).

### Example 23

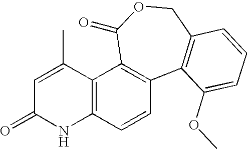

### 4-Methoxy-10-methyl-7,13-dihydro-12-oxa-7-aza-benzo[3,4]cyclohepta[1,2-α]-naphthalene-8,11-dione (Compound 123, Structure 32 of Scheme VI)

To 3-methoxybenzyl alcohol in hexane/ether was added 2.5M n-BuLi slowly at 0° C. It was warmed up to room temp and stirred at room temp for 2 hrs. B(OMe)₃ was added quickly. The reaction mixture was diluted with saturated NH₄Cl and stirred at rt for 15 min. 2M HCl was added to the reaction and extracted with EtO Ac. The EtOAc layer was extracted with 2M NaOH. NaOH solution was acidified by cone. HCl to pH4, and extracted with EtOAc. The organic layer was dried by Na₂SO₄ and concentrated to afford compound 27.

To compound 27 in DMF was added PdCl₂(dppf), K₃PO₄ and methyl 2-bromo-5-nitrobenzoate. The solution was stirred at rt. More PdCl₂(dppf) was added after 20 hrs. After another 20 hrs, the reaction was quenched by water and extracted with EtOAc. The EtOAc layer was dried by Na₂SO₄ and purified by chromatography to afford compound 28.

To compound 28 in DMF and water was added K₃PO₄. The solution was stirred at rt for 15 min. The reaction was quenched by water and extracted with EtOAc. The EtOAc layer was filtered through silica gel and dried with Na₂SO₄ and concentrated to afford compound 29.

Compound 29 was dissolved in EtOAc and hydrogenated under hydrogen pressure (50 psi) at room temp for 3 hrs. The reaction was filtered through silica gel, and washed by EtOAc. The EtOAc layer was dried by Na₂SO₄ and concentrated to afford the amine intermediate. The amine intermediate was dissolved in EtOAc and DMF. NBS was added very slowly in 1 hr. It was stirred at rt until GC-MS showed no starting material. The reaction was quenched by water and extracted with EtOAc. The EtOAc layer was filtered through silica gel and dried by Na₂SO₄ and concentrated to afford compound 30.

To compound 30 in CH₂Cl₂ was added 4-dimethylaminopyridine and vinyl acetyl chloride at rt. It was stirred at rt for 16 hrs. The reaction mixture was filtered through silica gel, and washed by CH₂Cl₂. The CH₂Cl₂ layer was dried by Na₂SO₄ and concentrated to afford compound 31.

To compound 31 in toluene in a sealed tube was added Pd(OAC)₂, (o-tolyl)₃P and Et₃N. It was stirred at 110° C. for 16 hrs. The reaction mixture was filtered through silica gel, and washed by EtOAc and MeOH. The organic layer was dried over Na₂SO₄ and purified by chromatography to afford Compound 123. ¹H NMR (500 MHz, CDCl₃) 10.76 (s, 1H), 7.98 (d, J=8.8 Hz, 1H), 7.42 (d, J=8.8 Hz, 1H), 7.40 (dd, J=8.2, 7.5 Hz, 1H), 7.13 (dd, J=7.5, 1.0 Hz, 1H), 7.11 (dd, J=8.2, 1.0 Hz, 1H), 6.67 (q, J=1.1 Hz, 1H), 5.08 (d, J=12.0 Hz, 1H), 5.03 (d, J=12.0 Hz, 1H), 3.87 (s, 3H), 2.54 (d, J=1.1 Hz, 3H).

### Example 24

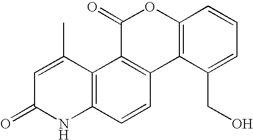

### 10-Hydroxymethyl-4-methyl-1H-6-oxa-1-aza-chrysene-2,5-dione (Compound 124, Structure 33 of Scheme VI)

To Compound 123 (Example 23) in CH₂Cl₂ was added BBr₃ at −78° C. It was warmed up to rt and stirred at rt for 16 hrs. The reaction was quenched by water and extracted with EtOAc. The EtOAc layer was dried with Na₂SO₄ and purified by chromatography to afford Compound 124. ¹H NMR (500 MHz, CDCl₃) 12.02 (s, 1H), 8.59 (d, J=8.9 Hz, 1H), 7.88 (d, J=8.9 Hz, 1H), 7.49 (t, J=7.8 Hz, 1H), 7.42 (dd, J= 7.8, 1.4 Hz, 1H), 7.41 (dd, J=7.8, 1.4 Hz, 1H), 6.82 (q, J=1.0 Hz, 1H), 4.84 (s, 2H), 2.62 (d, J=1.0 Hz, 3H).

### Example 25

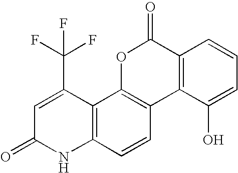

### 10-Hydroxy-4-trifluoromethyl-1H-5-oxa-1-azachrysene-2,6-dione (Compound 125. Structure 39 of Scheme VIII, where R⁵=H, R⁶=OH)

To a mixture of compound 34 and NaHCO₃ in dry DMF was added iodo-methane at rt. The reaction mixture was stirred at rt for 16 hrs. and was concentrated. Chromatography of the crude mixture afforded compound 35 as a white solid.

To a mixture of compound 35 and cesium fluoride in dry DMF was added 2-iodopropane at rt. The reaction mixture was stirred at rt for 17 hrs. and concentrated. Chromatography of the crude mixture afforded compound 36 as a white solid.

The mixture of compound 36, compound 37, Pd(PPh₃)₄ in 1,4-dioxane and 2M Na₂CO₃ was degassed and heated at 110° C. for 41 hrs. and the mixture was concentrated. Chromatography of the crude mixture afforded compound 38 as a white solid.

Compound 38 was stirred at reflux in 48% HBr and acetic acid overnight. The reaction mixture was then poured into ice. Precipitate was filtered and washed with water. Chromatography of the crude mixture afforded Compound 125 as a yellow solid. ¹H NMR (500 MHz, DMSO-d₆) 9.50 (d, J=9.2 Hz, 1H), 7.73 (dd, J=7.6, 1.0 Hz, 1H), 7.49 (dd, J=7.6, 8.0 Hz, 1H), 7.41 (dd, J=8.0, 1.0 Hz, 1H), 7.40 (d, J=9.2 Hz, 1H), 7.12 (s, 1H).

### Example 26

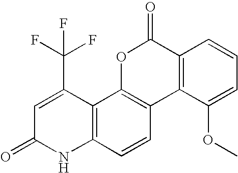

### 10-Methoxy-4-trifluoromethyl-1H-5-oxa-1-azachrysene-2,6-dione (Compound 126, Structure 39 of Scheme VIII, where R⁵=H, R⁶=OCH₃)

Compound 126 was isolated from the reaction mixture of Compound 125 (Example 25) as a second product. ¹H NMR (500 MHz, DMSO-d₆) 9.09 (d, J=9.4 Hz, 1H), 7.91 (dd, J=5.6, 3.4 Hz, 1H), 7.62 (d, J=3.4 Hz, 1H), 7.62 (d, J=5.6 Hz, 1H), 7.30 (d, J=9.4 Hz, 1H), 6.95 (s, 1H), 4.08 (s, 3H).

### Example 27

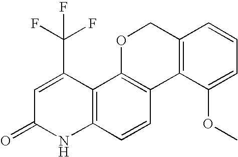

### 10-Methoxy-4-trifluoromethyl-1,6-dihydro-5-oxa-1-azachrysen-2-one (Compound 127, Structure 41 of Scheme VIII, where R⁵=H, R⁶=OCH₃)

A mixture of Compound 126 (Structure 39 of Scheme VIII, where R⁵=H, R⁶=OCH₃) in CH₂Cl₂ was cooled to 0° C. and treated with a 1.5 M toluene solution of diisobutylaluminum hydride at 0° C. The reaction mixture was stirred at rt for 3 hrs. MeOH was added to quench the reaction and the mixture was concentrated to afford compound 40. The crude product 40, triethylsilane in CH₂Cl₂ was cooled to −78° C. and treated with BF₃-OEt₂. The reaction mixture was then stirred at 0° C. for 30 min. and the mixture was concentrated. Chromatography of the crude mixture afforded Compound 127 as a yellow solid. ¹H NMR (500 MHz, DMSO-d₆) 12.39 (s, 1H), 8.54 (d, J=8.8 Hz, 1H), 7.34 (dd, J=8.4, 7.3 Hz, 1H), 7.14 (d, J=8.8 Hz, 1H), 7.13 (dd, J=8.4, 0.9 Hz, 1H), 6.97 (s, 1H), 6.96 (dd, J=7.3, 0.9 Hz, 1H), 5.07 (s, 2H), 3.91 (s, 3H).

### Example 28

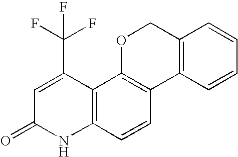

### 4-Trifluoromethyl-1,6-dihydro-5-oxa-1-aza-chrysen-2-one (Compound 128. Structure 41 of Scheme VIII, where R⁵=H, R⁶=H)

Compound 128 was synthesized in a similar fashion to that described in Example 27 as a yellow solid. ¹H NMR (500 MHz, Acetone-d₆) 11.18 (s, 1H), 8.15 (d, J=8.5 Hz, 1H), 7.84 (dd, J=7.7, 1.0 Hz, 1H), 7.45 (ddd, J=7.7, 7.0, 1.0 Hz, 1H), 7.37 (td, J=7.7, 1.0 Hz, 1H), 7.34 (dd, J=7.0, 1.0 Hz, 1H), 7.27 (d, J=8.5 Hz, 1H), 6.98 (m, 1H), 5.26 (s, 2H).

### Example 29

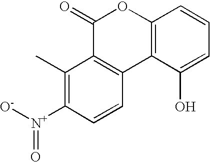

### 1-Hydroxy-7-methyl-8-nitrobenzo[c]coumarin (Compound 129, Structure 45 of Scheme IX, where R⁵=H, R⁶=OH, R⁷=methyl, R⁸=nitro)

A mixture of compound 42, 2,6-dimethoxyphenylboronic acid 43, Pd(PPh₃)₄, and 2M Na₂CO₃ (aq) in 1,4-dioxane was degassed and heated at 110° C. overnight. The mixture was concentrated. Chromatography of the crude mixture afforded Compound 44 as an oil.

To a solution of Compound 44 at −40° C. was added BBr₃ slowly. The reaction mixture was then allowed to warm to 0° C. After 10 min., the reaction mixture was warmed to rt and kept at rt for 1 hr. MeOH was added slowly at rt. Solid was formed, filtered and washed with H₂O and hexanes to afford Compound 129 as white solid. ¹H NMR (300 MHz, DMSO-d₆) 11.38 (s, 1H), 9.27 (dd, J=9.1, 0.5 Hz, 1H), 8.26 (d, J=9.1 Hz, 1H), 7.43 (t, J=8.2 Hz, 1H), 6.93 (dd, J=8.2, 1.1 Hz, 1H), 6.88 (dd, J=8.2, 1.1 Hz, 1H), 2.74 (s, 3H).

### Example 30

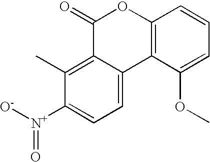

### 1-Methoxy-7-methyl-8-nitrobenzo[c]coumarin (Compound 130, Structure 45 of Scheme IX, where R⁵=H, R⁶=OMe, R⁷=methyl, R⁸=nitro)

To a solution of Compound 129 in dry DMF at rt was added NaH. After 30 min., CH₃I was added slowly at rt. The reaction mixture was then stirred at rt overnight. H₂O was added to quench the reaction. Solid was formed, filtered and washed with H₂O and dried. Crude product was purified by recrystallization from MeOH to afford Compound 130 as white solid. ¹H NMR (500 MHz, DMSO-d₆) 9.13 (dq, J=9.1, 0.5 Hz, 1H), 8.25 (d, J=9.1 Hz, 1H), 7.60 (t, J=8.4 Hz, 1H), 7.13 (dd, J=8.4, 1.0 Hz, 1H), 7.05 (dd, J=8.4, 1.0 Hz, 1H), 4.06 (s, 3H), 2.74 (s, 3H).

### Example 31

### IR Binding Assay

COS-1 cells in 96-well microtiter plates containing DMEM-10% FBS were transfected using standard techniques with plasmids pRShAR (2 ng/well), pRS-β-Gal (50 ng/well) and pGEM (48 ng/well). Six hours after transfection, media was removed, the cells were washed with PBS and fresh media was added. Twenty-four hours later, the media was changed to serum free DMEM and the cells were incubated for twenty-four hours.

After 24 hours in serum-free media, binding affinity of test compounds was determined by adding media containing 1 nM ³H-DHT and a test compound in concentrations ranging from 10⁻¹⁰ to 10⁻⁶ M or no test compound to separate wells of test plates. Three replicates were used for each sample. To determine the K_{d} for tritiated dihydrotestosterone (³H-DHT), media (DMEM-0.2% CA-FBS) containing ³H-DHT (in concentrations ranging from 12 nM to 0.24 nM) in the absence (total binding) or presence (non-specific binding) of a 100-fold molar excess of unlabeled DHT was added to separate wells of control plates.

After three hours at 37° C., aliquots of media from each well of both test plates and control plates were removed to estimate the amount of free ³H-DHT. The remaining media was removed, the cells were washed three times with PBS, and cells were lysed with a Triton X-100-based buffer. The lysates were assayed for amount of bound ³H-DHT and β-Gal activity using a scintillation counter and spectrophotometer, respectively.

### Analysis

Determination of K_{d} for ³H-DHT. Total binding was defined as binding of ³H-DHT in the absence of 100 molar excess of unlabeled DHT in the control plate. Specific binding was defined as binding of ³H-DHT in the presence of 100 molar excess of unlabeled DHT in the control plate. Specific binding was defined as the difference between the total binding and the nonspecific binding, normalized by the β-Gal rate. Specific binding was used in Scatchard analysis to determine the K_{d} for ³H-DHT. See, e.g., D. Rodbard, “Mathematics and statistics of ligand assays: an illustrated guide” In: J. Langon and J. J. Clapp, eds., Ligand Assay, Masson Publishing U.S.A., Inc., New York, pp. 45-99, (1981), the disclosure of which is herein incorporated by reference.

Test Compounds. To determine binding affinity (Kᵢ) for test compounds, the data from each concentration of test compound was calculated as a percentage of the signal for the sample with no test compound. The concentration of test compound that inhibited by 50% of the amount of ³H-DHT bound in the absence of test compound was identified graphically (IC₅₀) after log-logit transformation. The Kᵢ values were determined by the Cheng-Prusoff equation, where:

$$\frac{{IC}_{50}}{{K}_{i}} = ( 1 + {[}^{3} H \text{-} D H T ] ) / {K}_{d} {for }^{3} H \text{-} D H T$$

see e.g., Cheng, Y. C. and Prusoff, W. H. Biochem. Pharmacol. 22:3099 (1973). Kᵢ value ranges for certain AR binding compounds are shown in Table 1.

TABLE 1
Binding data

| Compound number | Example number | Binding to hAR |
|---|---|---|
| 101 | 1 | nd |
| 102 | 2 | B |
| 103 | 3 | C |
| 104 | 4 | A |
| 105 | 5 | C |
| 106 | 6 | nd |
| 107 | 7 | C |
| 108 | 8 | C |
| 109 | 9 | C |
| 110 | 10 | nd |
| 111 | 11 | C |
| 112 | 12 | B |
| 113 | 13 | B |
| 114 | 14 | C |
| 115 | 15 | A |
| 116 | 16 | A |
| 117 | 17 | A |
| 118 | 18 | C |
| 119 | 19 | A |
| 120 | 20 | B |
| 121 | 21 | C |
| 122 | 22 | C |
| 123 | 23 | C |
| 124 | 24 | B |
| 125 | 25 | A |
| 126 | 26 | B |
| 127 | 27 | B |
| 128 | 28 | A |
| 129 | 29 | A |
| 130 | 30 | C |

A = <10 nM, B = 10-50 nM, C = >50 nM.

Since modifications will be apparent to those of skill in this art, it is intended that this invention be limited only by the scope of the appended claims.

1. A compound of Formula I or Formula II: 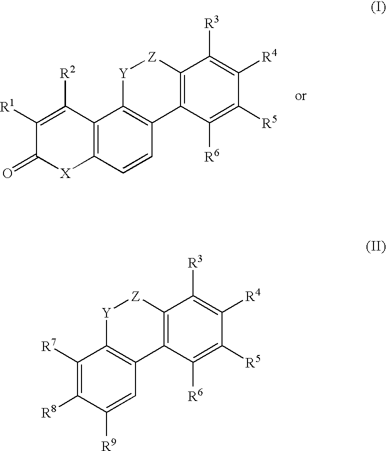
or a pharmaceutically acceptable salt or prodrug thereof, wherein:
R¹ is selected from among hydrogen, F, Cl, C₁-C₄ alkyl and C₁-C₄ haloalkyl;
R² is selected from among hydrogen, halogen, C₁-C₆ alkyl, C₁-C₆ haloalkyl, C₁-C₆ heteroalkyl and C₁-C₄ haloheteroalkyl;
R³ and R⁴ each independently is selected from among hydrogen, halogen, C₁-C₆ alkyl, C₁-C₆ haloalkyl, C₁-C₆ heteroalkyl and C₁-C₆ haloheteroalkyl;
R⁵ and R⁶ each independently is selected from among hydrogen, halogen, OR^{A}, S(O)ₙR^{A}, NR^{A}R^{B}, NR^{A}S(O)ₙR^{B}, COR^{A}, CO₂R^{A}, OC(O)R^{A}, CH₂OR^{A}, CONR^{A}R^{B}, an optionally substituted C₁-C₆ alkyl, an optionally substituted C₁-C₆ haloalkyl and an optionally substituted C₁-C₆ heteroalkyl; or R⁵ and R⁶ are linked to form a heterocyclic ring;
R⁷ and R⁹ each independently is selected from among hydrogen, halogen, OR^{A}, C₁-C₆ alkyl, C₁-C₆ haloalkyl, C₁-C₆ heteroalkyl and C₁-C₆ haloheteroalkyl;
R⁸ is selected from among NO₂, CN, COR^{A}, CO₂R^{A}, CONR^{A}R^{B}, SOR^{A}, SO₂R^{A}, SO₂NR^{A}R^{B}, C₁-C₆ haloalkyl and C₁-C₄ haloheteroalkyl;
R^{A} and R^{B} each independently is selected from among hydrogen, C₁-C₆ alkyl, C₁-C₆ haloalkyl and C₁-C₆ heteroalkyl; or R^{A} and R^{B} are linked to form a ring;
X is selected from among O, S and NR^{A};
Y and Z each independently is selected from among CR^{A}R^{B}, CR^{A}OR^{B}, CO, OCH₂, CH₂O, O, S(O)ₙ and NR^{A}, wherein, in formula I, Y and Z can be the same or different, and in formula II, Y and Z are different; and
n is selected from among 0, 1 and 2;
provided that, if R⁸ is NO₂, CN, COR^{A}, CO₂R^{A} or CONR^{A}R^{B}, and R⁹ is hydrogen, then R⁷ is not hydrogen; and
provided that, if R⁸ is CN or CO₂R^{A}, and R⁷ is methyl, then one of R⁵, R⁶ or R⁹ is not hydrogen.

2. The compound of claim 1 of Formula I, or a pharmaceutically acceptable salt or prodrug thereof.

3. A compound of claim 1, wherein:
R⁵ and R⁶ each independently is selected from among hydrogen, halogen, OR^{A}, COR^{A}, CO₂R^{A}, OC(O)R^{A}, CH₂OR^{A}, CONR^{A}R^{B}, an optionally substituted C₁-C₆ alkyl, an optionally substituted C₁-C₆ haloalkyl and an optionally substituted C₁-C₆ heteroalkyl.

4. The compound of claim 1, wherein:
R² is selected from among hydrogen, halogen, C₁-C₆ alkyl, C₁-C₆ haloalkyl, C₁-C₆ heteroalkyl and C₁-C₄ haloheteroalkyl;
R³ and R⁴ each independently is selected from among hydrogen, halogen, C₁-C₆ alkyl, C₁-C₆ haloalkyl, C₁-C₆ heteroalkyl and C₁-C₆ haloheteroalkyl; and
Y and Z each independently is selected from among CR^{A}R^{B}, CR^{A}OR^{B}, CO, OCH₂, CH₂O and O.

5. The compound of claim 1, wherein:
R⁵ and R⁶ each independently is selected from among hydrogen, halogen, OR^{A}, COR^{A}, CO₂R^{A}, OC(O)R^{A}, CH₂OR^{A}, CONR^{A}R^{B}, an optionally substituted C₁-C₆ alkyl, an optionally substituted C₁-C₆ haloalkyl and an optionally substituted C₁-C₆ heteroalkyl; and
X is NR^{A}.

6. The compound of claim 1, wherein:
R¹ is selected from among hydrogen, C₁-C₄ alkyl and C₁-C₄ haloalkyl;
R² is selected from among hydrogen, halogen, C₁-C₆ alkyl and C₁-C₆ haloalkyl;
R³ and R⁴ each independently is selected from among hydrogen, halogen, C₁-C₆ alkyl and C₁-C₆ haloalkyl;
R⁵ and R⁶ each independently is selected from among hydrogen, halogen, OR^{A}, COR^{A}, CO₂R^{A}, OC(O)R^{A}, CH₂OR^{A}, an optionally substituted C₁-C₆ alkyl and an optionally substituted C₁-C₆ haloalkyl; and
R^{A} and R^{B} each independently is selected from among hydrogen, C₁-C₆ alkyl and C₁-C₆ haloalkyl.

7. The compound of claim 1, wherein:
R¹ is selected from among hydrogen, C₁-C₄ alkyl and C₁-C₄ haloalkyl;
R² is selected from among hydrogen, halogen, C₁-C₆ alkyl and C₁-C₆ haloalkyl;
R³ and R⁴ each independently is selected from among hydrogen, halogen, C₁-C₆ alkyl and C₁-C₆ haloalkyl;
R⁵ and R⁶ each independently is selected from among hydrogen, halogen, OR^{A}, COR^{A}, CO₂R^{A}, OC(O)R^{A}, CH₂OR^{A}, an optionally substituted C₁-C₆ alkyl and an optionally substituted C₁-C₆ haloalkyl;
R^{A} and R^{B} each independently is selected from among hydrogen, C₁-C₆ alkyl and C₁-C₆ haloalkyl;
X is NR^{A}; and
Y and Z each independently is selected from among CR^{A}R^{B}, CR^{A}OR^{B}, CO, OCH₂, CH₂O and O.

8. The compound of claim 1, wherein:
R¹ is hydrogen;
R² is selected from among C₁-C₆ alkyl and C₁-C₆ haloalkyl;
R³ and R⁴ are hydrogen;
R⁵ and R⁶ each independently is selected from among hydrogen, halogen, OR^{A}, COR^{A}, CO₂R^{A}, OC(O)R^{A} and CH₂OR^{A};
R^{A} and R^{B} each independently is selected from among hydrogen and C₁-C₆ alkyl;
X is NR^{A}; and
Y and Z each independently is selected from among CR^{A}R^{B}, CR^{A}OR^{B}, CO, OCH₂, CH₂O and O.

9. The compound of claim 1, wherein:
R¹ is hydrogen;
R² is selected from among C₁-C₆ alkyl and C₁-C₆ haloalkyl;
R³ and R⁴ are hydrogen;
R⁵ and R⁶ each independently is selected from among hydrogen, halogen, OR^{A}, OC(O)R^{A} and CH₂OR^{A};
R^{A} and R^{B} each independently is selected from among hydrogen and C₁-C₆ haloalkyl;
X is NR^{A}; and
Y and Z each independently is selected from among CR^{A}R^{B}, CO and O.

10. The compound of claim 1, wherein:
R¹ is hydrogen;
R² is selected from among C₁-C₆ alkyl and C₁-C₆ haloalkyl;
R³ and R⁴ are hydrogen;
R⁵ and R⁶ each independently is selected from among hydrogen, halogen, OR^{A} and OC(O)R^{A};
R^{A} and R^{B} each independently is selected from among hydrogen and C₁-C₆ alkyl;
X is NR^{A}; and
Y and Z each independently is selected from among CR^{A}R^{B}, CO and O.

11. The compound of claim 1, wherein:
R¹ is selected from among hydrogen, C₁-C₄ alkyl and C₁-C₄ haloalkyl; and
R² is selected from among halogen, C₁-C₆ alkyl and C₁-C₆ haloalkyl.

12. The compound of claim 1, wherein:
R³ and R⁴ each independently is selected from among hydrogen, halogen, C₁-C₆ alkyl and C₁-C₆ haloalkyl; and
R⁵ and R⁶ each independently is selected from among hydrogen, halogen, OR^{A}, COR^{A}, CO₂R^{A}, OC(O)R^{A}, CH₂OR^{A}, C₁-C₆ alkyl, C₁-C₆ haloalkyl and C₁-C₆ heteroalkyl.

13. The compound of claim 1, wherein R⁵ and R⁶ each independently is selected from among hydrogen, halogen, OR^{A}, COR^{A}, CO₂R^{A}, OC(O)R^{A} and CH₂OR^{A}.

14. The compound of claim 1, wherein R⁵ and R⁶ each independently is selected from among hydrogen, F, hydroxy, methoxy, ethoxy, isopropoxy, hydroxymethyl, OC(O)^{t}Bu and CO₂Me.

15. The compound of claim 1, wherein:
R^{A} and R^{B} each independently is selected from among hydrogen and C₁-C₆ alkyl; and
Y and Z each independently is selected from among CR^{A}R^{B}, CR^{A}OR^{B}, CO, OCH₂, CH₂O and O.

16. The compound of claim 1, wherein
Y and Z each independently is selected from among CR^{A}R^{B}, CR^{A}OR^{B}, CO and O.

17. The compound of claim 1, wherein Y and Z each independently is selected from among CR^{A}OR^{B}, CO and O.

18. The compound of claim 1, wherein:
if X is NH, and Y is CO, and Z is O, and each of R¹, R³, R⁴, and R⁵ is hydrogen, and R² is CH₃, then R⁶ is not OCH₃.

19. The compound of claim 1, of Formula II, or a pharmaceutically acceptable salt or prodrug thereof.

20. The compound of claim 1, wherein R⁵ and R⁶ each independently is selected from among hydrogen, halogen, OR^{A}, COR^{A}, CO₂R^{A}, OC(O)R^{A}, CH₂OR^{A}, CONR^{A}R^{B}, an optionally substituted C₁-C₆ alkyl, an optionally substituted C₁-C₆ haloalkyl and an optionally substituted C₁-C₆ heteroalkyl.

21. The compound of claim 1, wherein:
R⁷ and R⁹ each independently is selected from among hydrogen, halogen, OR^{A}, C₁-C₆ alkyl and C₁-C₆ haloalkyl; and
R⁸ is selected from among NO₂, CN, COR^{A}, CO₂R^{A}, CONR^{A}R^{B}, SOR^{A}, SO₂R^{A} and SO₂NR^{A}R^{B}.

22. The compound of claim 1, wherein R⁸ is selected from among NO₂, SOR^{A}, SO₂R^{A} and SO₂NR^{A}R^{B}.

23. The compound of claim 1, wherein:
R⁷ and R⁹ each independently is selected from among hydrogen, C₁-C₆ alkyl and C₁-C₆ haloalkyl; and
R⁸ is NO₂.

24. The compound of claim 1, wherein:
R³ and R⁴ each independently is selected from among hydrogen, halogen, C₁-C₆ alkyl and C₁-C₆ haloalkyl; and
R⁷ and R⁹ each independently is selected from among hydrogen, C₁-C₆ alkyl and C₁-C₆ haloalkyl.

25. The compound of claim 1, wherein:
R⁵ and R⁶ each independently is selected from among hydrogen, halogen, OR^{A}, COR^{A}, CO₂R^{A}, OC(O)R^{A}, CH₂OR^{A}, CONR^{A}R^{B}, an optionally substituted C₁-C₆ alkyl and an optionally substituted C₁-C₆ haloalkyl;
R⁷ and R⁹ each independently is selected from among hydrogen, C₁-C₆ alkyl and C₁-C₆ haloalkyl; and
Y and Z each independently is selected from among CR^{A}R^{B}, CR^{A}OR^{B}, CO, OCH₂, CH₂O, O and NR^{A}.

26. The compound of claim 1, wherein:
R^{A} and R^{B} each independently is selected from among hydrogen, C₁-C₆ alkyl and C₁-C₆ haloalkyl; and
Y and Z each independently is selected from among CR^{A}R^{B}, CR^{A}OR^{B}, CO and O.

27. The compound of claim 1, wherein:
R³ and R⁴ are hydrogen;
R⁷ and R⁹ each independently is selected from among hydrogen, C₁-C₆ alkyl and C₁-C₆ haloalkyl;
R^{A} and R^{B} each independently is selected from among hydrogen, C₁-C₆ alkyl and C₁-C₆ haloalkyl; and
Y and Z each independently is selected from among CR^{A}R^{B}, CR^{A}OR^{B}, CO and O.

28. The compound of claim 1, wherein:
R³ and R⁴ are hydrogen;
R⁵ and R⁶ each independently is selected from among hydrogen, OR^{A}, COR^{A}, CO₂R^{A}, OC(O)R^{A}, CH₂OR^{A}, CONR^{A}R^{B}, an optionally substituted C₁-C₆ alkyl and an optionally substituted C₁-C₆ haloalkyl;
R⁷ and R⁹ each independently is selected from among hydrogen and C₁-C₆ alkyl;
R⁸ is NO₂;
R^{A} and R^{B} each independently is selected from among hydrogen, C₁-C₆ alkyl and C₁-C₆ haloalkyl; and
Y and Z each independently is selected from among CO and O.

29. The A compound of claim 1, wherein:
R³ and R⁴ are hydrogen;
R⁵ and R⁶ each independently is selected from among hydrogen, OR^{A} and OC(O)R^{A};
R⁷ and R⁹ each independently is selected from among hydrogen and C₁-C₆ alkyl;
R⁸ is NO₂;
R^{A} and R^{B} each independently is selected from among hydrogen, C₁-C₆ alkyl and C₁-C₆ haloalkyl; and
Y and Z each independently is selected from among CO and O.

30. A compound of Formula I or Formula II: 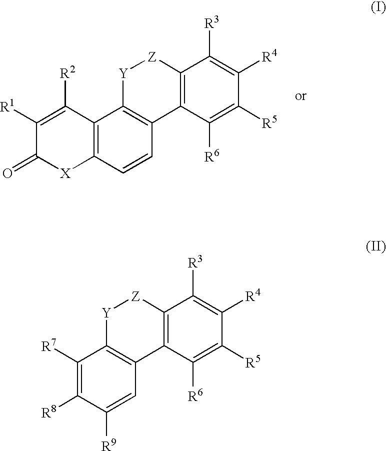
or a pharmaceutically acceptable salt or prodrug thereof, wherein:
R¹ is selected from among hydrogen, F, Cl, C₁-C₄ alkyl and C₁-C₄ haloalkyl;
R² is selected from among hydrogen, halogen, C₁-C₆ alkyl, C₁-C₆ haloalkyl, C₁-C₆ heteroalkyl and C₁-C₄ haloheteroalkyl;
R³ and R⁴ each independently is selected from among hydrogen, halogen, C₁-C₆ alkyl, C₁-C₆ haloalkyl, C₁-C₆ heteroalkyl and C₁-C₆ haloheteroalkyl;
R⁵ and R⁶ each independently is selected from among hydrogen, halogen, OR^{A}, S(O)ₙR^{A}, NR^{A}R^{B}, NR^{A}S(O)ₙR^{B}, COR^{A}, CO₂R^{A}, OC(O)R^{A}, CH₂OR^{A}, CONR^{A}R^{B}, an optionally substituted C₁-C₆ alkyl, an optionally substituted C₁-C₆ haloalkyl and an optionally substituted C₁-C₆ heteroalkyl; or R⁵ and R⁶ are linked to form a heterocyclic ring;
R⁷ and R⁹ each independently is selected from among hydrogen, halogen, OR^{A}, C₁-C₆ alkyl, C₁-C₆ haloalkyl, C₁-C₆ heteroalkyl and C₁-C₆ haloheteroalkyl;
R⁸ is selected from among NO₂, CN, COR^{A}, CO₂R^{A}, CONR^{A}R^{B}, SOR^{A}, SO₂R^{A}, SO₂NR^{A}R^{B}, C₁-C₆ haloalkyl and C₁-C₄ haloheteroalkyl;
R^{A} and R^{B} each independently is selected from among hydrogen, C₁-C₆ alkyl, C₁-C₆ haloalkyl and C₁-C₆ heteroalkyl; or R^{A} and R^{B} are linked to form a ring;
X is selected from among O, S and NR^{A};
Y and Z each independently is selected from among CR^{A}R^{B}, CR^{A}OR^{B}, CO, OCH₂, CH₂O, O, S(O)ₙ and NR^{A}; and
n is selected from among 0, 1 and 2;
provided that, if the compound has a structure of Formula II and if Y is the same as Z, then R⁴ is not isopropyl;
provided that, if the compound has a structure of Formula II, then at least one of R³, R⁴, R⁵, R⁶, and R⁹ is not hydrogen; and
provided that, if R⁸ is NO₂ and Y is CO and Z is O and R⁷ is hydrogen, then R⁹ is not hydrogen; and
provided that, if R⁸ is CO₂R^{A} or CONR^{A}R^{B}, then R⁴ is not F or R⁹ is not methoxy.

31. The compound of claim 30, wherein:
if X is NH, and Y is CO, and Z is O, and each of R¹, R³, R⁴, and R⁵ is hydrogen, and R² is CH₃, then R⁶ is not OCH₃.

32. The compound of claim 30 of Formula II, or a pharmaceutically acceptable salt or prodrug thereof.

33. The compound of claim 32, wherein R⁵ and R⁶ each independently is selected from among hydrogen, halogen, OR^{A}, COR^{A}, CO₂R^{A}, OC(O)R^{A}, CH₂OR^{A}, CONR^{A}R^{B}, an optionally substituted C₁-C₆ alkyl, an optionally substituted C₁-C₆ haloalkyl and an optionally substituted C₁-C₆ heteroalkyl.

34. The compound of claim 32 wherein:
R³ and R⁴ each independently is selected from among hydrogen, halogen, C₁-C₆ alkyl and C₁-C₆ haloalkyl;
R⁷ and R⁹ each independently is selected from among hydrogen, halogen, OR^{A}, C₁-C₆ alkyl, C₁-C₆ haloalkyl, C₁-C₆ heteroalkyl and C₁-C₆ haloheteroalkyl; and
R⁸ is selected from among NO₂, CN, COR^{A}, CO₂R^{A} and CONR^{A}R^{B}.

35. The compound of claim 32, wherein Y and Z each independently is selected from among CR^{A}R^{B}, CR^{A}OR^{B}, CO, OCH₂, CH₂O and O.

36. The compound of claim 35, wherein:
R³ and R⁴ are hydrogen;
R⁵ and R⁶ each independently is selected from among hydrogen, halogen, OR^{A}, COR^{A}, CO₂R^{A}, OC(O)R^{A}, CH₂OR^{A}, CONR^{A}R^{B}, an optionally substituted C₁-C₆ alkyl, an optionally substituted C₁-C₆ haloalkyl and an optionally substituted C₁-C₆ heteroalkyl;
R⁷ and R⁹ each independently is selected from among hydrogen, halogen, OR^{A}, C₁-C₆ alkyl, C₁-C₆ haloalkyl, C₁-C₆ heteroalkyl and C₁-C₆ haloheteroalkyl;
R⁸ is NO₂;
R^{A} and R^{B} each independently is selected from among hydrogen, C₁-C₆ alkyl and C₁-C₆ haloalkyl; and
Y and Z each independently is selected from among CR^{A}R^{B}, CR^{A}OR^{B}, CO, OCH₂, CH₂O and O.

37. The compound of claim 32 of formula IV: 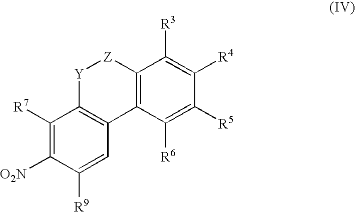
provided that, if Y is the same as Z, then R⁴ is not isopropyl; and
provided that at least one of R³, R⁴, R⁵, R⁶ and R⁹ is not hydrogen; and
provided that, if Y is CO and Z is O and R⁷ is hydrogen, then R⁹ is not hydrogen.

38. The compound of claim 37, wherein Y and Z each independently is selected from among CR^{A}R^{B}, CR^{A}OR^{B}, CO, OCH₂, CH₂O and O.

39. The compound of claim 38, wherein Y and Z each independently IS selected from among CO and O.

40. The compound of claim 32 of formula V: 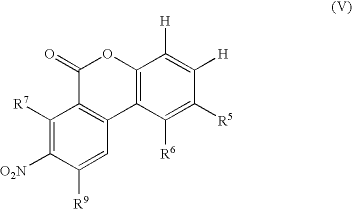
provided that R⁷ and R⁹ are not hydrogen at the same time.

41. The A compound of claim 40, wherein R⁵ and R⁶ each independently is selected from among hydrogen, halogen, OR^{A} and OC(O)R^{A}.

42. The compound of claim 40, wherein R⁷ and R⁹ each independently is selected from among hydrogen and C₁-C₆ alkyl.

43. A compound of Formula II: 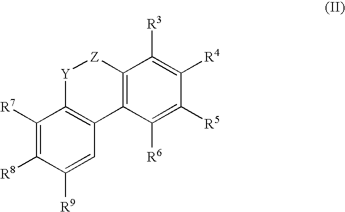
or a pharmaceutically acceptable salt or prodrug thereof, wherein:
R³ and R⁴ each independently is selected from among hydrogen, halogen, C₁-C₆ alkyl, C₁-C₆ haloalkyl, C₁-C₆ heteroalkyl and C₁-C₆ haloheteroalkyl;
R⁵ is selected from among hydrogen, halogen, OR^{A}, S(O)ₙR^{A}, NR^{A}R^{B}, NR^{A}S(O)ₙR^{B}, COR^{A}, CO₂R^{A}, OC(O)R^{A}, CH₂OR^{A}, CONR^{A}R^{B}, an optionally substituted C₁-C₆ alkyl, an optionally substituted C₁-C₆ haloalkyl and an optionally substituted C₁-C₆ heteroalkyl;
R⁶ is selected from among halogen, OR^{A}, S(O)ₙR^{A}, NR^{A}R^{B}, NR^{A}S(O)ₙR^{B}, COR^{A}, CO₂R^{A}, OC(O)R^{A}, CH₂OR^{A}, CONR^{A}R^{B}, an optionally substituted C₁-C₆ alkyl, an optionally substituted C₁-C₆ haloalkyl and an optionally substituted C₁-C₆ heteroalkyl; or
R⁵ and R⁶ are linked to form a heterocyclic ring;
R⁷ and R⁹ each independently is selected from among hydrogen, halogen, OR^{A}, C₁-C₆ alkyl, C₁-C₆ haloalkyl, C₁-C₆ heteroalkyl and C₁-C₆ haloheteroalkyl;
R⁸ is selected from among NO₂, CN, COR^{A}, CO₂R^{A}, CONR^{A}R^{B}, SOR^{A}, SO₂R^{A}, SO₂NR^{A}R^{B}, C₁-C₆ haloalkyl and C₁-C₄ haloheteroalkyl;
R^{A} and R^{B} each independently is selected from among hydrogen, C₁-C₆ alkyl, C₁-C₆ haloalkyl and C₁-C₆ heteroalkyl; or R^{A} and R^{B} are linked to form a ring;
Y and Z each independently is selected from among CR^{A}R^{B}, CR^{A}OR^{B}, CO, OCH₂, CH₂O, O, S(O)ₙ and NR^{A}; and
n is selected from among 0, 1 and 2;
provided that, if R⁸ is NO₂ and Y is CO and Z is O and R⁷ is hydrogen, then R⁹ is not hydrogen.

44. The compound of claim 43, wherein:
R³ and R⁴ each independently is selected from among hydrogen, halogen and C₁-C₆ alkyl; and
R^{A} and R^{B} each independently is selected from among hydrogen and C₁-C₆ alkyl.

45. The compound of claim 43, wherein:
R⁸ is selected from among NO₂, CN, COR^{A}, CO₂R^{A}, CONR^{A}R^{B}, SOR^{A}, SO₂R^{A} and SO₂NR^{A}R^{B}; and
Y and Z each independently is selected from among CR^{A}R^{B}, CR^{A}OR^{B}, CO, OCH₂, CH₂O and O.

46. The compound of claim 43, wherein:
R⁵ is selected from among hydrogen, halogen, OR^{A}, COR^{A}, CO₂R^{A}, OC(O)R^{A}, CH₂OR^{A}, CONR^{A}R^{B} and an optionally substituted C₁-C₆ alkyl; and
R⁶ is selected from among halogen, OR^{A}, COR^{A}, CO₂R^{A}, OC(O)R^{A}, CH₂OR^{A}, CONR^{A}R^{B} and an optionally substituted C₁-C₆ alkyl.

47. The compound of claim 43, wherein:
R⁷ and R⁹ each independently is selected from among hydrogen and C₁-C₆ alkyl; and
R⁸ is NO₂.

48. The compound of claim 43, wherein R³ and R⁴ are hydrogen.

49. The compound of claim 43, wherein:
R⁶ is selected from among OR^{A} and OC(O)R^{A}; and
Y and Z each independently is selected from among CO and O.

50. The compound of claim 43, wherein R⁵ is hydrogen.

51. A compound of Formula II: 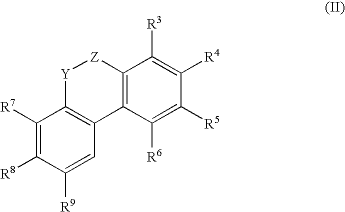
wherein:
R³ and R⁴ each independently is selected from among hydrogen, halogen, C₁-C₆ alkyl, C₁-C₆ haloalkyl, C₁-C₆ heteroalkyl and C₁-C₆ haloheteroalkyl;
R⁵ and R⁶ each independently is selected from among hydrogen, halogen, OR^{A}, S(O)ₙR^{A}, NR^{A}R^{B}, NR^{A}S(O)ₙR^{B}, COR^{A}, CO₂R^{A}, OC(O)R^{A}, CH₂OR^{A}, CONR^{A}R^{B}, an optionally substituted C₁-C₆ alkyl, an optionally substituted C₁-C₆ haloalkyl and an optionally substituted C₁-C₆ heteroalkyl; or
R⁵ and R⁶ are linked to form a heterocyclic ring;
R⁷ is selected from among hydrogen, halogen, OR^{A}, C₁-C₆ alkyl, C₁-C₆ haloalkyl, C₁-C₆ heteroalkyl and C₁-C₆ haloheteroalkyl;
R⁹ is selected from among hydrogen, halogen, OR^{A}, C₁-C₆ alkyl, C₁-C₆ haloalkyl, C₁-C₆ heteroalkyl and C₁-C₆ haloheteroalkyl;
R⁸ is selected from among NO₂, CN, COR^{A}, CO₂R^{A}, CONR^{A}R^{B}, SOR^{A}, SO₂R^{A}, SO₂NR^{A}R^{B}, C₁-C₆ haloalkyl and C₁-C₄ haloheteroalkyl;
R^{A} and R^{B} each independently is selected from among hydrogen, C₁-C₆ alkyl, C₁-C₆ haloalkyl and C₁-C₆ heteroalkyl; or
R^{A} and R^{B} are linked to form a ring;
Y and Z each independently is selected from among CR^{A}R^{B}, CR^{A}OR^{B}, CO, OCH₂, CH₂O, O, S(O)ₙ and NR^{A}; and
n is selected from among 0, 1 and 2;
provided that, if R⁸ is NO₂, CN, COR^{A}, CO₂R^{A} or CONR^{A}R^{B}, and Y or Z is CO, and R⁹ is hydrogen, then R⁷ is not hydrogen; and
provided that, if R⁸ is CN, CO₂R^{A} or COR^{A}, and R⁷ is methyl, then one of R⁵, R⁶ or R⁹ is not hydrogen; and
provided that Y and Z are not the same.

52. The compound of claim 51, wherein R⁵ and R⁶ each independently is selected from among hydrogen, halogen, OR^{A}, COR^{A}, CO₂R^{A}, OC(O)R^{A}, CH₂OR^{A}, CONR^{A}R^{B}, an optionally substituted C₁-C₆ alkyl, an optionally substituted C₁-C₆ haloalkyl and an optionally substituted C₁-C₆ heteroalkyl.

53. The compound of claim 51, wherein:
R⁷ is selected from among halogen, OR^{A}, C₁-C₆ alkyl, C₁-C₆ haloalkyl, C₁-C₆ heteroalkyl and C₁-C₆ haloheteroalkyl; and
R⁸ is selected from among NO₂, CN, COR^{A}, CO₂R^{A}, CONR^{A}R^{B}, SOR^{A}, SO₂R^{A} and SO₂NR^{A}R^{B}.

54. The compound of claim 51, wherein R⁸ is selected from among NO₂, SOR^{A}, SO₂R^{A} and SO₂NR^{A}R^{B}.

55. The compound of claim 51, wherein:
R⁷ is selected from among halogen, OR^{A}, C₁-C₆ alkyl, C₁-C₆ haloalkyl, C₁-C₆ heteroalkyl and C₁-C₆ haloheteroalkyl; and
R⁸ is NO₂.

56. The compound of claim 51, wherein:
R³ and R⁴ each independently is selected from among hydrogen, halogen, C₁-C₆ alkyl, C₁-C₆ haloalkyl; and
R⁷ is selected from among halogen, OR^{A}, C₁-C₆ alkyl and C₁-C₆ haloalkyl.

57. The compound of claim 51, wherein:
R⁵ and R⁶ each independently is selected from among hydrogen, halogen, OR^{A}, COR^{A}, CO₂R^{A}, OC(O)R^{A}, CH₂OR^{A}, CONR^{A}R^{B}, an optionally substituted C₁-C₆ alkyl and an optionally substituted C₁-C₆ haloalkyl;
R⁹ is selected from among hydrogen, halogen, OR^{A}, C₁-C₆ alkyl and C₁-C₆ haloalkyl; and
Y and Z each independently is selected from among CR^{A}R^{B}, CR^{A}OR^{B}, CO, OCH₂, CH₂O and O.

58. The compound of claim 51, wherein:
R^{A} and R^{B} are each independently selected from among hydrogen, C₁-C₆ alkyl and C₁-C₆ haloalkyl; and
Y and Z each independently is selected from among CR^{A}R^{B}, CR^{A}OR^{B}, CO and O.

59. The compound of claim 51, wherein:
R³ and R⁴ are hydrogen;
R⁷ is selected from among C₁-C₆ alkyl and C₁-C₆ haloalkyl;
R⁹ is selected from among hydrogen, halogen, OR^{A}, C₁-C₆ alkyl and C₁-C₆ haloalkyl;
R^{A} and R^{B} each independently is selected from among hydrogen, C₁-C₆ alkyl and C₁-C₆ haloalkyl; and
Y and Z each independently is selected from among CR^{A}R^{B}, CR^{A}OR^{B}, CO and O.

60. The compound of claim 51, wherein:
R³ and R⁴ are hydrogen;
R⁵ and R⁶ each independently is selected from among hydrogen, OR^{A}, COR^{A}, CO₂R^{A}, OC(O)R^{A}, CH₂OR^{A}, CONR^{A}R^{B} and an optionally substituted C₁-C₆ alkyl;
R⁷ is selected from among C₁-C₆ alkyl and C₁-C₆ haloalkyl;
R⁹ is selected from among hydrogen, halogen, OR^{A}, C₁-C₆ alkyl and C₁-C₆ haloalkyl;
R⁸ is NO₂;
R^{A} and R^{B} each independently is selected from among hydrogen, C₁-C₆ alkyl and C₁-C₆ haloalkyl; and
Y and Z are each independently selected from among CO and O.

61. The compound of claim 51, wherein:
R³ and R⁴ are hydrogen;
R⁵ and R⁶ each independently is selected from among hydrogen, OR^{A} and OC(O)R^{A};
R⁷ is selected from among C₁-C₆ alkyl and C₁-C₆ haloalkyl;
R⁹ is hydrogen;
R⁸ is NO₂;
R^{A} and R^{B} each independently is selected from among hydrogen, C₁-C₆ alkyl and C₁-C₆ haloalkyl; and
Y and Z each independently is selected from among CO and O.

62. The compound of claim 1 of Formula II, or a pharmaceutically acceptable salt or prodrug thereof, wherein:
R⁷ is not hydrogen;
R⁸ is NO₂;
R⁹ is hydrogen;
Y is CO; and
Z is O.

63. The compound of claim 1 of Formula II, or a pharmaceutically acceptable salt or prodrug thereof, wherein:
R⁷ is not hydrogen;
R⁸ is selected from among NO₂, CN, COR^{A}, CO₂R^{A} and CONR^{A}R^{B};
R⁹ is hydrogen; and
Y or Z is CO.

64. The compound of claim 1 having a structure of Formula II, or a pharmaceutically acceptable salt or prodrug thereof, wherein:
R⁷ is not hydrogen;
R⁸ is COR^{A} or CO₂R^{A}; and
R⁹ is methoxy.

65. The compound of claim 1 of Formula II, or a pharmaceutically acceptable salt or prodrug thereof, wherein:
R⁷ is methyl;
R⁸ is CN or CO₂R^{A}; and
one of R⁵, R⁶ or R⁹ is not hydrogen.

66. A compound selected from among:
9-Fluoro-4-methyl-1H-6-oxa-1-aza-chrysene-2,5-dione (Compound 101);
2,2-Dimethyl-propionic acid 10-methoxy-4-methyl-2,5-dioxo-2,5-dihydro-1H-6-oxa-1-aza-chrysen-9-yl ester (Compound 102);
9-Methoxy-4-methyl-1H-6-oxa-1-aza-chrysene-2,5-dione (Compound 103);
2,2-Dimethyl-propionic acid 4-methyl-2,5-dioxo-2,5-dihydro-1H-6-oxa-1-aza-chrysen-10-yl ester (Compound 104);
5-Hydroxy-10-methoxy-4-methyl-1H,5H-6-oxa-1-aza-chrysen-2-one (Compound 105);
5,10-Dimethoxy-4-methyl-1H,5H-6-oxa-1-aza-chrysen-2-one (Compound 106);
(±)-10-Methoxy-4,5-dimethyl-1H,5H-6-oxa-1-aza-chrysen-2-one (Compound 107);
(±)-5,10-Dimethoxy-4,5-dimethyl-1H,5H-6-oxa-1-aza-chrysen-2-one (Compound 108);
10-Methoxy-4-methyl-1H,5H-6-oxa-1-aza-chrysen-2-one (Compound 109);
5-Allyl-10-methoxy-4-methyl-1H,5H-6-oxa-1-aza-chrysen-2-one (Compound 110);
4-Methyl-2,5-dioxo-2,5-dihydro-1H-6-oxa-1-aza-chrysene-10-carboxylic acid methyl ester (Compound 111);
10-Hydroxymethyl-4-methyl-1H,5H-6-oxa-1-aza-chrysen-2-one (Compound 112);
10-Hydroxymethyl-4-trifluoromethyl-1H,5H-6-oxa-1-aza-chrysen-2-one (Compound 113);
5-Hydroxy-10-methoxy-4-trifluoromethyl-1H,5H-6-oxa-1-aza-chrysen-2-one (Compound 114);
10-Hydroxy-4-trifluoromethyl-1H,5H-6-oxa-1-aza-chrysen-2-one (Compound 115);
2,2-Dimethyl-propionic acid 2-oxo-4-trifluoromethyl-2,5-dihydro-1H-6-oxa-1-aza-chrysen-10-yl ester (Compound 116);
9-Hydroxy-10-methoxy-4-methyl-1H-6-oxa-1-aza-chrysene-2,5-dione (Compound 117);
9-Hydroxy-5,10-dimethoxy-4-methyl-1H,5H-6-oxa-1-aza-chrysen-2-one (Compound 118);
9-Hydroxy-10-methoxy-4-methyl-1H,5H-6-oxa-1-aza-chrysen-2-one (Compound 119);
9-Isopropoxy-10-methoxy-4-methyl-1H-6-oxa-1-aza-chrysene-2,5-dione (Compound 120);
9-Ethoxy-10-methoxy-4-methyl-1H-6-oxa-1-aza-chrysene-2,5-dione (Compound 121);
9-Ethoxy-1-ethyl-10-methoxy-4-methyl-1H-6-oxa-1-aza-chrysene-2,5-dione (Compound 122);
4-Methoxy-10-methyl-7,13-dihydro-12-oxa-7-aza-benzo[3,4]cyclohepta[1,2-α]-naphthalene-8,11-dione (Compound 123);
10-Hydroxymethyl-4-methyl-1H-6-oxa-1-aza-chrysene-2,5-dione (Compound 124);
10-Hydroxy-4-trifluoromethyl-1H-5-oxa-1-aza-chrysene-2,6-dione (Compound 125);
10-Methoxy-4-trifluoromethyl-1H-5-oxa-1-aza-chrysene-2,6-dione (Compound 126);
10-Methoxy-4-trifluoromethyl-1,6-dihydro-5-oxa-1-aza-chrysen-2-one (Compound 127);
4-Trifluoromethyl-1,6-dihydro-5-oxa-1-aza-chrysen-2-one (Compound 128);
1-Hydroxy-7-methyl-8-nitro-benzo[c]chromen-6-one (Compound 129); and
1-Methoxy-7-methyl-8-nitro-benzo[c]chromen-6-one (Compound 130);
and pharmaceutically acceptable salts and prodrugs thereof.

67. The compound of claim 1, wherein the compound is a selective androgen receptor modulator.

68. The compound of claim 67, wherein the selective androgen receptor modulator is a selective androgen receptor agonist.

69. The compound of claim 67, wherein the selective androgen receptor modulator is a selective androgen receptor antagonist.

70. The compound of claim 67, wherein the selective androgen receptor modulator is a selective androgen receptor partial agonist.

71. The compound of claim 67, wherein the compound is a tissue-specific modulator.

72. The compound of claim 1, wherein the compound is a selective androgen receptor binding compound.

73. The compound of claim 1, wherein the compound is a selective androgen receptor reducing compound.

74. The compound of claim 73, wherein the compound is a selective androgen receptor degrading compound.

75. A method for modulating an activity of an androgen receptor, comprising contacting an androgen receptor with a compound of Formula I or Formula II: 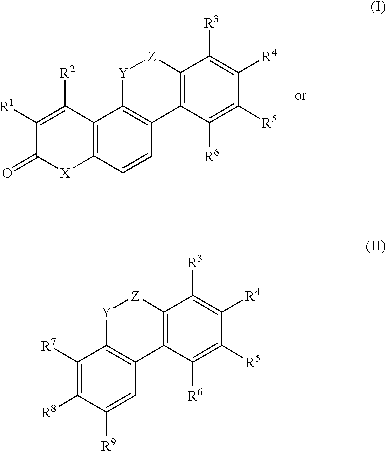
wherein:
R¹ is selected from among hydrogen, F, Cl, C₁-C₄ alkyl and C₁-C₄ haloalkyl;
R² is selected from among hydrogen, halogen, C₁-C₆ alkyl, C₁-C₆ haloalkyl, C₁-C₆ heteroalkyl and C₁-C₄ haloheteroalkyl;
R³ and R⁴ each independently is selected from among hydrogen, halogen, C₁-C₆ alkyl, C₁-C₆ haloalkyl, C₁-C₆ heteroalkyl and C₁-C₆ haloheteroalkyl;
R⁵ and R⁶ each independently is selected from among hydrogen, halogen, OR^{A}, S(O)ₙR^{A}, NR^{A}R^{B}, NR^{A}S(O)ₙR^{B}, COR^{A}, CO₂R^{A}, OC(O)R^{A}, CH₂OR^{A}, CONR^{A}R^{B}, an optionally substituted C₁-C₆ alkyl, an optionally substituted C₁-C₆ haloalkyl and an optionally substituted C₁-C₆ heteroalkyl; or R⁵ and R⁶ are linked to form a heterocyclic ring;
R⁷ and R⁹ each independently is selected from among hydrogen, halogen, OR^{A}, C₁-C₆ alkyl, C₁-C₆ haloalkyl, C₁-C₆ heteroalkyl and C₁-C₆ haloheteroalkyl;
R⁸ is selected from among NO₂, CN, COR^{A}, CO₂R^{A}, CONR^{A}R^{B}, SOR^{A}, SO₂R^{A}, SO₂NR^{A}R^{B}, C₁-C₆ haloalkyl and C₁-C₄ haloheteroalkyl;
R^{A} and R^{B} each independently is selected from among hydrogen, C₁-C₆ alkyl, C₁-C₆ haloalkyl and C₁-C₆ heteroalkyl; or R^{A} and R^{B} are linked to form a ring;
X is selected from among O, S and NR^{A};
Y and Z each independently is selected from among CR^{A}R^{B}, CR^{A}OR^{B}, CO, OCH₂, CH₂O, O, S(O)ₙ and NR^{A}; and
n is selected from among 0, 1 and 2; and
pharmaceutically acceptable salts and prodrugs thereof.

76. The method of claim 75, wherein if X is NH, and Y is CO, and Z is O, and each of R¹, R³, R⁴, and R⁵ is hydrogen, and R² is CH₃, then R⁶ is not OCH₃.

77. The method of claim 75, wherein:
if R⁸ is NO₂, CN, COR^{A}, CO₂R^{A} and CONR^{A}R^{B}, and R⁹ is hydrogen, then R⁷ is not hydrogen; and
if R⁸ is COR^{A} or CO₂R^{A}, and R⁹ is methoxy, then R⁷ is not hydrogen; and
if R⁸ is CN or COR^{A}, and R⁷ is methyl, then R⁹ is not hydrogen; and
if the compound has a structure of Formula II, then Y and Z are not the same.

78. The method of claim 75, wherein the androgen receptor is in a cell.

79. A method for decreasing the amount of androgen receptor in cells, comprising contacting the cells with a compound of Formula I or Formula II: 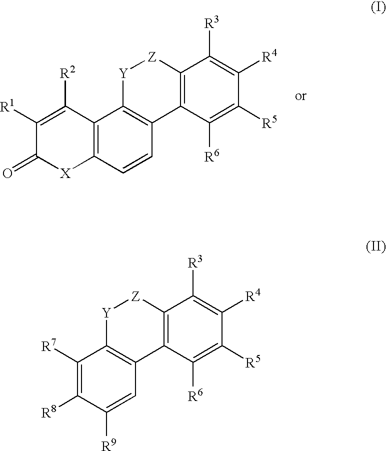
wherein:
R¹ is selected from among hydrogen, F, Cl, C₁-C₄ alkyl and C₁-C₄ haloalkyl;
R² is selected from among hydrogen, halogen, C₁-C₆ alkyl, C₁-C₆ haloalkyl, C₁-C₆ heteroalkyl and C₁-C₄ haloheteroalkyl;
R³ and R⁴ each independently is selected from among hydrogen, halogen, C₁-C₆ alkyl, C₁-C₆ haloalkyl, C₁-C₆ heteroalkyl and C₁-C₆ haloheteroalkyl;
R⁵ and R⁶ each independently is selected from among hydrogen, halogen, OR^{A}, S(O)ₙR^{A}, NR^{A}R^{B}, NR^{A}S(O)ₙR^{B}, COR^{A}, CO₂R^{A}, OC(O)R^{A}, CH₂OR^{A}, CONR^{A}R^{B}, an optionally substituted C₁-C₆ alkyl, an optionally substituted C₁-C₆ haloalkyl and an optionally substituted C₁-C₆ heteroalkyl; or R⁵ and R⁶ are linked to form a heterocyclic ring;
R⁷ and R⁹ each independently is selected from among hydrogen, halogen, OR^{A}, C₁-C₆ alkyl, C₁-C₆ haloalkyl, C₁-C₆ heteroalkyl and C₁-C₆ haloheteroalkyl;
R⁸ is selected from among NO₂, CN, COR^{A}, CO₂R^{A}, CONR^{A}R^{B}, SOR^{A}, SO₂R^{A}, SO₂NR^{A}R^{B}, C₁-C₆ haloalkyl and C₁-C₄ haloheteroalkyl;
R^{A} and R^{B} each independently is selected from among hydrogen, C₁-C₆ alkyl, C₁-C₆ haloalkyl and C₁-C₆ heteroalkyl; or R^{A} and R^{B} are linked to form a ring;
X is selected from among O, S and NR^{A};
Y and Z each independently is selected from among CR^{A}R^{B}, CR^{A}OR^{B}, CO, OCH₂, CH₂O, O, S(O)ₙ and NR^{A}; and
n is selected from among 0, 1 and 2; and
pharmaceutically acceptable salts and prodrugs thereof,
wherein the amount of androgen receptors in the cells is decreased.

80. The method of claim 79, wherein if X is NH, and Y is CO, and Z is O, and each of R¹, R³, R⁴, and R⁵ is hydrogen, and R² is CH₃, then R⁶ is not OCH₃.

81. The method of claim 79, wherein:
if R⁸ is NO₂, CN, COR^{A}, CO₂R^{A} and CONR^{A}R^{B}, and R⁹ is hydrogen, then R⁷ is not hydrogen; and
if R⁸ is COR^{A} or CO₂R^{A}, and R⁹ is methoxy, then R⁷ is not hydrogen; and
if R⁸ is CN or COR^{A}, and R⁷ is methyl, then R⁹ is not hydrogen; and
if the compound has a structure of Formula II, then Y and Z are not the same.

82. A method for treating a patient having a condition susceptible to treatment with an androgen receptor modulator, comprising:
administering to the patient a pharmaceutical agent comprising a compound of Formula I or Formula II: 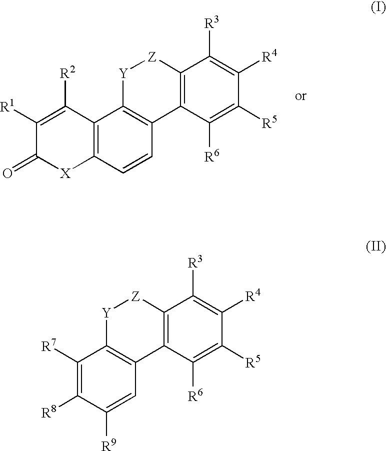
wherein:
R¹ is selected from among hydrogen, F, Cl, C₁-C₄ alkyl and C₁-C₄ haloalkyl;
R² is selected from among hydrogen, halogen, C₁-C₆ alkyl, C₁-C₆ haloalkyl, C₁-C₆ heteroalkyl and C₁-C₄ haloheteroalkyl;
R³ and R⁴ each independently is selected from among hydrogen, halogen, C₁-C₆ alkyl, C₁-C₆ haloalkyl, C₁-C₆ heteroalkyl and C₁-C₆ haloheteroalkyl;
R⁵ and R⁶ each independently is selected from among hydrogen, halogen, OR^{A}, S(O)ₙR^{A}, NR^{A}R^{B}, NR^{A}S(O)ₙR^{B}, COR^{A}, CO₂R^{A}, OC(O)R^{A}, CH₂OR^{A}, CONR^{A}R^{B}, an optionally substituted C₁-C₆ alkyl, an optionally substituted C₁-C₆ haloalkyl and an optionally substituted C₁-C₆ heteroalkyl; or R⁵ and R⁶ are linked to form a heterocyclic ring;
R⁷ and R⁹ each independently is selected from among hydrogen, halogen, OR^{A}, C₁-C₆ alkyl, C₁-C₆ haloalkyl, C₁-C₆ heteroalkyl and C₁-C₆ haloheteroalkyl;
R⁸ is selected from among NO₂, CN, COR^{A}, CO₂R^{A}, CONR^{A}R^{B}, SOR^{A}, SO₂R^{A}, SO₂NR^{A}R^{B}, C₁-C₆ haloalkyl and C₁-C₄ haloheteroalkyl;
R^{A} and R^{B} each independently is selected from among hydrogen, C₁-C₆ alkyl, C₁-C₆ haloalkyl and C₁-C₆ heteroalkyl; or R^{A} and R^{B} are linked to form a ring;
X is selected from among O, S and NR^{A};
Y and Z each independently is selected from among CR^{A}R^{B}, CR^{A}OR^{B}, CO, OCH₂, CH₂O, O, S(O)ₙ and NR^{A}; and
n is selected from among 0, 1 and 2; and
pharmaceutically acceptable salts and prodrugs thereof,
whereby one or more symptoms of the condition is ameliorated.

83. The method of claim 82, further comprising identifying a patient having a condition susceptible to treatment with an androgen receptor modulator.

84. The method of claim 82, wherein the condition is selected from among frailty or age-related functional decline in the elderly; catabolic side effects of glucocorticoids; osteoporosis; osteopenia; chronic fatigue syndrome; chronic myalgia; acute fatigue syndrome and muscle loss; wound healing; bone fracture repair; post-surgical adhesions; periodontal disease; wasting secondary to fractures; wasting in connection with chronic obstructive pulmonary disease; wasting in connection with chronic liver disease; wasting in connection with AIDS, cancer cachexia, burn and trauma recovery, chronic catabolic state, eating disorders and chemotherapy; cardiomyopathy; thrombocytopenia; growth retardation in connection with Crohn's disease; short bowel syndrome; irritable bowel syndrome; inflammatory bowel disease; Crohn's disease; ulcerative colitis; complications associated with transplantation; physiological short stature associated with growth hormone deficiency; short stature associated with chronic illness; obesity; growth retardation associated with obesity; anorexia; hypercortisolism; Cushing's syndrome; Paget's disease; Alzheimer's disease; osteoarthritis; pulsatile growth hormone release; osteochondrodysplasias; depression, nervousness, irritability or stress; reduced mental energy; low self-esteem; catabolism in connection with pulmonary dysfunction and ventilator dependency; cardiac dysfunction; elevated blood pressure; ventricular dysfunction; reperfusion events; chronic dialysis; protein catabolic responses following trauma; cachexia and protein loss due to chronic illness; hyperinsulinemia; nesidioblastosis; wasting in connection with multiple sclerosis or other neurodegenerative disorders; metabolic homeostasis and renal homeostasis; insulin resistance; insulin resistance in the heart; hypothermia; congestive heart failure; lipodystrophy; muscular atrophy; musculoskeletal impairment; sleep disorders; catabolic state of prolonged critical illness; hirsutism; acne; seborrhea; androgenic alopecia; anemia; hyperpilosity; benign prostate hypertrophy; adenomas and neoplasms of the prostate; malignant tumor cells containing the androgen receptor; cancers of the skin, pancreas, endometrium, lung, colon, breast, brain, ovaries, bladder, lympathic, liver and kidney; osteosarcoma; hypercalcemia of malignancy; metastatic bone disease; spermatogenesis; endometriosis; polycystic ovary syndrome; preeclampsia; eclampsia of pregnancy; preterm labor; premenstrual syndrome; vaginal dryness; age related decreased testosterone levels in men; male menopause; hypogonadism; male and female sexual dysfunction; hair loss; and Reaven's Syndrome.

85. The method of claim 82, wherein the patient has a condition selected from among of acne, male-pattern baldness, wasting diseases, hirsutism, hypogonadism, osteoporoses, infertility, impotence and cancer.

86. The method of claim 85, wherein the patient has prostate cancer.

87. The method of claim 86, wherein the patient has androgen dependent prostate cancer.

88. The method of claim 86, wherein the patient has androgen independent prostate cancer.

89. A method for prevention, treatment or amelioration of one or more symptoms of a disease or a disorder associated with androgen receptor activity, comprising:
administering to the patient a pharmaceutical agent comprising a compound of any of claims 1-66, wherein the symptom(s) of the disease or disorder are selected from among loss of muscle strength or function; frailty or age-related functional decline (“AFRD”); reduced bone mass, density or growth; bone fracture; loss of sensory function; wasting; anorexia; growth retardation; complications associated with transplantation; depression; nervousness; irritability; stress; reduced mental energy; loss of cognitive function; dementia; short term memory loss; high blood pressure; catabolic aging; myelin dysfunction; loss of cartilage; loss of skin thickness; increase in rapid eye movement (REM) sleep; decrease in REM latency; muscular atrophy; musculoskeletal impairment; anemia; vaginal dryness; and decreased testosterone levels in men.

90. A method of male or female contraception, comprising:
administering to a subject a pharmaceutical agent comprising a compound of claim 1, whereby conception or impregnation is prevented.

91. A pharmaceutical composition, comprising a compound of claim 1 and a pharmaceutically acceptable carrier.

92-95. (canceled)

96. An article of manufacture, comprising:
a packaging material;
a compound of claim 1 or pharmaceutically acceptable derivative thereof which is effective for modulating the activity of androgen receptor, or for treatment, prevention or amelioration of one or more symptoms of androgen receptor mediated diseases or disorders, or diseases or disorders in which androgen receptor activity is implicated, within the packaging material; and
a label that indicates that the compound is used for modulating the activity of androgen receptor or for treatment, prevention or amelioration of one or more symptoms of androgen receptor mediated diseases or disorders, or diseases or disorders in which androgen receptor activity is implicated.
